(19) United States
(12) Patent Application Publication
(10) Pub. No.: US 2024/0181022 A1
(43) Pub. Date: Jun. 6, 2024

(54) AMURINS LYSINS, AND LYSIN-ANTIMICROBIAL PEPTIDE (AMP) CONSTRUCTS ACTIVE AGAINST GRAM-NEGATIVE BACTERIA

(71) Applicant: ContraFect Corporation, Yonkers, NY (US)

(72) Inventor: Raymond SCHUCH, Mountain Lakes, NJ (US)

(21) Appl. No.: 18/552,316

(22) PCT Filed: Mar. 23, 2022
(86) PCT No.: PCT/US2022/021543

§ 371 Date: Sep. 25, 2023

Related U.S. Application Data
(60) Provisional application No. 63/166,463, filed on Mar. 26, 2021.
(60) Provisional application No. 63/196,436, filed on Jun. 3, 2021.
(60) Provisional application No. 63/249,638, filed on Sep. 29, 2021.
(60) Provisional application No. 63/279,855, filed on Nov. 16, 2021.
(60) Provisional application No. 63/282,230, filed on Nov. 23, 2021.

(51) Int. Cl. A61K 38/47; A61K 38/16; A61K 45/06; A61K 47/64; A61P 31/04

(52) U.S. Cl. CPC A61K 38/47; A61K 38/162; A61K 45/06; A61K 47/64; A61P 31/04

(57) ABSTRACT

Disclosed herein are methods of inhibiting the growth, reducing the population, or killing of at least one species of Gram-negative bacteria comprising contacting the bacteria with a composition comprising an effective amount of a Chp peptide, lysin, or lysin-AMP construct or active fragments or variants thereof for a period of time sufficient to inhibit said growth, reduce said population, or kill said at least one species of Gram-negative bacteria. Also disclosed herein are methods of inhibiting a Gram-negative bacteria present in sputum; methods of preventing, disrupting or eradicating a Gram-negative bacterial biofilm; and methods of treating a bacterial infection caused by a Gram-negative bacteria.

### CROSS-REFERENCE TO RELATED APPLICATIONS

This application claims the benefit of, and relies on the filing date of, U.S. Provisional Application No. 63/282,230, filed 23 Nov. 2021; U.S. Provisional Application No. 63/279,855, filed 16 Nov. 2021; U.S. Provisional Application No. 63/249,638, which was filed on 29 Sep. 2021; U.S. Provisional Application No. 63/196,436, filed 3 Jun. 2021; and U.S. Provisional Application No. 63/166,463, which was filed on 26 Mar. 2021, each of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 23, 2022, is named 0341_0029-PCT_SL.txt and is 126,409 bytes in size.

### FIELD OF THE DISCLOSURE

The present disclosure relates to the field of antimicrobial agents and more specifically to phage-derived antimicrobial amurin peptides and lysin polypeptides that infect Gram-negative and/or acid-fast bacteria and the use of these peptides in killing Gram-negative and/or acid-fast (particularly MDR and/or XDR) bacteria and combatting bacterial infection and contamination, particularly those present in the pulmonary system, particularly in the sputum.

### BACKGROUND OF THE DISCLOSURE

Gram-negative bacteria, in particular, members of the genus Pseudomonas and the emerging multi-drug resistant pathogen Acinetobacter baumannii, are an important cause of serious and potentially life-threatening invasive infections. Pseudomonas infection presents a major problem in burn wounds, chronic wounds, chronic obstructive pulmonary disorder (COPD), cystic fibrosis, surface growth on implanted biomaterials, and within hospital surface and water supplies where it poses a host of threats to vulnerable patients.

Once established in a patient, P. aeruginosa can be especially difficult to treat. The genome encodes a host of resistance genes, including multidrug efflux pumps and enzymes conferring resistance to beta-lactam and aminoglycoside antibiotics, making therapy against this Gram-negative pathogen particularly challenging due to the lack of novel antimicrobial therapeutics. This challenge is compounded by the ability of P. aeruginosa to grow in a biofilm, which may enhance its ability to cause infections by protecting bacteria from host defenses and chemotherapy.

In the healthcare setting, the incidence of drug-resistant strains of P. aeruginosa is increasing. In an observational study of health care-associated bloodstream infections (BSIs) in community hospitals, P. aeruginosa was one of the top four Multiple Drug Resistant (MDR) pathogens, contributing to an overall hospital mortality of 18%. Additionally, outbreaks of MDR P. aeruginosa are well-documented. Poor outcomes are associated with MDR strains of P. aeruginosa that frequently require treatment with drugs of last resort, such as colistin. Additionally, the emergence of Extensively Drug Resistant (XDR) bacteria, including XDR strains of P. aeruginosa, is increasing, such that there may be fewer, if any, effective antibiotic treatment agents available.

Other drug-resistant bacteria that have been identified as significant threats by the World Health Organization (WHO) and Centers for Disease Control (CDC) include the following Gram-negative bacteria: Acinetobacter baumannii, Pseudomonas aeruginosa, Enterobacteriaceae (including Escherichia coli, Klebsiella pneumoniae, and Enterobacter cloacae), Salmonella species, Neisseria gonorrhoeae, and Shigella species (Tillotson G. 2018. A crucial list of pathogens. Lancet Infect Dis 18:234-236).

Acid-fast bacteria, which generally have a high content of mycolic acid in their cell walls, can be identified by measuring the bacteria's resistance to decolorization by acids during laboratory staining such as Ziehl-Neelsen staining. Acid-fast bacteria can resist the decolorization of acid-based stains. Like Gram-negative bacteria, acid-fast bacteria, e.g., actinobacteria, are responsible for life-threatening diseases. For example, mycobacterium is a genus of actinobacteria and includes pathogens known to cause serious diseases including tuberculosis and leprosy. Mycobacterium tuberculosis usually presents by infecting the lungs and may spread through the air when an infected subject coughs, sneezes, or speaks, for example. Like P. aeruginosa, M. tuberculosis also has an increasing incidence of drug-resistant strains, making tuberculosis infections increasingly more difficult to treat.

Moreover, reduced effectiveness of certain antibiotics is observed in combating both Gram-negative and Gram-positive infections due to factors in the environment of the infection, such as the pulmonary surfactant, rather than to antibiotic resistance developments. Certain antibiotics, such as daptomycin, for example, have failed to meet criteria in a clinical trial for severe community-acquired pneumonia. This deficiency has been shown to be due to an interaction between daptomycin and pulmonary surfactant, which inhibits the activity of this antibiotic against Gram-positive organisms, specifically in the lung environment and more generally in the airway environment wherein pulmonary surfactant is present. Silverman, J. A. et al., “Surfactant Inhibition of Daptomycin,” JID, 191: 2149-2152 (2005). Thus, daptomycin is not indicated for treatment of lung and more generally airway (especially lower respiratory tract) infections and those of skill in the art would not employ a treatment regimen including daptomycin to treat such infections. The inability of daptomycin to combat infection in the presence of pulmonary surfactants has been shown dramatically in, for example, Koplowicz, Y. B. et al., “Development of daptomycin-susceptible methicillin-resistant Staphylococcus aureus Pneumonia during high-dose daptomycin therapy”, Clin Infect Dis. 49(8):1286-7 (2009). Recent studies have focused on overcoming daptomycin inactivity in the presence of surfactant by testing and evaluating antibacterial activity of hybrid molecules of the structurally related lipopeptide A54145. Nguyen, K. T. et al., “Genetically engineered lipopeptide antibiotics related to A54145 and daptomycin with improved properties”, Antimicrob. Agents Chemother. 2010 April; 54(4):1404-1413.

Pulmonary surfactant, a primary component of epithelial lining fluid, is a complex lipid-and-protein mixture that coats the interior surface of the airway, reducing surface tension within the alveoli. Surfactant is composed primarily of dipalmitoylphosphatidylcholine (˜80% in all mammalian species), along with significant amounts of phosphatidylglycerol (PG) and smaller amounts of minor phospholipids, neutral lipids, and cholesterol. There are 4 protein components: hydrophilic proteins SP-A and SP-D and hydrophobic proteins SP-B and SP-C. Goerke, J., “Pulmonary Surfactant: functions and molecular composition”, Biochim. Biophys. Acta. 1998; 1408:79-89. Daptomycin is inserted into artificial membrane vesicles composed of phosphatidylcholine (PC) and PC/PG. Lakey J. H. et al., “Fluorescence indicates a calcium-dependent interaction between the lipopeptide antibiotic LY146032 and phospholipid membranes,” Biochemistry 1988; 27:4639-45; Jung, D. et al., “Structural transitions as determinants of the action of the calcium-dependent antibiotic daptomycin”, Chem. Biol. 2004; 11:949-57.

Thus, to the extent that otherwise effective antibiotics are inhibited by factors present in the organ or tissue that is the site of the infection, such as pulmonary surfactant in the case of infections of the lungs or other airways and more generally of the respiratory tract, a treatment regimen that would restore and even augment activity of such antibiotics would be of commercial and public health value.

In addition to daptomycin discussed above, other antibiotics that are known to be inhibited by pulmonary surfactant include without limitation: tobramycin, an aminoglycoside used to treat infections caused by the gram-negative bacterium Pseudomonas aeruginosa, a common cause of pneumonia (van't Veen, A. et al., “Influence of pulmonary surfactant on in vitro bactericidal activities of amoxicillin, ceftazidime, and tobramycin”, Antimicrob. Agents Chemother. 39:329-333 (1995)), and colistin, a cyclic lipopeptide (polymyxin) broadly active against gram-negative bacteria, including P. aeruginosa. Schwameis, R. et al., “Effect of Pulmonary surfactant on antimicrobial activity in vitro”, Antimicrob. Agents Chemother. 57(10):5151-54 (2013).

Moreover, for patients with bacterial infections of the pulmonary system, such as cystic fibrosis patients with bacterial infections of the lungs, the patient's sputum has an altered composition in comparison to non-cystic fibrosis patients, wherein the sputum from cystic fibrosis patients has been shown to have a higher concentration of DNA, proteins, lipids, and carbohydrates. Barth, A. L., The high amino-acid content of sputum from cystic fibrosis patients promotes growth of auxotrophic Pseudomonas aeruginosa, J. Med. Microbiol. 1996, 45:110-119. This difference in sputum composition is thought to be a secondary result of bacterial infection in the lungs. Therefore, antibacterial agents having activity in sputum may be used to predict the agents' activity against bacterial infection in patients, such as patients with lung disease, e.g. cystic fibrosis.

To address the need for new antimicrobials with novel mechanisms, researchers are investigating a variety of drugs and biologics. One such class of antimicrobial agents includes lysins. Lysins are cell wall peptidoglycan hydrolases, which act as “molecular scissors” to degrade the peptidoglycan meshwork responsible for maintaining cell shape and for withstanding internal osmotic pressure. Degradation of peptidoglycan results in osmotic lysis. However, certain lysins have not been effective against Gram-negative bacteria, at least in part, due to the presence of an outer membrane (OM), which is absent in Gram-positive bacteria and which limits access to subjacent peptidoglycan. Modified lysins (“artilysins”) have also been developed. These agents, which contain lysins fused to specific α-helical domains with polycationic, amphipathic, and hydrophobic features, are capable of translocating across the OM. However, certain artilysins exhibit low in vivo activity. This may be caused by constituents of human serum and specifically by physiologic salt and divalent cations. These constituents compete for lipopolysaccharide binding sites and may interfere with the α-helical translocation domains of lysins, thereby restricting activity in blood and limiting the effectiveness of certain lysins and artilysins for treating invasive infections. A similar lack of activity in blood has been reported for multiple different outer membrane-penetrating and destabilizing antimicrobial peptides.

In addition to lysins and artilysins, other phage-encoded host lysis systems have been identified, including “amurins” (Chamakura K R et al., 2017. Mutational analysis of the MS2 lysis protein L. Microbiology 163:961-969). The term amurin describes a limited set of nonmuralytic (not “wall-destroying,” i.e., not based on peptidoglycan hydrolysis of the cell wall) lysis activities from both ssDNA and ssRNA phages (Microviridae and Leviviridae, respectively). For example, the protein E amurin of phage (pX174 (Family Microviridae, genus Microvirus) is a 91 amino acid membrane protein that causes lysis by inhibiting the bacterial translocase MraY, an essential membrane-embedded enzyme that catalyzes the formation of the murein precursor, Lipid I (Zheng Y et al., 2009. Purification and functional characterization of phiX174 lysis protein E. Biochemistry 48:4999-5006). Additionally, the A2 capsid protein of phage Qβ (Family Leviviridae, genus Allolevivirus) is a 420-amino acid structural protein (and amurin) that causes lysis by interfering with MurA activity and dysregulating the process of peptidoglycan biosynthesis (Gorzelnik K V et al., 2016. Proc Natl Acad Sci USA 113:11519-11524). Other non-limiting examples include the LysM amurin of phage M, which is a specific inhibitor of MurJ, the lipid II flippase of E. coli, and the protein L amurin of phage MS2 (Family Levivirdae, genus Levivirus), which is a 75 amino acid integral membrane protein and causes lysis in a manner requiring the activity of host chaperone DnaJ (Chamakura K R et al., 2017. J Bacteriol 199). A putative domain structure for the L-like amurins has been assigned and includes an internal leucylserine dipeptide immediately preceded by a stretch of 10-17 hydrophobic residues. These amurins are integral membrane proteins and have not been purified and used like lysins. Further, their targets are in the cytoplasm. They have not been tested as lytic agents. Some amurins have been described in detail, for example in PCT Published Application No. WO 2001/009382, but at best they constitute a basis for development of therapeutics and have not been developed into antibacterial therapeutics.

Although recent publications have described lysins/artilysins and other host lysis systems (e.g., amurins) that may be used against Gram-negative bacteria with varying levels of efficacy in vivo, there remains a need for additional antibacterial compounds that target MDR and/or XDR bacteria, including P. aeruginosa, S. maltophilia, A. xylosoxidans, M. tuberculosis, and other Gram-negative and acid-fast bacteria for the treatment of invasive infections, and especially antibacterial compounds that are highly soluble, remain active in vivo in the presence of serum and/or pulmonary surfactant, do not have hemolytic activity, and/or have a low propensity for resistance.

### SUMMARY OF THE DISCLOSURE

This application discloses novel methods of using phage lytic agents that are derived, for example, from Microviridae genomic sequences and are distinct from other such agents, including known lysins/artilysins and amurins (also referred to as Chlamydia phage (Chp) peptides). The application further discloses methods of using Chp peptides, lysins and polypeptide constructs comprising lysins and antimicrobial peptides (AMPs) that can be used, for example, to treat bacterial infections, including infections caused by Gram-negative bacteria, particularly multi-drug resistant and/or extensively drug resistant Gram-negative bacteria, including, but not limited to Pseudomonas aeruginosa (including mucoid, non-mucoid and/or hemolytic (such as α-hemolytic) isolates), Stenotrophomonas maltophilia, Achromobacter xylosoxidans, Achromobacter ruhlandii, Achromobacter dolens, and other highly-resistant bacterial pathogens known as ESKAPE pathogens (i.e., E. faecium, S. aureus, K. pneumoniae, A. baumannii, P. aeruginosa, and Enterobacter spp.), in another embodiment, Pseudomonas aeruginosa (Pseudomonoas aeruginosa (including mucoid, non-mucoid and/or hemolytic (such as α-hemolytic) isolates), Stenotrophomonas maltophilia, Achromobacter xylosoxidans, and other highly-resistant bacterial pathogens known as ESKAPE pathogens (i.e., E. faecium, S. aureus, K. pneumoniae, A. baumannii, P. aeruginosa, and Enterobacter spp.), in yet another embodiment, Pseudomonas aeruginosa and other highly-resistant bacterial pathogens known as ESKAPE pathogens (i.e., E. faecium, S. aureus, K. pneumoniae, A. baumannii, P. aeruginosa, and Enterobacter spp.).

In one aspect, the present disclosure is directed to a method of inhibiting the growth, reducing the population, or killing of at least one species of Gram-negative bacteria, the method comprising contacting the bacteria with a composition comprising an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity for a period of time sufficient to inhibit said growth, reduce said population, or kill said at least one species of Gram-negative bacteria.

In certain aspects of the methods of inhibiting the growth, reducing the population, or killing of at least one species of Gram-negative bacteria, the Chp peptide comprises an amino acid sequence selected from the group consisting of SEQ ID NO.2 (referred to herein as Chp2 or AM1), SEQ ID NO: 81 (referred to herein as Chp2-M1 or AM2), and SEQ ID NO. 89 (referred to herein as Chp10-M1 or AM3), and the at least one species of Gram-negative bacteria is selected from the group consisting of Achromobacter xylosoxidans, Achromobacter ruhlandii, Achromobacter dolens, Stenotrophomonas maltophilia and Pseudomonas aeruginosa (including mucoid, non-mucoid and/or hemolytic (such as α-hemolytic) isolates, MDR isolates, XDR isolates, and/or carbapenem-resistant isolates), in another embodiment, selected from Stenotrophomonas maltophilia and Pseudomonas aeruginosa, in still another embodiment, Achromobacter xylosoxidans, Achromobacter ruhlandii, Achromobacter dolens, and Stenotrophomonas maltophilia. In certain aspects, the lysin-AMP construct comprises the amino acid sequence of SEQ ID NO: 149 (referred to herein as GN370), and the at least one species of Gram-negative bacteria is selected from Klebsiella pneumoniae, Acinetobacter baumannii, Pseudomonas aeruginosa, Enterobacter cloacae, Escherichia coli, Citrobacter freundii, Enterobacter aerogenes, Klebsiella oxytoca, Kluyvera ascorbate, Raoultella ornithinolytica, and Salmonella senftenberg. In another embodiment of this aspect, the lysin-AMP construct comprises the amino acid sequence of SEQ ID NO: 149 (referred to herein as GN370), and the at least one species of Gram-negative bacteria is selected from Pseudomonoas aeruginosa (including mucoid, non-mucoid and/or hemolytic (such as α-hemolytic) isolates, MDR isolates, XDR isolates, and/or carbapenem-resistant isolates), Stenotrophomonas maltophilia, Achromobacter xylosoxidans and Achromobacter ruhlandii, in still another embodiment, Pseudomonoas aeruginosa (including mucoid, non-mucoid and/or hemolytic (such as α-hemolytic) isolates, MDR isolates, XDR isolates, and/or carbapenem-resistant isolates) and Stenotrophomonas maltophilia.

Also disclosed herein is a method of inhibiting a Gram-negative bacteria present in sputum comprising contacting the sputum with a composition comprising an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity for a period of time sufficient to inhibit said growth, reduce said population, or kill said at least one species of Gram-negative bacteria.

In certain aspects of the method of inhibiting a Gram-negative bacteria present in sputum, the Chp peptide comprises an amino acid sequence selected from the group consisting of SEQ ID NO. 2, SEQ ID NO: 81, and SEQ ID NO. 89, and the at least one species of Gram-negative bacteria is selected from the group consisting of Pseudomonas aeruginosa, Achromobacter xylosoxidans, and Stenotrophomonas maltophilia. In certain aspects, the lysin-AMP construct comprises the amino acid sequence of SEQ ID NO: 149, and the at least one species of Gram-negative bacteria is selected from the group consisting of Pseudomonas aeruginosa, Achromobacter xylosoxidans, and Stenotrophomonas maltophilia. In certain embodiments, the sputum is acquired from a patient with cystic fibrosis.

Further disclosed herein is a method of preventing, disrupting or eradicating a Gram-negative bacterial biofilm comprising administering (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, in an amount effective to kill at least one species of Gram-negative bacteria in the biofilm to a subject in need thereof.

In certain aspects of the methods of preventing, disrupting or eradicating a Gram-negative bacterial biofilm, the Chp peptide may comprise an amino acid sequence selected from the group consisting of SEQ ID NO. 2, SEQ ID NO: 81, and SEQ ID NO. 89, and the at least one species of Gram-negative bacteria is selected from Klebsiella pneumoniae, Acinetobacter baumannii, Pseudomonas aeruginosa, Enterobacter cloacae, Escherichia coli, Achromobacter xylosoxidans, Bulkholderia multivorans, and Stenotrophomonas maltophilia, in another embodiment, in addition to the foregoing bacteria, also selected from Achromobacter xylosoxidans and S. maltophilia. In certain aspects, the at least one species of Gram-negative bacteria, such as Pseudomonas aeruginosa, is a mucoid, non-mucoid and/or hemolytic (such as α-hemolytic) isolate, an MDR isolate, and/or an XDR isolate. In certain aspects of the methods of preventing, disrupting or eradicating a Gram-negative bacterial biofilm, the lysin-AMP construct comprises the amino acid sequence of SEQ ID NO: 149, and the at least one species of Gram-negative bacteria is selected from Klebsiella pneumoniae, Acinetobacter baumannii, Pseudomonas aeruginosa, Enterobacter cloacae, and Escherichia coli.

In yet another embodiment, there is disclosed a method of treating a bacterial infection caused by a Gram-negative bacteria, wherein the Gram-negative bacteria comprises Burkholderia cenocepacia and optionally one or more additional species of Gram-negative bacteria, which method comprises administering to a subject diagnosed with, at risk for, or exhibiting symptoms of a bacterial infection, a pharmaceutical composition containing an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP having at least 80% sequence identity with the amino acid sequence selected from the group consisting of SEQ ID NOs. 1-26, 54-67, 81-102, and 129-161, said Chp peptide, lysin, or lysin-AMP construct having lytic activity, wherein the composition comprises at least one activity selected from inhibiting Burkholderia cenocepacia bacterial growth, reducing a Burkholderia cenocepacia bacterial population and/or killing Burkholderia cenocepacia.

In certain aspects of the methods of treating a bacterial infection caused by a Gram-negative bacteria, the lysin-AMP construct comprises the amino acid sequence of SEQ ID NO: 149.

In any of the foregoing methods/uses, the at least one species of Gram-negative bacteria may be selected from the group consisting of Klebsiella pneumoniae, Acinetobacter baumannii, Pseudomonas aeruginosa, Enterobacter cloacae, Escherichia coli, Achromobacter xylosoxidans, Achromobacter ruhlandii, Achromobacter dolens, Bulkholderia multivorans, Burkholderia cenocepacia, Burkholderia cepacia, Pandoraea apista, Stenotrophomonas maltophilia, Ralstonia mannitolilytica, Serratia marcescens, Citrobacter freundii, Enterobacter aerogenes, Klebsiella oxytoca, Kluyvera ascorbate, Raoultella ornithinolytica, and Salmonella senftenberg. In certain aspects, the at least one species of Gram-negative bacteria, such as Pseudomonas aeruginosa, is a mucoid, non-mucoid and/or hemolytic (such as α-hemolytic) isolate, an MDR isolate, and/or a XDR isolate.

In some embodiments disclosed herein, the at least one species of Gram-negative bacteria further comprises Pseudomonas aeruginosa, including, for example, mucoid, non-mucoid and/or hemolytic (such as α-hemolytic) isolates, MDR isolates, and/or XDR isolates of Pseudomonas aeruginosa. In certain embodiments, the bacterial infection is a topical or systemic bacterial infection. In certain embodiments, the bacterial infection is present in a subject with cystic fibrosis, for example in the lungs of the cystic fibrosis patient. In another embodiment, the bacterial infection is in cystic fibrosis (CF) patients and/or patients associated with pulmonary exacerbation (PEx) and/or decline in lung function and mortality. In yet another embodiment, the bacterial infection is in non-CF bronchiectasis and/or potentially acute pneumonias. Therefore, contemplated herein are methods of treating cystic fibrosis comprising administering to a subject in need thereof an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP having at least 80% sequence identity with the amino acid sequence selected from the group consisting of SEQ ID NOs. 1-26, 54-67, 81-102, and 129-161, said Chp peptide, lysin, or lysin-AMP construct having lytic activity.

In some embodiments, any of the foregoing methods/uses further comprises administering to the subject an antibiotic suitable for the treatment of a Gram-negative bacterial infection. In certain embodiments, the antibiotic suitable for the treatment of Gram-negative bacterial infection is selected from one or more of a cephalosporin (such as cefataxime, ceftriaxone, ceftazidime, cefepime, cefoperazone, ceftobiprole, and cefazolin), a tetracycline (such as minocycline), tigecycline, trimethoprim, sulfamethoxazole, ciprofloxacin, levofloxacin, an aminoglycoside (such as gentamicin, tobramycin, and amikacin), a carbapenem (such as imipenem, meropenem, and doripenem), a penicillin (such as piperacillin, ampicillin and ticarcillin), rifampicin, polymyxin B, and colistin. In certain embodiments, the antibiotic is selected from one or more of amikacin, azithromycin, aztreonam, ciprofloxacin, colistin, fosfomycin, gentamicin, imipenem, piperacillin, rifampicin, and tobramycin. In some embodiments, the antibiotic suitable for the treatment of acid-fast bacterial infection is selected from one or more of isoniazid, rifampin, ethambutol, and pyrazinamide. In certain embodiments, the antibiotic is selected from meropenem.

Further disclosed herein is a method of suppressing the ability of a Gram-negative bacteria to develop resistance to an antibiotic comprising administering to the Gram-negative bacteria (a) the antibiotic; and (b) (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, in an amount effective to suppress the Gram-negative bacteria's development of resistance to the antibiotic. In certain embodiments of the method disclosed herein, the lysin-AMP construct comprises the amino acid sequence of GN370 (SEQ ID NO: 149). In certain embodiments, the antibiotic is a fluoroquinolone (optionally levofloxacin), a carbapenem (optionally meropenem), or an aminoglycoside (optionally tobramycin). In certain embodiments, the Chp peptide, lysin, or lysin-AMP construct (optionally GN370) is administered in a sub-MIC amount to the Gram-negative bacteria, such as ⅛^{th} of the MIC, 1/16^{th} of the MIC, or ⅓^{nd} of the MIC.

In certain aspects of any of the foregoing methods/uses, the Chp peptide, lysin, or lysin-AMP construct has no or a low propensity towards developing resistance towards a Gram-negative bacteria. In certain aspects of any of the foregoing methods/uses, the bacteria is multidrug or extensively drug-resistant.

In further embodiments, there is disclosed a method of treating a bacterial infection caused by a Gram-negative bacteria, which method comprises administering to a subject in need thereof, a pharmaceutical composition containing an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, wherein the composition comprises at least one activity selected from inhibiting, reducing and/or killing the growth of said Gram negative bacteria.

In certain embodiments, the infection is in cystic fibrosis (CF) patients and/or patients associated with pulmonary exacerbation (PEx) and/or decline in lung function and mortality, and in certain embodiments, the infection is non-CF bronchiectasis and/or potentially acute pneumonias. According to certain embodiments disclosed herein, the at least one species of Gram-negative bacteria is mycobacterium species, e.g., selected from M. smegmatis, M. fortuitum, M. avium, M. kansaii, M. scrofulaceum, M. intracellulare and M. tuberculosis, and in certain embodiments, the at least one species of Gram-negative bacteria is M. abscessus.

### BRIEF DESCRIPTION OF THE DRAWING

FIG. 1 are three-dimensional models predicted by I-Tasser for structures of Chlamydia phage peptide (Chp) family members Chp1, Chp 2, Chp4, Chp5, Chp6, Chp7, Ecp1, Ecp2, and Osp1. The human innate immune effector peptide LL-37 is included for comparison. Alpha helical structures are evident, and the top terminal is generally the N-terminal.

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log 10 (99.9%) or better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on any and all combinations of Gram-positive bacteria, Gram-negative bacteria, acid-fast bacteria, and non-acid fast bacteria. By way of example, an antibiotic can affect cell wall peptidoglycan biosynthesis, cell membrane integrity, or DNA or protein synthesis in bacteria. Nonlimiting examples of antibiotics active against Gram-negative bacteria include cephalosporins, such as ceftriaxone-cefotaxime, ceftazidime, cefepime, cefoperazone, and ceftobiprole; fluoroquinolones such as ciprofloxacin and levofloxacin; aminoglycosides such as gentamicin, tobramycin, and amikacin; piperacillin, ticarcillin, imipenem, meropenem, doripenem, broad spectrum penicillins with or without beta-lactamase inhibitors, rifampicin, polymyxin B, and colistin. Non-limiting examples of antibiotics active against acid-fast bacteria include isoniazid, rifampin, ethambutol, and pyrazinamide.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). An “extensively drug resistant” or “extremely drug resistant” (“XDR”) pathogen is one that has developed resistance to at least one agent in all but two or fewer antimicrobial classes such that the pathogen remains susceptible to only one or two classes. Antimicrobial classes or categories may be as defined, for example, in Magiorakos, A. P., Multidrug-resistant, extensively drug-resistant and pandrug-resistant bacteria: an international expert proposal for interim standard definitions for acquired resistance, Clin. Microbiol. Infect. 2012; 18:268-281. In certain embodiments, the antimicrobial classes of agents used against P. aeruginosa may be found in Table B as set forth herein. One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, Gram-negative or acid-fast bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a Chp peptide or a lysin or a lysin-AMP construct and an antibiotic or any other antibacterial agent, in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of Chp peptides, lysins, or lysin-AMP constructs with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, a Chp peptide or a lysin or lysin-AMP construct could be administered only initially within 24 hours of an additional antibiotic, and then the additional antibiotic use may continue without further administration of the Chp peptide, lysin, or lysin-AMP construct.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive, Gram-negative bacterial infections, or acid-fast bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-negative or acid-fast bacteria, whether such infection be systemic, topical or otherwise concentrated or confined to a particular organ or tissue.

“Polypeptide” is used herein interchangeably with the term “peptide” and refers to a polymer made from amino acid residues and generally having at least about 30 amino acid residues. The term includes not only polypeptides in isolated form, but also active fragments and derivatives thereof, including modified variants. The term “polypeptide” also encompasses fusion proteins or fusion polypeptides comprising a Chp peptide, lysin, and/or lysin-AMP construct as described herein and maintaining, for example a lytic function. Depending on context, a polypeptide can be a naturally occurring polypeptide or a recombinant, engineered, or synthetically produced polypeptide. A particular Chp peptide, lysin, or lysin-AMP construct can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining, e.g., lytic activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” may also refer to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” The open-ended expression “a polypeptide comprising” a certain structure includes larger molecules than the recited structure, such as fusion polypeptides.

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a Chp peptide, lysin, lysin-AMP fragment or active fragment thereof and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more Chp peptides or active fragments thereof. These can be used to make a fusion polypeptide with lytic activity.

“Active fragment” refers to a portion of a polypeptide that retains one or more functions or biological activities of the isolated polypeptide from which the fragment was taken, for example bactericidal activity against one or more Gram-negative or acid-fast bacteria.

“Amphipathic peptide” refers to a peptide having both hydrophilic and hydrophobic functional groups. In certain embodiments, secondary structure may place hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides that are synthetically produced peptides composed of mostly positively charged amino acid residues, such as lysine (Lys) and/or arginine (Arg) residues. The amino acid residues that are not positively charged can be neutrally charged amino acid residues, negatively charged amino acid residues, and/or hydrophobic amino acid residues.

“Hydrophobic group” refers to a chemical group such as an amino acid side chain that has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of a bacteria in the subject and thereby controlling or reducing a bacterial infection in a subject or bacterial contamination of an organ, tissue, or environment. Thus “treatment” that reduces incidence may, for example, be effective to inhibit growth of at least one Gram-negative or acid-fast bacterium in a particular milieu, whether it be a subject or an environment. On the other hand, “treatment” of an already established infection refers to inhibiting the growth, reducing the population, killing, including eradicating, a Gram-negative bacteria and/or an acid-fast bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitute examples of prevention.

“Contracted diseases” refers to diseases manifesting with clinical or subclinical symptoms, such as the detection of fever, sepsis, or bacteremia, as well as diseases that may be detected by growth of a bacterial pathogen (e.g., in culture) when symptoms associated with such pathology are not yet manifest.

The term “derivative” in the context of a peptide or polypeptide or active fragments thereof is intended to encompass, for example, a polypeptide modified to contain one or more chemical moieties other than an amino acid that do not substantially adversely impact or destroy the lytic activity. The chemical moiety can be linked covalently to the peptide, e.g., via an amino terminal amino acid residue, a carboxy terminal amino acid residue, or at an internal amino acid residue.

Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to peptides include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a peptide or fused to a peptide without interfering with normal functions of cellular proteins. In certain embodiments, a polynucleotide encoding a fluorescent protein may be inserted upstream or downstream of the polynucleotide sequence. This will produce a fusion protein (e.g., Chp Peptide::GFP) that does not interfere with cellular function or function of a peptide to which it is attached. Polyethylene glycol (PEG) conjugation to proteins has been used as a method for extending the circulating half-life of many pharmaceutical proteins. Thus, in the context of peptide derivatives, the term “derivative” encompasses peptides chemically modified by covalent attachment of one or more PEG molecules. It is anticipated that pegylated peptides will exhibit prolonged circulation half-life compared to the unpegylated peptides, while retaining biological and therapeutic activity.

“Modified variant” refers to a Chp peptide, lysin, or lysin-AMP construct wherein a non-naturally occurring modification has been made to the amino acid sequence that either enhances the lytic activity or does not substantially adversely impact or destroy the lytic activity of the Chp peptide, lysin, or lysin-AMP construct. Exemplary modifications that may be made to modified variants include modifying an amino acid of the Chp peptide, such as a positively charged amino acid, from an L-form to a D-form; adding an amino acid residue or residues to the C-terminus and/or the N-terminus, forming fusion polypeptides, and forming charge array variants, wherein amino acid charges have been reordered.

“Percent amino acid sequence identity” refers to the percentage of amino acid residues in a candidate sequence that are identical with the amino acid residues in the reference polypeptide sequence, such as a specific Chp peptide, lysin, or lysin-AMP construct polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to peptides as well. Thus, the term “substantially identical” will encompass mutated, truncated, fused, or otherwise sequence-modified forms of isolated polypeptides and peptides described herein, and active fragments thereof, as well as polypeptides with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide.

As used herein, two amino acid sequences are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides of the present disclosure are substantially homologous when one or more, such as up to 10%, up to 15%, or up to 20% of the amino acids of the polypeptide, such as the Chp peptides, lysin, or lysin-AMP constructs described herein, are substituted with a similar or conservative amino acid substitution, and wherein the resulting peptides have at least one activity (e.g., antibacterial effect) and/or bacterial specificities of the reference polypeptide, such as the Chp peptides, lysin, or lysin-AMP constructs disclosed herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Preventing biofilm formation” refers to the prevention of the incidence, recurrence, spread, onset or establishment of a biofilm. It is not intended that the present disclosure be limited to complete prevention or to prevention of establishment of biofilm. In some embodiments, the onset of a biofilm is delayed, or the establishment of a biofilm is reduced or the chance of formation of a new biofilm is reduced, and such constitute examples of prevention of a biofilm. Further, prevention of a biofilm may be due to any mechanism including 1) effectively killing planktonic bacteria; 2) killing “persister” bacterial cells in suspensions, i.e., bacteria that are metabolically inactive, tolerant of antibiotics, and highly associated with biofilm formation; and/or 3) preventing “aggregation”, i.e., the ability of bacteria to attach to one another via proteins or polysaccharides.

“Eradication” in reference to a biofilm includes 1) effectively killing bacteria in a biofilm including persister bacterial cells in the biofilm and, optionally 2) effectively destroying and/or damaging the biofilm matrix.

“Disruption” in reference to a biofilm refers to a mechanism that falls between prevention and eradication. A biofilm, which is disrupted, may be “opened”, or otherwise damaged, thus permitting, e.g., an antibiotic, to more readily penetrate the biofilm and kill the bacteria.

“Suitable” in the context of an antibiotic being suitable for use against certain bacteria refers to an antibiotic that was found to be effective against those bacteria even if resistance subsequently developed.

“Outer Membrane” or “OM” refers to a feature of Gram-negative bacteria. The outer membrane is comprised of a lipid bilayer with an internal leaflet of phospholipids and an external amphiphilic leaflet largely consisting of lipopolysaccharide (LPS). The LPS has three main sections: a hexa-acylated glucosamine-based phospholipid called lipid A, a polysaccharide core and an extended, external polysaccharide chain called 0-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the peptidoglycan forms a thin layer that is very sensitive to hydrolytic cleavage—unlike the peptidoglycan of Gram-negative bacteria which is 30-100 nanometers (nm) thick and consists of up to 40 layers, the peptidoglycan of Gram-negative bacteria is only 2-3 nm thick and consists of only 1-3 layers.

### Polypeptides

### Amurins

This application discloses methods of using phage lytic agents that are derived, for example, from Microviridae genomic sequences and are distinct from other such agents, including known lysins/artilysins and amurins. The phage lytic agents disclosed herein are referred to as Chlamydia phage (Chp) peptides, also referred to as “amurin peptides” (a functional definition not implying sequence similarity with amurins). Disclosed herein are various Chp peptides that have been identified, constituting a family of specific bacteriolytic proteins, as well as non-naturally occurring modified variants of those Chp peptides (corresponding to SEQ ID NOs. 81-91 and 94-102).

As used herein, “Chp peptides” refers to both naturally-occurring Chp peptides, non-naturally occurring modified variants thereof, and modified Chp peptides having at least one modification (e.g., substitution) as compared to a wild-type Chp peptide. Several of the Chp peptides disclosed herein exhibit notable sequence similarities to each other but are distinct from other known peptides in the sequence databases. Despite the unique sequences of the Chp peptides, they are all predicted to adopt alpha-helical structures similar to some previously described antimicrobial peptides (AMPs) of vertebrate innate immune systems (E. F. Haney et al, 2017, In Hansen P R (ed), Antimicrobial Peptides: Methods and Protocols, Methods in Molecular Biology, vol. 1548) but with no sequence similarity to such AMPs. Consistent with an antibacterial function for the Chp class, disclosed herein is the potent and broad-spectrum bactericidal activity against Gram-negative and acid-fast pathogens for several different purified Chp peptides. Unlike the previously described amurins of Microviridae, which have cytoplasmic targets in the cell wall biosynthetic apparatus that may not be easily accessed by externally applied proteins, the Chp peptides disclosed herein can be used, in purified forms, to exert bactericidal activity “from without,” i.e., by acting on the outside of the cell wall. The Chp peptides identified here represent a novel class of antimicrobial agents having broad-spectrum activity against Gram-negative and acid-fast pathogens and the ability to persist in the presence of serum and/or pulmonary surfactant. Exemplary Chp peptides are described, for instance, in PCT Publication No. WO 2021/007107, incorporate by reference herein in its entirety. As demonstrated and explained herein, the Chp peptides described in this section, including wild-type Chp peptides, modified Chp peptides, derivatives, modified variants, or active fragments thereof, can be used in the pharmaceutical compositions and methods described herein.

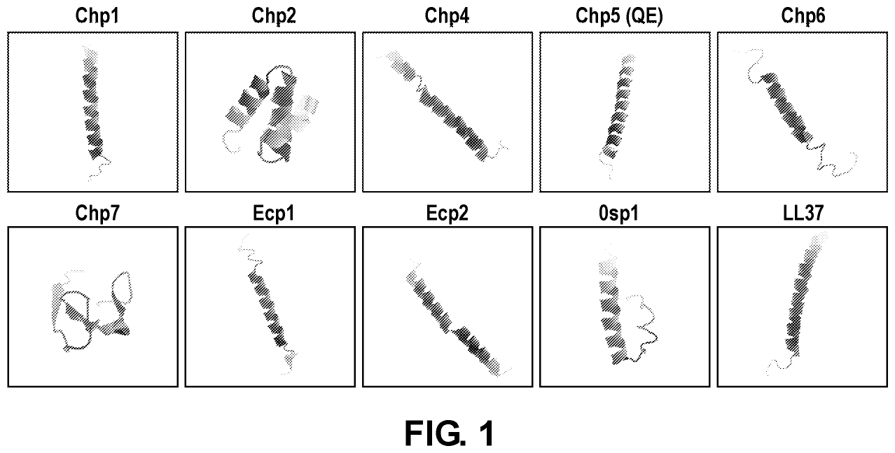

In some embodiments, the α-helix domain spans most of the molecule. See, e.g., Chp1 and Chp4 in FIG. 1. In some embodiments, the α-helix domain is interrupted (see, e.g., Chp2 in FIG. 1), and in some embodiments, the α-helix domain is truncated (see, e.g., Chp6 and Osp1 in FIG. 1). The α-helix domain of the Chp peptides of the present disclosure varies in size between about 3 and 32 amino acids, more typically between about 10 and 25 amino acid residues.

The modified Chp peptides of the present disclosure typically retain one or more functional or biological activities of the reference Chp peptide. In some embodiments, the modification improves the antibacterial activity of the Chp peptide. Typically, the modified Chp peptide has improved in vitro antibacterial activity (e.g., in buffer and/or media) in comparison to the reference Chp peptide. In other embodiments, the modified Chp peptide has improved in vivo antibacterial activity (e.g., in an animal infection model). In some embodiments, the modification improves the antibacterial activity of the Chp peptide in the absence and/or presence of human serum. In some embodiments, the modification improves the antibacterial activity of the Chp peptide in the absence and/or presence of pulmonary surfactant.

In some embodiments, Chp peptides disclosed herein or variants or active fragments thereof are capable of inhibiting the growth of, or reducing the population of, or killing P. aeruginosa and/or at least one species of acid-fast bacteria, such as M. tuberculosis, and, optionally, at least one other species of Gram-negative or acid-fast bacteria in the absence or presence of, or in both the absence and presence of, human serum. In some embodiments, Chp peptides disclosed herein or variants or active fragments thereof are capable of inhibiting the growth of, or reducing the population of, or killing P. aeruginosa and/or at least one species of acid-fast bacteria, such as M. tuberculosis, and, optionally, at least one other species of Gram-negative or acid-fast bacteria in the absence or presence of, or in both the absence and presence of pulmonary surfactant.

In certain embodiments, the modified Chp peptide comprises a polypeptide sequence having at least 80%, such as at least 85%, such as at least 90%, such as at least 92.5%, such as at least 95%, such as at least 98%, or such as at least 99% sequence identity with the amino acid sequence of at least one Chp peptide selected from the group consisting of SEQ ID NOs. 1-4, 6-26, 54-66, 81-91 and 94-102 or an active fragment thereof, wherein the modified Chp peptide inhibits the growth, reduces the population, and/or kills at least one species of Gram-negative bacteria, such as P. aeruginosa, or at least one species of acid-fast bacteria, such as actinobacteria, including mycobacteria, and optionally at least one additional species of Gram-negative or acid-fast bacteria as described herein, optionally in the presence of human serum and/or pulmonary surfactant.

In some embodiments, the Chp peptide of the present disclosure is a derivative of one of the reference Chp peptides that has been chemically modified. A chemical modification includes but is not limited to, adding chemical moieties, creating new bonds, and removing chemical moieties. Chemical modifications can occur anywhere in a Chp peptide, including the amino acid side chains, as well as the amino or carboxyl termini. For example, in certain embodiments, the Chp peptide comprises an N-terminal acetylation modification. In certain embodiments, the Chp peptide or active fragment thereof comprises a C-terminal amidation modification. Such modifications can be present at more than one site in a Chp peptide.

Furthermore, one or more side groups, or terminal groups of a Chp peptide or active fragment thereof may be protected by protective groups known to the person ordinarily-skilled in the art.

In some embodiments, the Chp peptides or active fragments thereof are conjugated to a duration enhancing moiety. In some embodiments, the duration enhancing moiety is polyethylene glycol. Polyethylene glycol (“PEG”) has been used to obtain therapeutic polypeptides of enhanced duration (Zalipsky, S., Bioconjugate Chemistry, 6:150-165 (1995); Mehvar, R., J. Pharm. Pharmaceut. Sci., 3:125-136 (2000), which is herein incorporated by reference in its entirety). The PEG backbone, (CH₂CH₂—O—)ₙ, wherein n is a number of repeating monomers, is flexible and amphiphilic. When attached to another chemical entity, such as a Chp peptide or active fragment thereof, PEG polymer chains can protect such polypeptides from immune response and other clearance mechanisms. As a result, pegylation can lead to improved efficacy and safety by optimizing pharmacokinetics, increasing bioavailability, and decreasing immunogenicity and dosing amount and/or frequency.

In certain embodiments, the Chp peptide is a modified variant wherein the positive amino acids (arginine, lysine, and histidine), which naturally appear in their L-isoform, have been replaced by the same amino acid in the D-isoform. It has been shown with a different antimicrobial protein derived from sapesin B that variants containing D-isoform amino acids may exhibit higher antimicrobial activity. See, e.g., Manabe et al., Scientific Reports (2017); DOI:10.1038/srep43384. In certain embodiments, the Chp peptide is a modified variant wherein an amino acid residue or residues have been added to the C-terminus, the N-terminus, or both the C-terminus and the N-terminus. For example, in certain embodiments, a cysteine may be added to the C-terminus and/or the N-terminus. In certain embodiments, residues that are known to confer stability to alpha-helices and/or to promote activity in the presence of salt may be added to the C-terminus and/or the N-terminus. See, e.g., Park et al., Helix stability confers salt resistance upon helical antimicrobial peptides, J. Biol. Chem. (2004); 279(14):13896-901. In yet further embodiments, the Chp peptide is a modified variant that is a charge array variant, wherein the amino acids have been reordered based on their charges to maintain amphipathic helical structures. In still further embodiments, the amino acid residues may be scrambled to create the modified variant which may, in certain embodiments, act as a control peptide.

In some embodiments, the Chp peptides disclosed herein and active fragments thereof are capable of penetrating the outer membrane of Gram-negative bacteria. Without being limited by theory, after penetration of the outer membrane, the Chp peptides or active fragments thereof can degrade peptidoglycan, a major structural component of the bacterial cell wall, resulting in cell lysis. In some embodiments, the Chp peptides or active fragments thereof disclosed herein contain positively charged (and amphipathic)N- and/or C-terminal α-helical domains that facilitate binding to the anionic outer membrane of a Gram-negative bacteria to effect translocation into the sub-adjacent peptidoglycan.

The ability of a Chp peptide or active fragment thereof to penetrate an outer membrane of a Gram-negative bacteria may be assessed by any method known in the art, such as described in WO 2017/049233, which is herein incorporated by reference in its entirety. For example, the Chp peptide or active fragment thereof may be incubated with Gram-negative bacteria and a hydrophobic compound. Most Gram-negative bacteria are strongly resistant to hydrophobic compounds, due to the presence of the outer membrane and, thus, do not allow the uptake of hydrophobic agents such as 1-N-phenylnaphthylamine (NPN), crystal violet, or 8-anilino-1-naphthalenesulfonic acid (ANS). NPN is largely excluded by intact Gram-negative bacteria, but enhanced uptake of NPN may occur in cells having a damaged or permeable outer membrane. NPN fluoresces strongly under hydrophobic conditions and weakly under aqueous conditions. Therefore, NPN's interaction with membrane phospholipids in the bacterial envelope increases its fluorescence signal and can be used as an indication of a compromised bacterial membrane and a measurement of the outer membrane permeability.

More particularly, the ability of a Chp peptide or active fragment thereof to penetrate an outer wall may be assessed by incubating, e.g., NPN with a Gram-negative bacteria, e.g., P. aeruginosa strain PAO1, in the presence of the Chp peptide or active fragment thereof to be tested for activity. A higher induction of fluorescence in comparison to the fluorescence emitted in the absence of a Chp peptide (negative control) indicates outer membrane penetration. In addition, fluorescence induction can be compared to that of established permeabilizing agents, such as EDTA (ethylene diamine tetraacetate) or an antibiotic such as an antibiotic of last resort used in the treatment of P. aeruginosa, i.e., Polymyxin B (PMB) to assess the level of outer membrane permeabilization.

Multiple protocols throughout the literature detail various method of action studies using NPN and amurin peptides, such as, for example, (1) Mohamed et al., A short D-enantiomeric antimicrobial peptide with potent immunomodulatory and antibiofilm activity against multidrug-resistant Pseudomonas aeruginosa and Acinetobacter baumannii, SCIENTIFIC REPORTS 2017; 6953(7):1-13; (2) Lv et al., Antimicrobial Properties and Membrane-Active Mechanism of a Potential a-Helical Antimicrobial Derived from Cathelicidin PMAP-36, PLoS One 2014; 9:e86364; (3) Wang et al., High specific selectivity and Membrane-Active Mechanism of the synthetic centrosymmetric a-helical peptides with Gly-Gly pairs, SCIENTIFIC REPORTS 2015; 15963(5):1-19; and (4) Shao et al., Symmetrical Modification of Minimized Dermaseptins to Extend the Spectrum of Antimicrobials with Endotoxin Neutralization Potency, INTERNATIONAL J. MOL. SCI. 2019; 1417(20). Accordingly, based on the literature, certain control peptides may be used and compared to the Chp peptides disclosed herein. For example, Lv et al. 2014 discloses melittin, a bee venom peptide that causes rapid (e.g., within minutes) membrane disruption and a fast increase in NPN uptake. Wang et al. 2015 discloses LL-37, a human innate immune molecule that causes membrane disruption and uptake of NPN. Additionally, colistin, a known peptide having potent membrane disruption activity, and charged-reversed variants of the Chp peptides disclosed herein, such as Chp5, may be used as controls, for example in furtherance of the study of the mechanism of action of the Chp peptides disclosed herein.

The ability of a Chp peptide to disrupt the outer membrane of Gram-negative bacteria may be assessed, for example, by measuring the EC₅₀, or the concentration where the bacterial sample had 50% maximal incorporation of NPN into the outer membrane at 10 minutes. When the Chp peptides Chp2, Chp2-M1, and Chp10-M1 were tested for their ability to permeabilize the outer membrane of Gram-negative bacteria (including P. aeruginosa, E. cloacae, K. pneumoniae, E. coli, and A. baumannii), a dose-dependent increase in outer membrane permeability, equivalent to that of colistin, LL37, and melittin, was observed. Thus, in certain embodiments, when Gram-negative bacteria is contacted with a Chp peptide as disclosed herein, the Gram-negative bacteria may exhibit an EC₅₀ comparable to or less than the EC₅₀ of the Gram-negative bacteria exposed to a control peptide, such as colistin, LL-37, or melittin, indicating the Chp peptides disclosed herein allow for increased NPN uptake and percent permeabilization of the outer membrane of Gram-negative bacteria, including P. aeruginosa, E. cloacae, K. pneumoniae, E. coli, and A. baumannii.

The mechanism of action of Chp peptides disclosed herein can also be evaluated by measuring the depolarization of the inner membrane of Gram-negative bacteria, including MDR and XDR strains. The inner membrane comprises hydroxylated phospholipids such as cardiolipin, phosphatidylglycerol, and phosphatidylserine. This creates a net negative charge at physiological pH, which is believed to enhance the binding of cationic peptides, including the Chp peptides disclosed herein. Upon permeabilization of the outer membrane, the ability of Chp peptides to induce dissipation of the cytoplasmic membrane electrical potential gradient (Ay) may be examined, for example by following the release of 3,3′-dipropylthiadicarbocyanine (diSC₃-5) as a function of time compared to an untreated control. DiSC₃-5 is a fluorophore that is a caged cation concentrated within the bacterial inner membrane and under the influence of the bacterial membrane electrical potential gradient. At high concentrations, diSC₃-5 self-quenches, leading to the suppression of fluorescence. When the inner membrane deteriorates or becomes leaky for cations, including protons, the Δψ dissipates, which leads to a release of diSC₃-5 and a subsequent increase in fluorescence. Control peptides such as LL-37 and melittin have been shown to dissipate Ay, and may be used as a comparison to evaluate the dissipation potential of Chp peptides disclosed herein in various Gram-negative bacteria. When the Chp peptides Chp2, Chp2-M1, and Chp10-M1 were tested for their ability to depolarize the inner membrane of Gram-negative bacteria (including P. aeruginosa, E. cloacae, K. pneumoniae, E. coli, and A. baumannii), a dose-dependent increase in membrane depolarization, equivalent or superior to that of melittin, LL37, RI-18, and PMBN, was observed. Without wishing to be bound by theory, it is believed that the adsorption and binding of Chp peptides to the inner membrane and insertion into the lipid bilayer results in membrane permeabilization and pore/ion channel formation, which is concomitant with the collapse of the membrane's electrical potential.

The damage caused by the Chp peptides disclosed herein to the outer and inner membranes of Gram-negative bacteria, including MDR and XDR strains, can also be assessed with impermeable dyes such as propidium iodide. Protocols for assessing the ability of propidium iodide to cross a bacterial membrane that has been damaged by amurin peptides, intercalate into DNA, and emit a fluorescent signal are known in the art, including, for example, in Mohamed et al. 2017; Wang et al. 2015; Kwon et al., Mechanism of action of antimicrobial peptide P5 truncation against Pseudomonas aeruginosa and Staphyloccus aureus, AMB ExPRESS 2019; 9:122; and Nagant et al., Identification of Peptides Derived from the Human Antimicrobial Peptide LL-37 Active against Biofilms Formed by Pseudomonas aeruginosa Using a Library of Truncated Fragments, ANTIMICROB. AGENTS CHEMO. 2012; 56:5698-5708. As with NPN, the EC₅₀ of propidium iodide may be measured in Gram-negative bacteria contacted with a Chp peptide after a given time and compared to the EC₅₀ of Gram-negative bacteria that is untreated or that has been contacted, for example, with a control peptide such as colistin, LL-37, or melittin. When the Chp peptides Chp2, Chp2-M1, and Chp10-M1 were tested for their ability to enhance the uptake of propidium iodide in Gram-negative bacteria (including P. aeruginosa, E. cloacae, K. pneumoniae, E. coli, and A. baumannii), a dose-dependent increase in cell envelop permeability was equivalent to that of colistin, LL37, and melittin.

Additionally, the mechanism of action of both the Chp peptides and the lysins and/or lysin-AMP constructs disclosed herein may be evaluated through the use of Scanning Electron Microscopy (SEM) and Transmission Electron Microscopy (TEM) techniques. Due to the rapid permeabilization and depolarization of bacterial membranes when contacted with Chp peptides, as discussed above, membrane damage may be visualized through SEM and TEM. SEM and TEM images demonstrate that the Chp peptides disclosed herein, as with many known lysins, may be marked by the appearance of “membrane bubbles,” followed by lysis of the membrane. TEM and SEM images taken approximately 2 minutes after Chp peptide (8 μg/mL) contact with P. aeruginosa show the formation of multiple bulges on the bacterial membrane, as well as the appearance of pore formation and degradation of the cellular membrane. TEM and SEM images taken approximately 5 minutes after Chp peptide (8 μg/mL) contact with P. aeruginosa show continued formation of membrane bulges, along with degradation of the cellular membrane, condensation of electron-dense cytoplasmic material, formation of spheroplasts, and pore formation. TEM and SEM images taken approximately 20 minutes after Chp peptide (8 μg/mL) contact with P. aeruginosa show multiple instances of pore formation and the appearance of ghost cells, empty of intracellular content. The GN370 lysin-AMP construct caused more extensive and larger membrane blebs, with the appearance of amorphous forms (i.e., loss of the rod-shaped form), prior to pore formation and lysis, while the amurins resulted in more discrete and smaller regions of membrane destabilization prior to the appearance of pores and lysis. Taken together, the TEM and SEM images demonstrate that membrane lysis due to contact with Chp peptides and the lysins and/or lysin-AMP constructs disclosed herein may occur through a three-step process comprising membrane bubbling or bulging, pore formation, and cell lysis, resulting in the release of a filamentous material.

In some embodiments, the Chp peptides disclosed herein or active fragments thereof exhibit lytic activity in the presence and/or absence of human serum. Suitable methods for assessing the activity of a Chp peptide or active fragment thereof in human serum are known in the art and described in the examples. Briefly, a MIC value (i.e., the minimum concentration of peptide sufficient to suppress at least 80% of the bacterial growth compared to control) may be determined for a Chp peptide or active fragment thereof and compared to, e.g., a compound inactive in human serum, e.g., T4 phage lysozyme or artilysin GN126. T4 phage lysozyme is commercially available, e.g. from Sigma-Aldrich, Inc. GN126 corresponds to Art-175, which is described in the literature and is obtained by fusing AMP SMAP-29 to GN lysin KZ144. See Briers et al. 2014, Antimicrob, Agents Chemother. 58:3774-3784, which is herein incorporated by reference in its entirety.

In some embodiments, the Chp peptides disclosed herein or active fragments thereof exhibit lytic activity in the presence and/or absence of pulmonary surfactant. Suitable methods for assessing the activity of a Chp peptide or active fragment thereof in pulmonary surfactant are known in the art and described in the examples. As with for assessing the activity in human serum, a MIC value may be determined for a Chp peptide or active fragment thereof in pulmonary surfactant or a suitable substitute (e.g., Survanta®) and optionally compared to a compound exhibiting reduced activity in pulmonary surfactant and/or Survanta®, such as daptomycin.

More particularly MIC values for a Chp peptide or active fragment thereof may be determined against particular bacteria, including e.g., the laboratory P. aeruginosa strains PA01 and CFS-1292, in various standard and non-standard media, e.g., Mueller-Hinton broth (MHB), MHB supplemented with human serum or Survanta®, MHB without starch (MHBns), CAA as described herein, which includes physiological salt concentrations, CAA supplemented with human serum or Survanta®, CAA supplemented with Tween 80®, e.g., 0.002% Tween 80® (CAA_{T}), CAA_{T} supplemented with starch or beef extract, modified RPMI, Dulbecco's Modified Eagle Medium (DMEM), and tryptic soy broth. The use of PA01 enables testing in the presence of elevated serum concentrations since unlike most clinical isolates, PA01 is insensitive to the antibacterial activity of human blood matrices. Other bacteria may also be used to determine MIC values for a Chp peptide or active fragment thereof including, e.g., the laboratory strain Mycobacterium smegmatis MC²155; attenuated Mycobacterium tuberculosis (Zopf) Lehmann and Neumann ATCC® Strains 35818, 25177, 35817, and 35818; and non-tuberculosis mycobacterium strains, including, for example, Mycobacterium avium strain Chester (ATCC® 700898), Mycobacterium kansasii strain Hauduroy (ATCC® 12478), Mycobacterium scrofulaceum strain Prissick and Masson (ATCC® 19981), Mycobacterium peregrinum strain Kusunoki and Ezaki (ATCC® 700686), Mycobacterium marinum strain Aronson (ATCC® 927), Mycobacterium intracellulare strain (Cuttino and McCabe) Runyon (ATCC® 13950), and Mycobacterium fortuitum subspecies fortuitum da Costa Cruz (ATCC® 6841).

In some embodiments, the Chp peptides disclosed herein or active fragments thereof are capable of reducing a biofilm. Methods for assessing the Minimal Biofilm Eradicating Concentration (MBEC) of a Chp peptide or active fragment thereof may be determined using a variation of the broth microdilution MIC method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method, fresh colonies of e.g., a P. aeruginosa strain, such as ATCC 17647, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; lnnovotech Inc.) and incubated e.g., 24 hours at 37° C. Biofilms are then washed and treated with e.g., a 2-fold dilution series of the lysin in TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum Chp peptide concentration required to remove at least 95% of the biofilm biomass assessed by crystal violet quantitation.

In some embodiments, the Chp peptides disclosed herein or active fragments thereof reduce the minimum inhibitory concentration (MIC) of an antibiotic in the presence and/or absence of human serum, pulmonary surfactant, and/or sputum, including human sputum and human sputum from a cystic fibrosis patient. Any known method to assess MIC may be used. In some embodiments, a checkerboard assay is used to determine the effect of a Chp peptide or active fragment thereof on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, PA, which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of Chp peptide or active fragment thereof diluted e.g., 2-fold along the vertical axis. The Chp peptide or active fragment thereof and antibiotic dilutions are then combined, so that each column has a constant amount of antibiotic and doubling dilutions of Chp peptide, while each row has a constant amount of Chp peptide and doubling dilutions of antibiotic. Each well thus has a unique combination of Chp peptide and antibiotic. Bacteria are added to the drug combinations at concentrations of 1×10⁵ GFU/ml in CAA, for example, with or without human serum or pulmonary surfactant. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the Chp peptide/antibiotic combination.

In some embodiments, the Chp peptides disclosed herein or active fragments thereof show low toxicity against erythrocytes. Any methodology known in the art may be used to assess the potential for hemolytic activity of the present Chp peptides or active fragments thereof.

The Chp peptides disclosed herein includes those disclosed, for example, in PCT Publication No. WO 2021/007107, incorporate by reference herein in its entirety and set forth below in Tables 1 and 2.

TABLE 1
Chlamydia phage (Chp)-derived lytic agents and Additional Chp family members

| Protein | Identifier | Protein | pI/kDa | |
|---|---|---|---|---|
| name | Information | Sequence | (amino acids) | DNA Sequence |
| Chp1 | Phage Chp1 | MVRRRRLRR | 13.23/4669.64 | ATGGTTCGTAGAAGAC |
| | Gene: Chp1p08 | RISRRIFRRTV | (36) | GTTTGAGAAGAAGAA |
| | GenBank: | ARVGRRRRS | | TAAGTAGAAGAATTTT |
| | NP_044319.1 | FRGGIRF | | TAGAAGAACAGTAGC |
| | Family: | (SEQ ID NO: 1) | | TAGAGTTGGTAGAAG |
| | Microviridae | | | GCGAAGGTCTTTTCGT |
| | | | | GGTGGTATTAGATTTT |
| | | | | AA (SEQ ID NO: 27) |
| Chp2 | Phage 2 | MRLKMARRR | 12.90/5708.98 | ATGAGGTTAAAAATG |
| | Gene: Ch-2p5 | YRLPRRRSRR | (44) | GCACGAAGAAGATAC |
| | GenBank: | LFSRTALRM | | AGACTTCCGCGACGTA |
| | NP_054652.1 | HPRNRLRRIM | | GAAGTCGAAGACTTTT |
| | Family: | RGGIRF (SEQ | | TTCAAGAACTGCATTG |
| | Microviridae | ID NO: 2) | | AGGATGCATCCAAGA |
| | | | | AATAGGCTTCGAAGA |
| | | | | ATTATGCGTGGCGGCA |
| | | | | TTAGGTTCTAG (SEQ |
| | | | | ID NO: 28) |
| CPAR39 | Phage CPAR39 | MCKKVCKKC | 10.26/3993.91 | TTGTGCAAAAAAGTGT |
| | Gene: | PKKGPKNAP | (35) | GCAAAAAATGCCCAA |
| | CPA000S | KIGAFYERKT | | AAAAAGGGCCAAAAA |
| | GenBank: | PRLKQST | | ATGCCCCCAAAATCGG |
| | NP_063898.1 | (SEQ ID NO: 3) | | AGCATTTTACGAGAGA |
| | Family: | | | AAAACACCTAGACTTA |
| | Microviridae | | | AACAGTCTACTTGA |
| | | | | (SEQ ID NO: 29) |
| Chp3 | Phage 3 | MRLKMARRR | 12.90/5708.98 | ATGAGGTTAAAAATG |
| | Gene: CP3p6 | YRLPRRRSRR | (44) | GCACGAAGAAGATAC |
| | GenBank: | LFSRTALRM | | AGACTTCCGCGACGTA |
| | YP_022484.1 | HPRNRLRRIM | | GAAGTCGAAGACTTTT |
| | Family: | RGGIRF (same | | TTCAAGAACTGCATTA |
| | Microviridae | sequence as | | AGGATGCATCCAAGA |
| | | Chp2) (SEQ ID | | AATAGGCTTCGAAGA |
| | | NO: 54) | | ATTATGCGTGGCGGCA |
| | | | | TTAGGTTCTAG (SEQ |
| | | | | ID NO: 53) |
| Chp4 | Phage 4 | MARRYRLSR | 12.88/5073.11 | ATGGCACGAAGATAC |
| | Gene: Chp4p6 | RRSRRLFSRT | (39) | AGACTTTCGCGACGCA |
| | GenBank: | ALRMHRRNR | | GAAGTCGACGACTTTT |
| | YP_338243.1 | LRRIMRGGIR | | TTCAAGAACTGCATTA |
| | Family: | F (SEQ ID NO: | | AGAATGCATCGAAGA |
| | Microviridae | 4) | | AATAGACTTCGAAGA |
| | | | | ATTATGCGTGGCGGCA |
| | | | | TTAGGTTTTAG (SEQ ID |
| | | | | NO: 30) |
| Chp5 | Phage ChpQE | MAEQYELSQ | 3.73/4605.01 | ATGGCGGAACAGTAT |
| | Derivative of | EQSEQLFSET | (39) | GAACTGAGCCAGGAA |
| | Phage 4 | ALQMHEQNE | | CAGAGCGAACAGCTG |
| | | LQEIMQGGIE | | TTTAGCGAAACCGCGC |
| | | F (SEQ ID NO: | | TGCAGATGCATGAACA |
| | | 5) | | GAACGAACTGCAGGA |
| | | | | AATTATGCAGGGCGGC |
| | | | | ATTGAATTTTAA (SEQ |
| | | | | ID NO: 31) |
| Chp6 | Guinea pig | MARRRYRLP | 12.88/5180.27 | ATGGCACGAAGAAGA |
| | Chlamydia | RRRSRRLFSR | (40) | TACAGACTTCCGCGAC |
| | phage | TALRMHPRN | | GTAGAAGTCGAAGAC |
| | GenBank: | RLRRIMRGGI | | TTTTTTCAAGAACTGC |
| | NP_510878.1 | RF (SEQ ID | | ATTAAGGATGCATCCA |
| | Family: | NO: 6) | | AGAAATAGGCTTCGA |
| | Microviridae | | | AGAATTATGCGTGGCG |
| | | | | GCATTAGGTTCTAG |
| | | | | (SEQ ID NO: 32) |
| Chp7 | Uncharacterized | MKRRKMTRK | 12.31/4302.19 | ATGAAACGTAGAAAA |
| | protein | GSKRLFTATA | (38) | ATGACAAGAAAAGGT |
| | [Chlamydia | DKTKSINTAP | | TCTAAGCGTCTTTTTA |
| | trachomatis] | PPMRGGIRL | | CTGCAACTGCTGATAA |
| | GenBank: | (SEQ ID NO: 7) | | AACTAAATCTATCAAT |
| | CRH73061.1 | | | ACTGCCCCGCCGCCAA |
| | Family: | | | TGCGTGGCGGTATCCG |
| | Microviridae | | | GTTGTAA (SEQ ID NO: |
| | | | | 33) |
| Chp8 | Uncharacterized | MSKKRSRMS | 12.91/4672.61 | ATGTCTAAAAAGCGTT |
| | protein (C. | RRRSKKLFSK | (39) | CTCGCATGTCTCGCCG |
| | trachomatis) | TALRTKSVNT | | CCGTTCTAAGAAGTTG |
| | GenBank: | RPPMRGGFRF | | TTCTCGAAAACGGCTC |
| | CRH64983.1 | (SEQ ID NO: 8) | | TCCGCACGAAGAGTGT |
| | Family: | | | CAACACCCGTCCGCCT |
| | Microviridae | | | ATGCGCGGAGGGTTCC |
| | | | | GGTTCTGA (SEQ ID |
| | | | | NO: 34) |
| Chp9 | Uncharacterized | MSLRRHKLS | 12.91/4672.60 | ATGTCTCTTCGTCGTC |
| | protein (C. | RKASKRIFRK | (40) | ATAAGCTTTCTCGTAA |
| | trachomatis) | GASRTKTLNT | | GGCGTCTAAGCGTATT |
| | GenBank: | RATPMRGGF | | TTTCGTAAAGGTGCAT |
| | CRH84960.1 | RI (SEQ ID | | CACGCACGAAGACTTT |
| | Family: | NO: 9) | | GAATACTCGTGCTACG |
| | Microviridae | | | CCTATGCGCGGCGGTT |
| | | | | TCCGTATTTAA (SEQ ID |
| | | | | NO: 35) |
| Chp10 | Uncharacterized | MKRRKLSKK | 12.91/4570.64 | ATGAAACGTCGTAAAC |
| | protein (C. | KSRKIFTRGA | (38) | TGTCCAAAAAGAAATC |
| | trachomatis) | VNVKKRNLR | | TCGCAAGATTTTCACT |
| | GenBank: | ARPMRGGFRI | | CGCGGTGCTGTAAATG |
| | CRH93270.1 | (SEQ ID NO: | | TGAAAAAGCGTAACCT |
| | Family: | 10) | | TCGCGCTCGCCCAATG |
| | Microviridae | | | CGCGGCGGTTTCCGGA |
| | | | | TCTAA |
| | | | | (SEQ ID NO: 36) |
| Chp11 | Uncharacterized | MAKKMTKG | 11.74/4375.32 | ATGGCTAAAAAAATG |
| | protein (C. | KDRQVFRKT | (37) | ACTAAAGGCAAGGAT |
| | trachomatis) | ADRTKKLNV | | CGTCAGGTTTTTCGTA |
| | GenBank: | RPLLYRGGIR | | AAACCGCTGATCGTAC |
| | CRH59954.1 | L (SEQ ID NO: | | TAAGAAACTCAATGTT |
| | Family: | 11) | | AGACCGTTGTTATATC |
| | Microviridae | | | GAGGAGGTATCAGATT |
| | | | | ATGA (SEQ ID NO: 37) |
| Chp12 | Uncharacterized | MAGKKMVS | 11.74/4549.53 | ATGGCAGGAAAAAAA |
| | protein (C. | KGKDRQIFRK | (39) | ATGGTATCAAAAGGA |
| | trachomatis) | TADRTKKMN | | AAAGATAGACAGATTT |
| | GenBank: | VRPLLYRGGI | | TCCGAAAAACTGCTGA |
| | CRH59965.1 | RL (SEQ ID | | TCGCACTAAAAAAATG |
| | Family: | NO: 12) | | AATGTGCGCCCGCTAT |
| | Microviridae | | | TATATCGTGGAGGTAT |
| | | | | TAGATTATGA (SEQ ID |
| | | | | NO: 38) |
| Gkh1 | Marine | MRRPRKMNY | 12.66/4974.97 | ATGAGAAGACCAAGA |
| | gokushovirus | KKSKRMFSR | (41) | AAAATGAACTATAAA |
| | Gene: | TAARTHRKN | | AAATCAAAAAGAATG |
| | V508_gp1 | SLRGSRPMR | | TTTTCACGCACAGCAG |
| | GenBank: | GGIRL (SEQ | | CGAGAACACACAGAA |
| | YP_008798245.1 | ID NO: 13) | | AAAACTCTCTAAGAGG |
| | Unclassified | | | TAGCCGACCTATGAGA |
| | Gokushovirinae | | | GGCGGAATACGTCTTT |
| | | | | AA (SEQ ID NO: 39) |
| Gkh2 | Gokushovirinae | MSKKASRKS | 12.49/3794.63 | ATGTCGAAGAAGGCG |
| | Fen672_31 | FTKGAVKVH | (34) | TCGAGGAAGAGTTTTA |
| | Gene: | KKNVPTR VP | | CTAAGGGTGCCGTTAA |
| | AFL78_gp4 | MRGGIRL | | GGTTCATAAGAAAAAT |
| | GenBank: | (SEQ ID NO: | | GTTCCTACTCGTGTTC |
| | YP_009160382.1 | 14) | | CTATGCGTGGCGGTAT |
| | Unclassified | | | TAGGCTTTAG (SEQ ID |
| | Gokushovirinae | | | NO: 40) |
| Unp1 | Unnamed | MKMRKRTD | 12.31/4104.04 | ATGAAAATGCGTAAG |
| | protein product | KRVFTRTAA | (35) | CGGACGGACAAGCGA |
| | (uncultured | KSKKVNIAPK | | GTGTTTACCCGCACCG |
| | bacterium) | IFRGGIRL | | CTGCTAAGTCCAAGAA |
| | GenBank: | (SEQ ID NO: | | AGTGAACATTGCCCCG |
| | CDL66944.1 | 15) | | AAAATTTTTAGAGGAG |
| | Circular | | | GTATCCGTCTGTGA |
| | plasmid, rat | | | (SEQ ID NO: 41) |
| | cecum | | | |
| Ecp1 | Nonstructural | MARSRRRMS | 12.70/4812.72 | ATGGCTCGTTCTCGCC |
| | protein | KRSSRRSFRK | (39) | GTCGTATGTCCAAGCG |
| | (Escherichia | YAKTHKRNF | | TTCTTCCCGTCGTTCGT |
| | coli) | KARSMRGGI | | TCCGTAAGTACGCAAA |
| | GenBank: | RL (SEQ ID | | GACGCATAAACGTAA |
| | WP_100756432.1 | NO: 16) | | CTTTAAAGCCCGCTCT |
| | SEPEC Feces | | | ATGCGTGGTGGAATTC |
| | strain | | | GTCTTTGA (SEQ ID NO: |
| | | | | 42) |
| Tma1 | Hypothetical | MESPNSRSQL | 7.80/5433.39 | ATGGAAAGCCCGAAC |
| | protein (T. | GITLYLLSTIF | (47) | AGCCGCAGCCAGCTG |
| | maritimus) | PDACFRYRRE | | GGCATTACCCTGTATC |
| | SAMN04488044_ | LPYPLVIWGV | | TGCTGAGCACCATTTT |
| | 0855 | ATLCLQ (SEQ | | TCCGGATGCGTGCTTT |
| | GenBank: | ID NO: 17) | | CGCTATCGCCGCGAAC |
| | SHG47122.1 | | | TGCCGTATCCGCTGGT |
| | | | | GATTTGGGGCGTGGCG |
| | | | | ACCCTGTGCCTGCAGT |
| | | | | AA (SEQ ID NO: 43) |
| Ecp2 | Hypothetical | MARSRRRMS | 12.66/4770.68 | ATGGCTCGTTCCCGTA |
| | protein | KRSSRRSFRK | (39) | GACGTATGTCTAAGCG |
| | EC13107_44c00010 | YAKSHKKNF | | TTCTTCCCGCCGTTCG |
| | (E. coli) | KARSMRGGI | | TTCCGCAAGTATGCGA |
| | GenBank: | RL (SEQ ID | | AGTCGCATAAGAAGA |
| | OAC14041.1 | NO: 18) | | ACTTTAAAGCCCGCTC |
| | Udder, acute | | | AATGCGTGGCGGTATC |
| | mastitis | | | CGTTTATAA (SEQ ID |
| | | | | NO: 44) |
| Osp1 | Hypothetical | MRKRMSKRV | 11.90/4389.35 | ATGAGAAAGCGAATG |
| | protein | DKKVFRRTA | (37) | TCTAAGCGTGTTGACA |
| | SAMN05216343_ | ASAKKINIDP | | AGAAGGTGTTCCGTCG |
| | 103150 | KIYRGGIRL | | TACTGCCGCATCTGCC |
| | (Oscillibacter | (SEQ ID NO: | | AAGAAGATTAACATTG |
| | sp. PC13) | 19) | | ACCCCAAGATTTACCG |
| | GenBank: | | | TGGAGGTATTCGCCTA |
| | SFP13761.1 | | | TGA (SEQ ID NO: 45) |
| Unp2 | Unnamed | MRRRRLSRR | 13.18/4757.77 | ATGAGACGTCGTCGTC |
| | protein product | TSRRFFRKGL | (37) | TATCCCGCAGAACTTC |
| | GenBank: | KVRRRNLRA | | CCGCCGTTTTTTCCGT |
| | CDL65918.1 | RPMRGGFRI | | AAAGGACTTAAGGTTC |
| | Extrachromosomal | (SEQ ID NO: | | GCCGTCGTAACCTCCG |
| | DNA | 20) | | CGCGAGACCCATGAG |
| | RGI00327 | | | AGGCGGATTCAGAATT |
| | | | | TGA (SEQ ID NO: 46) |
| Unp3 | Unnamed | MARRKKMK | 12.32/4545.51 | ATGGCACGACGCAAG |
| | protein product | GKRDKRVFK | (39) | AAGATGAAAGGCAAG |
| | GenBank: | QTANKTKAI | | CGGGATAAACGGGTG |
| | CDL65808.1 | NISPKNMRG | | TTTAAGCAGACAGCCA |
| | Extrachromosomal | GTRL (SEQ ID | | ACAAAACCAAGGCTA |
| | DNA | NO: 21) | | TCAACATCAGCCCAAA |
| | RGI00234 | | | AAACATGAGAGGGGG |
| | | | | TACGAGACTGTGA |
| | | | | (SEQ ID NO: 47) |
| Gkh3 | Hypothetical | MLTVWSDTP | 11.2/6440.82 | ATGTTAACTGTGTGGA |
| | protein (Marine | TIKRRKDMY | (53) | GTGACACCCCTACCAT |
| | gokushovirus) | RKRMSRKKS | | AAAAAGGAGAAAAGA |
| | GenBank: | KKVFAKTAM | | CATGTATAGAAAGAG |
| | AGT39941.1 | KVNKRNHVK | | AATGTCAAGAAAGAA |
| | | PMRGGYRI | | AAGTAAAAAGGTTTTT |
| | | (SEQ ID NO: | | GCAAAAACCGCAATG |
| | | 22) | | AAAGTAAATAAAAGA |
| | | | | AACCACGTTAAACCTA |
| | | | | TGCGTGGTGGATATAG |
| | | | | AATATAA (SEQ ID NO: |
| | | | | 48) |
| Unp5 | Hypothetical | MMKYRKKM | 12.04/4536.61 | ATGATGAAGTACAGA |
| | protein (Marine | SAKSSRKQFT | (39) | AAAAAAATGAGCGCT |
| | gokushovirus) | KGAMKVKG | | AAAAGTAGCCGAAAG |
| | GenBank: | KNFTKPMRG | | CAATTTACAAAAGGCG |
| | AGT39924.1 | GIRL (SEQ ID | | CCATGAAAGTGAAGG |
| | | NO: 23) | | GTAAAAACTTCACAAA |
| | | | | ACCAATGCGCGGAGG |
| | | | | CATCCGTCTATAG |
| | | | | (SEQ ID NO: 49) |
| Unp6 | Hypothetical | MRRYNVNKG | 12.31/4492.34 | ATGCGACGTTACAATG |
| | protein (Marine | KSAKKFRKQ | (38) | TAAATAAAGGTAAATC |
| | gokushovirus) | VSKTKVANL | | TGCTAAGAAGTTTCGA |
| | GenBank: | RSNPMRGGW | | AAGCAGGTAAGTAAG |
| | AGT39915.1 | RL (SEQ ID | | ACGAAGGTTGCAAAC |
| | | NO: 24) | | CTACGTTCTAATCCAA |
| | | | | TGCGAGGTGGTTGGAG |
| | | | | ACTCTAA (SEQ ID NO: |
| | | | | 50) |
| Spi1 | Hypothetical | MAYRGFKTS | 12.37/3776.45 | ATGGCTTATCGTGGTT |
| | protein Sp-4p3 | RVVKHRVRR | (28) | TTAAAACGAGTCGTGT |
| | (Spiroplasma | RWFNHRRRY | | TGTAAAACATAGAGTA |
| | virus SpV4] | R (SEQ ID NO: | | CGTAGAAGATGGTTTA |
| | Orf9 | 25) | | ATCATAGAAGACGTTA |
| | NCBI Ref. Seq: | | | TAGATAG (SEQ ID NO: |
| | NP_598337.1 | | | 51) |
| Spi2 | Hypothetical | MRRKVKNTK | 12.91/4629.45 | ATGCGTCGTAAAGTTA |
| | protein Sp-4p2 | RHQWRLTHS | (38) | AAAACACCAAACGTC |
| | (Spiroplasma | ARSIKRANIM | | ACCAGTGGCGTCTGAC |
| | virus SpV4) | PSNPRGGRRF | | CCACTCTGCTCGTTCT |
| | Orf8 | (SEQ ID: 26) | | ATCAAACGTGCTAACA |
| | NCBI Ref. Seq: | | | TCATGCCGTCTAACCC |
| | NP_598336.1 | | | GCGTGGTGGTCGTCGT |
| | | | | TTC (SEQ ID NO: 52) |
| Ecp3 | Nonstructural | MARSRRRMS | 12.76/4784.69 | ATGGCTCGTTCTCGTC |
| | protein | KRSSRRSFRK | (39) | GTCGTATGTCTAAACG |
| | (Escherichia) | YAKTHKKNF | | TTCTTCTCGTCGTTCTT |
| | NCBI Ref. Seq: | KARSMRGGI | | TTCGTAAATATGCTAA |
| | WP_105269219.1 | RL (SEQ ID | | AACTCATAAAAAAAA |
| | | NO: 55) | | TTTTAAAGCTCGTTCT |
| | | | | ATGCGTGGAGGAATTC |
| | | | | GTTTATAA (SEQ ID |
| | | | | NO: 68) |
| Ecp4 | Nonstructural | MARSRRRMS | 12.66/4770.68 | ATGGCGCGCAGCCGCC |
| | protein | KRSSRRSFRK | (39) | GCCGCATGAGCAAAC |
| | (Escherichia) | YAKSHKKNF | | GCAGCAGCCGCCGCA |
| | NCBI Ref. Seq: | KARSMRGGI | | GCTTTCGCAAATAT |
| | WP_105466506.1 | RL (SEQ ID | | GCGAAAAGCCATAAA |
| | | NO: 56) | | AAAAACTTTAAAGCGC |
| | | | | GCAGCATGCGCGGCG |
| | | | | GCATTCGCCTG (SEQ |
| | | | | ID NO: 69) |
| Lvp1 | Lysis protein | MSSTLCRWA | 9.7/6346.6 | ATGTCTTCTACCCTGT |
| | (Pseudomonas | VKALRCTRV | (55) | GCCGTTGGGCTGTTAA |
| | phage PP7) | YKEFIWKPLV | | AGCTCTGCGTTGCACC |
| | NCBI Ref. Seq: | ALSYVTLYLL | | CGTGTTTACAAAGAAT |
| | NP_042306.1 | SSVFLSQLSY | | TCATCTGGAAACCGCT |
| | | PIGSWAV | | GGTTGCTCTGTCTTAC |
| | | (SEQ ID NO: | | GTTACCCTGTACCTGC |
| | | 57) | | TGTCTTCTGTTTTCCTG |
| | | | | TCTCAGCTGTCTTACC |
| | | | | CGATCGGTTCTTGGGC |
| | | | | TGTT (SEQ ID NO: 70) |
| (ABP1) | Lysis protein | MKKRTKALL | 9.93/4247.21 | ATGAAGAAAAGGACA |
| Lvp2 | (Acinetobacter | PYAVFIILSFQ | (35) | AAAGCCTTGCTTCCCT |
| | phage AP205) | LTLLTALFMY | | ATGCGGTTTTCATCAT |
| | NCBI Ref. Seq: | YHYTF (SEQ | | ACTCAGCTTTCAACTA |
| | NP_085469.1 | ID NO: 58) | | ACATTGTTGACTGCCT |
| | | | | TGTTTATGTATTACCA |
| | | | | TTATACCTTTTAG (SEQ |
| | | | | ID NO: 71) |
| ALCES1 | Hypothetical | MAKKIRNKA | 12.70/4599.52 | ATGGCAAAGAAAATT |
| | protein (Alces | RDRRIFTRTA | (38) | AGAAACAAAGCACGT |
| | alces faeces | SRMHKANRT | | GATAGACGTATCTTCA |
| | associated | PRFMRGGIRL | | CAAGAACAGCTTCACG |
| | microvirus | (SEQ ID NO: | | CATGCACAAGGCAAA |
| | MP12 5423) | 59) | | CCGCACACCAAGATTT |
| | NCBI Ref. Seq: | | | ATGAGAGGCGGTATTA |
| | AXB22573.1 | | | GGTTATGA (SEQ ID |
| | | | | NO: 72) |
| AVQ206 | Hypothetical | MRRKKMSRG | 13.10/4680.78 | ATGCGTCGTAAAAAA |
| | protein | KSKKLFRRTA | (38) | ATGTCTCGTGGTAAAT |
| | (Gokushovirinae | KRVHRKNLR | | CTAAAAAACTGTTCCG |
| | environmental | ARPMRGGIR | | TCGTACCGCTAAACGT |
| | samples) | M (SEQ ID | | GTTCACCGTAAAAACC |
| | NCBI Ref. Seq: | NO: 60) | | TGCGTGCTCGTCCGAT |
| | AVQ10236.1 | | | GCGTGGTGGTATCCGT |
| | | | | ATG (SEQ ID NO: 73) |
| AVQ244 | Hypothetical | MAKRHKIPQ | 12.8/4566.43 | ATGGCTAAACGTCACA |
| | protein | RASQHSFTRH | (39) | AAATCCCGCAGCGTGC |
| | (Gokushovirinae | AQKVHPKNV | | TTCTCAGCACTCTTTC |
| | environmental | PRLPMRGGIR | | ACCCGTCACGCTCAGA |
| | samples) | L (SEQ ID NO: | | AAGTTCACCCGAAAA |
| | NCBI Ref. Seq: | 61) | | ACGTTCCGCGTCTGCC |
| | AVQ10244.1 | | | GATGCGTGGTGGTATC |
| | | | | CGTCTG (SEQ ID NO: |
| | | | | 74) |
| CDL907 | Unnamed | MRKKMHKSL | 11.96/4398.22 | ATGCGTAAAAAAATG |
| | protein product | DKRVFNRTA | (37) | CACAAATCTCTGGACA |
| | (uncultured | KKSKKINVNP | | AACGTGTTTTCAACCG |
| | bacterium) | VVYRGGIRL | | TACCGCTAAAAAATCT |
| | NCBI Ref. Seq: | (SEQ ID NO: | | AAAAAAATCAACGTT |
| | CDL65907.1 | 62) | | AACCCGGTTGTTTACC |
| | | | | GTGGTGGTATCCGTCT |
| | | | | G (SEQ ID NO: 75) |
| AGT915 | Hypothetical | MRRYNVNKG | 12.41/4492.32 | ATGCGACGTTACAATG |
| | protein (Marine | KSAKKFRKQ | (38) | TAAATAAAGGTAAATC |
| | gokushovirus) | VSKTKVANL | | TGCTAAGAAGTTTCGA |
| | NCBI Ref. Seq: | RSNPMRGGW | | AAGCAGGTAAGTAAG |
| | AGT39915.1 | RL (SEQ ID | | ACGAAGGTTGCAAAC |
| | | NO: 63) | | CTACGTTCTAATCCAA |
| | | | | TGCGAGGTGGTTGGAG |
| | | | | ACTCTAA (SEQ ID NO: |
| | | | | 76) |
| HH3930 | Hypothetical | MRPVKRSRV | 12.95/4755.69 | ATGCGTCCAGTTAAAA |
| | protein | NKARSAGKF | (41) | GATCAAGAGTAAATA |
| | RINTHH_3930 | RKQVGKTKM | | AGGCCCGATCTGCAGG |
| | (Richelia | ANLRSNPMR | | CAAGTTTCGTAAGCAG |
| | intracellularis | GGWRL (SEQ | | GTCGGTAAAACAAAG |
| | HH01) | ID NO: 64) | | ATGGCAAATCTGCGTA |
| | NCBI Ref. Seq: | | | GTAATCCGATGCGCGG |
| | CCH66548.1 | | | CGGATGGCGGCTGTGA |
| | | | | (SEQ ID NO: 77) |
| Fen7875 | Hypothetical | MKPLKRKPV | 12.81/4699.7 | ATGAAGCCATTGAAGC |
| | protein | QKARSAAKF | (41) | GTAAGCCGGTTCAGAA |
| | (Gokushovirinae | RRNVSTVKA | | GGCGCGGTCAGCAGC |
| | Fen7875_21) | ANMAVKPM | | CAAGTTCCGTCGAAAT |
| | NCBI Ref. Seq: | RGGWRF | | GTGTCTACCGTTAAGG |
| | YP_009160399.1 | (SEQ ID NO: | | CTGCCAATATGGCGGT |
| | | 65) | | GAAGCCGATGCGCGG |
| | | | | CGGTTGGCGGTTCTGA |
| | | | | (SEQ ID NO: 78) |
| SBR77 | Hypothetical | MTKRDIEYR | 11.48/4882.78 | ATGACCAAGAGAGAC |
| | protein | KALGLNPSEP | (44) | ATCGAGTACCGGAAA |
| | SEA_BABYRAY_ | LPKIVGAVTR | | GCTTTGGGGCTCAACC |
| | 77 | HGATLKRPR | | CATCTGAGCCGCTCCC |
| | (Mycobacterium | VTALAR (SEQ | | GAAGATTGTGGGTGCC |
| | phage | ID NO: 66) | | GTCACCCGCCACGGGG |
| | BabyRay) | | | CCACTCTGAAACGCCC |
| | NCBI Ref. Seq: | | | ACGGGTCACCGCACTG |
| | AOT25441 | | | GCCCGATAG (SEQ ID |
| | | | | NO: 79) |

| Protein | Identifier | Protein | pI/kDa | |
|---|---|---|---|---|
| name | Information | Sequence | (amino acids) | DNA Sequence |
| Bdp1 | Putative DNA | MKRKPMSRK | 12.9/5708.98 | ATGAAAAGAAAACCA |
| | binding protein | ASQKTFKKN | (38) | ATGAGCCGCAAGGCCT |
| | (Bdellovibrio | TGVQRMNHL | | CTCAAAAAACCTTCAA |
| | phage | NPRAMRGGI | | AAAGAACACAGGCGT |
| | phiMH2K) | RL (SEQ ID | | TCAACGCATGAACCAT |
| | NCBI Ref. Seq: | NO: 67) | | CTCAACCCACGCGCCA |
| | NP_073546.1 | | | TGCGTGGTGGCATTAG |
| | | | | ACTATAA (SEQ ID NO: |
| | | | | 80) |
| Unp4 | Hypothetical | MIVRRHKMS | 12.8/4918.88 | ATGATCGTTCGTCGTC |
| | protein (Marine | RRRSRKLFSK | (40) | ACAAAATGTCTCGTCG |
| | gokushovirus) | TASRTRSKNL | | TCGTTCTCGTAAACTG |
| | NCBI Ref. Seq: | RSRPMRGGY | | TTCTCTAAAACCGCTT |
| | WP_113076974.1 | RI (SEQ ID | | CTCGTACCCGTTCTAA |
| | | NO: 94) | | AAACCTGCGTTCTCGT |
| | | | | CCGATGCGTGGTGGTT |
| | | | | ACCGTATC (SEQ ID |
| | | | | NO: 103) |
| Myo1 | Hypothetical | MKLTKSDIA | 11.7/5287.23 | ATGAAACTGACCAAAT |
| | protein Peaches | YREALGLSTT | (46) | CTGACATCGCTTACCG |
| | 72 | DPLPAEIGMV | | TGAAGCTCTGGGTCTG |
| | (Mycobacterium | TRRANRLKR | | TCTACCACCGACCCGC |
| | virus | PRKTARFR | | TGCCGGCTGAAATCGG |
| | Peaches) NCBI | (SEQ ID NO: | | TATGGTTACCCGTCGT |
| | Ref. Seq: | 102) | | GCTAACCGTCTGAAAC |
| | YP_003358775.1 | | | GTCCGCGTAAAACCGC |
| | | | | TCGTTTCCGT (SEQ ID |
| | | | | NO: 104) |

TABLE 2
Modified Chp peptide family members

| Protein | Identifier | Protein | pI/kDa | |
|---|---|---|---|---|
| name | Information | Sequence | (amino acids) | DNA Sequence |
| Chp2-M1 | Modified | MRLKMARRR | 12.9/5708.98 | ATGAGGTTAAAAATG |
| | variant of | YRLPRRRSRR | (44) | GCACGAAGAAGATAC |
| | Chp2 | LFSRTALRM | | AGACTTCCGCGACGTA |
| | | HPRNRLRRIM | | GAAGTCGAAGACTTTT |
| | | RGGIRF (SEQ | | TTCAAGAACTGCATTG |
| | | ID NO: 81) | | AGGATGCATCCAAGA |
| | | | | AATAGGCTTCGAAGA |
| | | | | ATTATGCGTGGCGGCA |
| | | | | TTAGGTTC (SEQ ID NO: |
| | | | | 105) |
| Chp2-Cys | Modified | MRLKMARRR | 12.8/5812.15 | ATGAGGTTAAAAATG |
| | variant of | YRLPRRRSRR | (45) | GCACGAAGAAGATAC |
| | Chp2 | LFSRTALRM | | AGACTTCCGCGACGTA |
| | | HPRNRLRRIM | | GAAGTCGAAGACTTTT |
| | | RGGIRFC | | TTCAAGAACTGCATTG |
| | | (SEQ ID NO: | | AGGATGCATCCAAGA |
| | | 82) | | AATAGGCTTCGAAGA |
| | | | | ATTATGCGTGGCGGCA |
| | | | | TTAGGTTCTGT (SEQ ID |
| | | | | NO: 106) |
| Chp2-NC | Modified | APKAMRLKM | 13.00/6744.29 | GCTCCTAAAGCTAGGT |
| | variant of | ARRRYRLPR | (54) | TAAAAATGGCACGAA |
| | Chp2 | RRSRRLFSRT | | GAAGATACAGACTTCC |
| | | ALRMHPRNR | | GCGACGTAGAAGTCG |
| | | LRRIMRGGIR | | AAGACTTTTTTCAAGA |
| | | FLQKKGI | | ACTGCATTGAGGATGC |
| | | (SEQ ID NO: | | ATCCAAGAAATAGGCT |
| | | 83) | | TCGAAGAATTATGCGT |
| | | | | GGCGGCATTAGGTTCT |
| | | | | TACAAAAAAAAGGAA |
| | | | | TT (SEQ ID NO: 107) |
| Chp4::Chp2 | Fusion | MARRYRLSR | 13.18/7851.54 | ATGGCTCGTCGTTATC |
| | peptide of | RRSRRLFSRT | (60) | GTTTATCTCGTCGTCG |
| | Chp4 and | ALRMHRRNR | | TTCTCGTCGTTTATTTT |
| | Chp2 | LRRIMRRLFS | | CTCGTACTGCTTTACG |
| | | RTALRMHRR | | TATGCATCGTCGTAAT |
| | | NRLRRIMRG | | CGTTTACGTCGTATTA |
| | | GIRF (SEQ ID | | TGCGTCGTTTATTTTCT |
| | | NO: 84) | | CGTACTGCTTTACGTA |
| | | | | TGCATCGTCGTAATCG |
| | | | | TTTACGTCGTATTATG |
| | | | | CGTGGAGGAATTCGTT |
| | | | | TT (SEQ ID NO: 108) |
| Chp2-CAV | Modified | GRLYRFHRPR | 13.00/5709 | GGACGTTTATATCGTT |
| | variant of | RRNAIGMSR | (44) | TTCATCGTCCTCGTCG |
| | Chp2 | MRRKMFLRR | | TCGTAATGCTATTGGA |
| | | MLRLISRRTR | | ATGTCTCGTATGCGTC |
| | | RPRLRA (SEQ | | GTAAAATGTTTTTACG |
| | | ID NO: 85) | | TCGTATGTTACGTTTA |
| | | | | ATTTCTCGTCGTACTC |
| | | | | GTCGTCCTCGTTTACG |
| | | | | TGCT (SEQ ID NO: 109) |
| Ecp1-CAV | Modified | RTRNFRIRRA | 12.80/4812.71 | CGTACTCGTAATTTTC |
| | variant of | KARRKMMLS | (39) | GTATTCGTCGTGCTAA |
| | Ecp1 | HFKYGMARK | | AGCTCGTCGTAAAATG |
| | | GSKSRSSRRS | | ATGTTATCTCATTTTA |
| | | R (SEQ ID NO: | | AATATGGAATGGCTCG |
| | | 86) | | TAAAGGATCTAAATCT |
| | | | | CGTTCTTCTCGTCGTTC |
| | | | | TCGT (SEQ ID NO: 110) |
| Ecp1-M1 | Modified | MARSRRRMS | 12.7/4812.72 | ATGGCTCGTTCTCGTC |
| | variant of | KRSSRRSFRK | (39) | GTCGTATGTCTAAACG |
| | Ecp1 | YAKTHKRNF | | TTCTTCTCGTCGTTCTT |
| | | KARSMRGGI | | TTCGTAAATATGCTAA |
| | | RL (SEQ ID | | AACTCATAAACGTAAT |
| | | NO: 87) | | TTTAAAGCTCGTTCTA |
| | | | | TGCGTGGAGGAATTCG |
| | | | | TTTATAA (SEQ ID NO: |
| | | | | 111) |
| Chp6-M1 | Modified | MARRRYRLP | 12.31/4492.34 | ATGGCACGAAGAAGA |
| | variant of | RRRSRRLFSR | (38) | TACAGACTTCCGCGAC |
| | Chp6 | TALRMHPRN | | GTAGAAGTCGAAGAC |
| | | RLRRIMRGGI | | TTTTTTCAAGAACTGC |
| | | RF (SEQ ID | | ATTAAGGATGCATCCA |
| | | NO: 88) | | AGAAATAGGCTTCGA |
| | | | | AGAATTATGCGTGGCG |
| | | | | GCATTAGGTTCTAG |
| | | | | (SEQ ID NO: 112) |
| Chp10-M1 | Modified | MKRRKLSKK | 12.91/4570.64 | GTGAAACGTCGTAAAC |
| | variant of | KSRKIFTRGA | (38) | TGTCCAAAAAGAAATC |
| | Chp10 | VNVKKRNLR | | TCGCAAGATTTTCACT |
| | | ARPMRGGFRI | | CGCGGTGCTGTAAATG |
| | | (SEQ ID NO: | | TGAAAAAGCGTAACCT |
| | | 89) | | TCGCGCTCGCCCAATG |
| | | | | CGCGGCGGTTTCCGGA |
| | | | | TCTAA (SEQ ID NO: |
| | | | | 113) |
| Mse-M1 | Modified | MAKKIRNKA | 12.70/4599.52 | ATGGCTAAAAAAATCC |
| | variant of | RDRRIFTRTA | (38) | GTAACAAAGCTCGTGA |
| | Mse | SRMHKANRT | | CCGTCGTATCTTCACC |
| | | PRFMRGGIRL | | CGTACCGCTTCTCGTA |
| | | (SEQ ID NO: | | TGCACAAAGCTAACCG |
| | | 90) | | TACCCCGCGTTTCATG |
| | | | | CGTGGTGGTATCCGTC |
| | | | | TG (SEQ ID NO: 114) |
| Chp4-M1 | Modified | MARRYRLSR | 12.88/5073.11 | ATGGCACGAAGATAC |
| | variant of | RRSRRLFSRT | (39) | AGACTTTCGCGACGCA |
| | Chp4 | ALRMHRRNR | | GAAGTCGACGACTTTT |
| | | LRRIMRGGIR | | TTCAAGAACTGCATTA |
| | | F (SEQ ID NO: | | AGAATGCATCGAAGA |
| | | 91) | | AATAGACTTCGAAGA |
| | | | | ATTATGCGTGGCGGCA |
| | | | | TTAGGTTTTAG (SEQ ID |
| | | | | NO: 115) |
| Chp2-SCR1 | Modified | MRLRYGHRR | 13.00/5709 | ATGCGTCTGCGTTACG |
| | variant of | MTAGRIRMR | (44) | GTCACCGTCGTATGAC |
| | Chp2 | SRRKFMLPRF | | CGCTGGTCGTATCCGT |
| | | RLLRIPRRSN | | ATGCGTTCTCGTCGTA |
| | | RRRLRA (SEQ | | AATTCATGCTGCCGCG |
| | | ID NO: 92) | | TTTCCGTCTGCTGCGT |
| | | | | ATCCCGCGTCGTTCTA |
| | | | | ACCGTCGTCGTCTGCG |
| | | | | TGCT (SEQ ID NO: 116) |
| Chp2-SCR- | Modified | MRLRYGHRR | 13.00/5709 | ATGCGTCTGCGTTACG |
| M1 | variant of | MTAGRIRMR | (44) | GTCACCGTCGTATGAC |
| | Chp2 | SRRKFMLPRF | | CGCTGGTCGTATCCGT |
| | | RLLRIPRRSN | | ATGCGTTCTCGTCGTA |
| | | RRRLRA (SEQ | | AATTCATGCTGCCGCG |
| | | ID NO: 93) | | TTTCCGTCTGCTGCGT |
| | | | | ATCCCGCGTCGTTCTA |
| | | | | ACCGTCGTCGTCTGCG |
| | | | | TGCT (SEQ ID NO: 117) |
| Chp7-M1 | Modified | MKRRKMTRK | 12.31/4302.19 | ATGAAACGTAGAAAA |
| | variant of | GSKRLFTATA | (38) | ATGACAAGAAAAGGT |
| | Chp7 | DKTKSINTAP | | TCTAAGCGTCTTTTTA |
| | | PPMRGGIRL | | CTGCAACTGCTGATAA |
| | | (SEQ ID NO: | | AACTAAATCTATCAAT |
| | | 95) | | ACTGCCCCGCCGCCAA |
| | | | | TGCGTGGCGGTATCCG |
| | | | | GTTGTAA (SEQ ID NO: |
| | | | | 118) |
| Osp1-M1 | Modified | MRKRMSKRV | 11.9/4389.35 | ATGAGAAAGCGAATG |
| | variant of | DKKVFRRTA | (37) | TCTAAGCGTGTTGACA |
| | Osp1 | ASAKKINIDP | | AGAAGGTGTTCCGTCG |
| | | KIYRGGIRL | | TACTGCCGCATCTGCC |
| | | (SEQ ID NO: | | AAGAAGATTAACATTG |
| | | 96) | | ACCCCAAGATTTACCG |
| | | | | TGGAGGTATTCGCCTA |
| | | | | TGA (SEQ ID NO: 119) |
| Unp2-M1 | Modified | MRRRRLSRR | 13.18/4757.77 | ATGAGACGTCGTCGTC |
| | variant of | TSRRFFRKGL | (37) | TATCCCGCAGAACTTC |
| | Unp2 | KVRRRNLRA | | CCGCCGTTTTTTCCGT |
| | | RPMRGGFRI | | AAAGGACTTAAGGTTC |
| | | (SEQ ID NO: | | GCCGTCGTAACCTCCG |
| | | 97) | | CGCGAGACCCATGAG |
| | | | | AGGCGGATTCAGAATT |
| | | | | TGA (SEQ ID NO: 120) |
| Unp3-M1 | Modified | MARRKKMKG | 12.32/4545.51 | ATGGCACGACGCAAG |
| | variant of | KRDKRVFKQ | (39) | AAGATGAAAGGCAAG |
| | Unp3 | TANKTKAINI | | CGGGATAAACGGGTG |
| | | SPKNMRGGT | | TTTAAGCAGACAGCCA |
| | | RL (SEQ ID | | ACAAAACCAAGGCTA |
| | | NO: 98) | | TCAACATCAGCCCAAA |
| | | | | AAACATGAGAGGGGG |
| | | | | TACGAGACTGTGA |
| | | | | (SEQ ID NO: 121) |
| Spi2-M1 | Modified | MRRKVKNTK | 12.91/4629.45 | ATGCGTCGTAAAGTTA |
| | variant of | RHQWRLTHS | (38) | AAAACACCAAACGTC |
| | Spi2 | ARSIKRANIM | | ACCAGTGGCGTCTGAC |
| | | PSNPRGGRR | | CCACTCTGCTCGTTCT |
| | | (SEQ ID NO: | | ATCAAACGTGCTAACA |
| | | 99) | | TCATGCCGTCTAACCC |
| | | | | GCGTGGTGGTCGTCGT |
| | | | | TTC (SEQ ID NO: 122) |
| Ecp3-M1 | Modified | MARSRRRMS | 12.76/4784.69 | ATGGCTCGTTCTCGTC |
| | variant of | KRSSRRSFRK | (39) | GTCGTATGTCTAAACG |
| | Ecp3 | YAKTHKKNF | | TTCTTCTCGTCGTTCTT |
| | | KARSMRGGI | | TTCGTAAATATGCTAA |
| | | RL (SEQ ID | | AACTCATAAAAAAAA |
| | | NO: 100) | | TTTTAAAGCTCGTTCT |
| | | | | ATGCGTGGAGGAATTC |
| | | | | GTTTATAA (SEQ ID |
| | | | | NO: 123) |
| Agt1-M1 | Modified | MRRYNVNKG | 12.41/5287.23 | ATGCGACGTTACAATG |
| | variant of | KSAKKFRKQ | (46) | TAAATAAAGGTAAATC |
| | Agt1 | VSKTKVANL | | TGCTAAGAAGTTTCGA |
| | | RSNPMRGGW | | AAGCAGGTAAGTAAG |
| | | RL (SEQ ID | | ACGAAGGTTGCAAAC |
| | | NO: 101) | | CTACGTTCTAATCCAA |
| | | | | TGCGAGGTGGTTGGAG |
| | | | | ACTCTAA (SEQ ID NO: |
| | | | | 124) |

### Lysins, Variant Lysins, Active Fragments Thereof or Derivatives

The present disclosure is also directed to isolated polypeptides comprising lysins, variant lysins, active fragments thereof or derivatives. In some embodiments, the isolated polypeptides comprising the lysins, variant lysins, active fragments thereof or derivatives are combined with antimicrobial peptides (“AMPs”) to form a lysin-AMP construct, such as the lysin-AMP construct of SEQ ID NO: 149 (GN370), wherein the lysin-AMP construct has lytic activity. As used herein “lytic activity” encompasses the ability of a lysin to kill bacteria (e.g., P. aeruginosa), reduce the population of bacteria or inhibit bacterial growth (e.g., by penetrating the outer membrane of a Gram-negative bacteria), optionally in the presence of human serum or pulmonary surfactant. Lytic activity also encompasses the ability to remove or reduce a biofilm and/or the ability to reduce the minimum inhibitory concentration (MIC) of an antibiotic, optionally in the presence of human serum or pulmonary surfactant. Exemplary lysins and lysin-AMP constructs are described, for instance, in WO 2019/191633 and WO 2020/046747, incorporate by reference herein in their entireties.

As with Chp peptides discussed above, the ability of a lysin to penetrate an outer membrane of a Gram-negative bacteria may be assessed by any method known in the art, such as described in WO 2017/049233, which is herein incorporated by reference in its entirety. For example, the lysin may be incubated with Gram-negative bacteria and a hydrophobic compound. Most Gram-negative bacteria are strongly resistant to hydrophobic compounds, due to the presence of the outer membrane and, thus, do not allow the uptake of hydrophobic agents such as 1-N-phenylnaphthylamine (NPN), crystal violet, or 8-anilino-1-naphthalenesulfonic acid (ANS). NPN, for example, fluoresces strongly under hydrophobic conditions and weakly under aqueous conditions. Accordingly, NPN fluorescence can be used as a measurement of the outer membrane permeability.

More particularly, the ability of a lysin to penetrate an outer wall may be assessed by incubating, e.g., NPN with a Gram-negative bacteria, e.g., P. aeruginosa strain PA01, in the presence of the lysin to be tested for activity. A higher induction of fluorescence in comparison to the fluorescence emitted in the absence of a lysin (negative control) indicates outer membrane penetration. In addition, fluorescence induction can be compared to that of established permeabilizing agents, such as EDTA (ethylene diamine tetraacetate) or an antibiotic such as an antibiotic of last resort used in the treatment of P. aeruginosa, i.e., Polymyxin B (PMB) to assess the level of outer membrane permeability.

In some embodiments, the present isolated polypeptides comprising lysins, variant lysins, active fragments thereof or derivatives exhibit lytic activity in the presence and/or absence of human serum. Suitable methods for assessing the activity of a lysin in human serum are known in the art and described in the examples. Briefly, a MIC value (i.e., the minimum concentration of peptide sufficient to suppress at least 80% of the bacterial growth compared to control) may be determined for a lysin and compared to, e.g., a parent lysin or compound inactive in human serum, e.g., T4 phage lysozyme or artilysin GN126, as disclosed in WO 2020/046747 (pI 9.8). T4 phage lysozyme is commercially available, e.g. from Sigma-Aldrich, Inc. GN126 corresponds to Art-175, which is described in the literature and is obtained by fusing AMP SMAP-29 to GN lysin KZ144. See Briers et al. 2014, Antimicrob, Agents Chemother. 58:3774-3784, which is herein incorporated by reference in its entirety. Lysin GN65 (pI9.9) and dispersin B, which is an enzyme that degrades biofilm (GN81, pI 6.0), both of which are disclosed, for example, in WO 2020/046747, may also be used as controls.

More particularly, MIC values for a lysin may be determined against e.g., the laboratory P. aeruginosa strain PA01, in e.g., Mueller-Hinton broth, Mueller-Hinton broth supplemented with human serum, CAA as described herein, which includes physiological salt concentrations, and CAA supplemented with human serum. The use of PA01 enables testing in the presence of elevated serum concentrations since unlike most clinical isolates, PA01 is insensitive to the antibacterial activity of human blood matrices.

In some embodiments, the present isolated polypeptides comprising lysins, variant lysins, active fragments thereof or derivatives are capable of reducing a biofilm. Methods for assessing the Minimal Biofilm Eradicating Concentration (MBEC) of a lysin or AMP may be determined as discussed above for Chp peptides.

In some embodiments, the present isolated polypeptides comprising lysins, variant lysins, active fragments thereof or derivatives reduce the minimum inhibitory concentration (MIC) of an antibiotic needed to inhibit bacteria in the presence and/or absence of human serum, pulmonary surfactant, or sputum, including human sputum and human sputum from a cystic fibrosis patient. Any known method to assess MIC may be used. In some embodiments, a checkerboard assay, as discussed above, is used to determine the effect of a lysin on antibiotic concentration.

In some embodiments, the present lysins and lysin-AMP constructs are able to synergize with antibiotics, such as amikacin, imipenem and meropenem, and drive the resensitization of Gram-negative bacteria including MDR and/or XDR organisms, such as carbapenem-resistant P. aeruginosa. Such resensitization may be determined by combining the present lysins or lysin-AMP constructs with an antibiotic in a checkerboard assay as described herein. Antibiotic-resistant bacteria, such as carbapenem-resistant P. aeruginosa, are added to the lysin or lysin-AMP polypeptide construct combination. Generally resensitization occurs in synergistic combinations in which the antibiotic MIC values fall below established breakpoints, e.g., a MIC value of ≤2 for antibiotic sensitive bacteria, a MIC value of 4 for intermediately sensitive bacteria and a MIC value of ≥8 for antibiotic resistant bacteria, e.g. carbapenem-resistant isolates. See Clinical and Laboratory Standards Institute (CLSI), CLSI. 2019. M100 Performance Standards for Antimicrobial Susceptibility Testing; 29th Edition. Clinical and Laboratory Standards Institute, Wayne, PA, which is herein incorporated by reference in its entirety.

In some embodiments, the present isolated polypeptides comprising lysins, variant lysins, active fragments thereof or derivatives show low toxicity against erythrocytes. Any methodology known in the art may be used to assess the potential for hemolytic activity of the present isolated polypeptides comprising lysins, variant lysins, active fragments thereof or derivatives including the methods described in PCT Publication Nos. 2019/191633 and 2020/046747.

Examples of suitable lysins of the present disclosure, particularly for use with the lysin-AMP constructs described herein, include the GN316 lysin obtained from Klebsiella phage 0507-KN2-1 (NCBI Reference Sequence: YP_008531963.1), Lysin PaP2_gpl7 obtained from Pseudomonas phage (NCBI Reference Sequence: YP_024745.1), GN333 obtained from Delftia sp. (NCBI Reference Sequence: WP_016064791.1), GN424 obtained from Burkholderia pseudomultivorans (NCBI Reference Sequence: WP_060250996.1), GN425 lysin obtained from Pseudomonas flexibilis (NCBI Reference Sequence: WP_039605935.1), GN428 obtained from Escherichia virus CBA120 (NCBI Reference Sequence: YP_004957781.1), GN431 obtained from Dickeya phage phiD3 (NCBI Reference Sequence: AIM51349.1), GN485 obtained from Erwinia sp. Leaf5 (NCBI Reference Sequence: WP_056233282.1) and GN123 obtained from Pseudomonas phage PhiPA3 (NCBI Reference Sequence: YP_009217242.1).

The above-described lysins were identified by bioinformatics techniques and are disclosed, for example, in PCT Publication No. WO 2020/046747. Although some of the identified sequences had been annotated as putative peptidoglycan binding proteins, no function had been previously definitively attributed to polypeptides having these sequences. The inventors have surprisingly recognized that the above-identified sequences are suitable for use as antibacterial agents, in particular, against Gram-negative bacteria as described in the examples.

Additional examples of suitable lysins of the present disclosure, particularly those for use with the present lysin-AMP constructs, include the GN7 (pI 5.6), obtained from a marine metagenome, NCBI Accession Number ECF75988.1; GN11 (pI7.3), obtained from Pseudomonas putida KT2440, NCBI Accession Number NP_744418.1; GN40 (pI 5.1), obtained from Pseudomonas putida strain PA14H7, NCBI Accession Number NZ_KN639176.1; GN122 (pI 5.4), obtained from Pseudomonas putida strain PA14H7, NCBI Accession Number NZ_KN639176.1; GN328 (pI7.9), obtained from Pseudomonas protegens, NCBI Accession Number NC_021237.1; GN76, obtained from Acinetobacter phage vB_AbaP_CEB1, NCBI Reference Sequence ALC76575.1, GenBank: ALC76575.1; GN4, obtained from Pseudomonas phage PAJU2, NCBI Reference Sequence YP 002284361.1; GN14, obtained from Pseudomonas phage Lull, NCBI Reference Sequence YP 006382555.1; and GN37, obtained from Micavibrio aeruginosavorus, NCBI Reference Sequence WP_014102102.1, all of which are disclosed in PCT Publication No. WO 2020/046747, which is herein incorporated by reference in its entirety. GN4, GN14 and GN37 are also disclosed in WO 2017/049233, which is herein incorporated by reference in its entirety.

Suitable lysin-AMP constructs of the present disclosure include GN75 (pI 10.1) and GN83 (pI 9.4). GN75 comprises the AMP OBPgpLYS (SEQ ID NO: 88 of U.S. Pat. No. 8,846,865) fused to the N-terminus of lysin GN13 described in WO 2019/118632. GN83 comprises the AMP OBPgpLYS (SEQ ID NO: 88 of U.S. Pat. No. 8,846,865) fused to the N-terminus of lysin GN4 described in WO 2019/118632. U.S. Pat. No. 8,846,865 and WO 2019/118632 are each incorporated herein by reference in its entirety.

In some embodiments, a suitable polypeptide of the disclosure is a dispersin B-like molecule, such as an enzyme, which is capable of disrupting biofilm formation. Suitable dispersin B-like molecules include GN80 (pI 4.6), as disclosed in PCT Publication No. WO 2020/046747.

In some embodiments, the present isolated polypeptides comprise a lysin variant, e.g., a lysin containing one or more insertions, deletions and/or amino acid substitutions in comparison to a reference lysin polypeptide, e.g., a naturally occurring lysin or a parent lysin, which itself is a variant lysin. In some embodiments, an isolated polypeptide sequence comprising a variant lysin, active fragment thereof or derivative has at least 80%, such as at least 85%, such as at least 90%, such as at least 95%, such as at least 98% or such as at least 99% sequence identity with the reference lysin (e.g., GN202) and/or active fragment thereof described herein.

The lysin variants of the present disclosure typically retain one or more functional or biological activities of a reference lysin. In some embodiments, the modification improves the antibacterial activity of the lysin. Typically, the lysin variant has improved in vitro antibacterial activity (e.g., in buffer and/or media) in comparison to the reference lysin. In other embodiments, the lysin variant has improved in vivo antibacterial activity (e.g., in an animal infection model). In some embodiments, the modification improves the antibacterial activity of the lysin in the absence and/or presence of human serum. In some embodiments, the modification improves the antibacterial activity of the lysin in the presence of pulmonary surfactant.

In some embodiments, the variant lysins are obtained by modifying a reference lysin to include a modification resulting in a change in the overall isoelectric point (pI) of the lysin, i.e., the pH at which a molecule has a net neutral charge by, for example, incorporating a single pI-increasing mutation, such as a single point mutation, into a reference lysin.

In some embodiments, the lysin variants of the present disclosure are typically designed to retain an α-helix domain, the presence or absence of which can be readily determined using various software programs, such as Jpred4 (compio.dundee.ac.uk/jpred), Helical Wheel (hael.net/helical.htm), HeliQuest (zhanglab.ccmb.med.umich.edu/I-TASSER/) and PEP-FOLD 3 (bioserv.rpbs.univ-paris-diderot.fr/services/PEP-FOLD3).

In some embodiments, the α-helix domain is located at the C terminus of a lysin. In other embodiments, the α-helix domain is located at the N-terminus of a lysin. More typically, the α-helix domain is located at the C terminus. The α-helix domain of the lysins of the present disclosure varies in size between about 20 and 40 amino acids, more typically between about 15 and 33 amino acid residues. For example, the GN14 α-helix domain, which is located at the N terminus, contains 15 amino acids (residues 66 to 80 of SEQ ID NO: 124 of PCT Publication No. WO 2020/046747). The GN37 α-helix domain, which is located at the C terminus, contains 14 amino acids (residues 113 to 126 of SEQ ID NO: 84 of PCT Publication No. WO 2020/046747). The GN4 α-helix domain, which is also located at the C terminus, contains 25 amino acids (residues 116 to 140 of SEQ ID NO: 74 of PCT Publication No. WO 2020/046747).

In some embodiments, the variant lysins, active fragments thereof or derivatives thereof disclosed herein are modified to include a purification tag, e.g. those disclosed in PCT Publication Nos. WO 2019/191633 and WO 2020/046747, both of which are incorporated by reference herein.

Lysin variants may be formed by any method known in the art and as described in WO 2017/049233, which is herein incorporated by reference in its entirety, e.g., by modifying any of the lysins, active fragments thereof and derivatives described herein through site-directed mutagenesis or via mutations in hosts that produce the present lysins which retain one or more of the biological functions as described herein. The present lysin variants may be truncated, chimeric, shuffled or “natural,” and may be in combination as described, for example, in U.S. Pat. No. 5,604,109, which is incorporated herein in its entirety by reference.

For example, one of skill in the art can reasonably make and test substitutions or replacements to, e.g., the α-helix domain or regions outside of the α-helix domain. Sequence comparisons to the Genbank database can be made with e.g., a full amino acid sequence as described herein, for instance, to identify amino acids for substitution.

Mutations can be made in the amino acid sequences, or in the nucleic acid sequences encoding the polypeptides and lysins, active fragments or derivatives, such that a particular codon is changed to a codon which codes for a different amino acid, an amino acid is substituted for another amino acid, or one or more amino acids are deleted.

Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present disclosure should be considered to include sequences containing conservative changes which do not significantly alter the activity or binding characteristics of the resulting protein. Thus, one of skill in the art, based on a review of the sequence of lysins provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for anti-bacterial activity as described herein against, e.g., P. aeruginosa, and/or for having comparable activity to the lysin(s) as described and particularly provided herein. Thus, changes made to the sequence of lysin, and mutants or variants described herein can be tested using the assays and methods known in the art and described herein. One of skill in the art, on the basis of the domain structure of the lysin(s) hereof can predict one or more, one or several amino acids suitable for substitution or replacement and/or one or more amino acids which are not suitable for substitution or replacement, including reasonable conservative or non-conservative substitutions.

In some embodiments, the present isolated polypeptides comprise active fragments of lysins or derivatives. The term “active fragment” refers to a portion of a full-length lysin, which retains one or more biological activities of the reference lysin. Thus, as used herein, an active fragment of a lysin or variant lysin inhibits the growth, or reduces the population, or kills e.g., P. aeruginosa and and/or other Gram-negative bacteria as described herein in the absence or presence of, or in both the absence and presence of, human serum or in the presence of pulmonary surfactant. Suitable active fragments of lysins include, but are not limited, to those described in WO2017/049233, which is herein incorporated by reference in its entirety. The active lysin fragments typically retain an α-helix domain.

### Anti-Microbial Peptides

In some embodiments, the polypeptides of the present disclosure comprise lysin-AMP constructs. The lysin-AMP constructs comprise an isolated polypeptide comprising a lysin, variant lysin, active fragment thereof or derivative as described herein and an antimicrobial peptide or fragment thereof. The term “antimicrobial peptide” (AMP) as used herein refers to a member of a wide range of short (generally 3 to 50 amino acid residues in length) gene-encoded peptides, typically antibiotics, that can be found in virtually every organism and can include, for example, any of the Chp peptides disclosed herein or disclosed in PCT Publication No. 2021/007107, incorporated by reference herein. The term encompasses helical peptides, j-sheet peptides and those that display largely disordered random coil structures. AMPs include defensins, cathelicidins, sushi peptides, cationic peptides, polycationic peptides, amphipathic peptides, hydrophobic peptides and/or AMP-like peptides, e.g., amurin peptides as described herein. Fragments of AMPs, AMP variants and derivatives of AMPs are also encompassed by this term.

The term “AMP activity” as used herein encompasses the ability of an AMP or fragment thereof to kill bacteria, reduce the population of bacteria or inhibit bacterial growth e.g., by penetrating the outer membrane of a Gram-negative bacteria, e.g., in the presence and/or absence of human serum or pulmonary surfactant. Typically, translocation of the AMPs is driven by a primary electrostatic interaction with the lipopolysaccharide portion of the outer membrane followed by cation displacement, membrane disorganization and transient openings, and in some cases, internalization of the AMP.

AMP activity also encompasses the ability of an AMP or fragment thereof to reduce the minimum inhibitory concentration (MIC) of an antibiotic in the presence and/or absence of human serum or pulmonary surfactant. Suitable methods for assessing the ability of the present AMPs and fragments thereof to penetrate the outer membrane of Gram-negative bacteria and determining a reduction in the MIC of an antibiotic in the presence and absence of serum or pulmonary surfactant are known in the art and include those methods described above for the present lysins, derivatives and active fragments thereof.

In some embodiments, the present AMPs are variant AMPs having at least 50%, at least 60%, at least 75%, at least 80%, such as at least 85%, such as at least 90%, such as at least 95%, such as at least 98% or such as at least 99% sequence identity with any of the AMPs described herein, wherein the variant AMP thereof retains an AMP activity.

In some embodiments, the present AMPs comprise a helical domain, such as an α-helical domain. In some embodiments, the α-helical domain spans most of the molecule. See, for example, Chp1 and Chp4 of FIG. 1. In other embodiments, the α-helix domain is either interrupted (e.g., Chp2) or truncated (e.g., Chp6 and Osp1). The α-helix domain of the present AMPs, such as the Chps, described herein vary in size from between about 3 to 32 amino acids, more typically between about 10 and 25 amino acid residues. Generally, the helical domains are required for activity and typically must be retained when fused to a C- or N-terminus of a lysin.

Typically, helical peptides display amphipathic characteristics and contain a substantial proportion (e.g. 50%) of hydrophobic residues, frequently appearing in repeated patterns. Upon formation of an α-helical structure, the hydrophilic residues typically end up on the same side of the helix, thereby resulting in a conformation-dependent amphiphilicity. Frequently, these peptides are unstructured in an aqueous environment, but adopt a helical conformation upon encountering lipid membranes. Peptides belonging to this group typically display an overall positive charge ranging from +2 to +11 and usually kill microbes, such as Gram-negative bacteria, by creating membrane defects, leading to a loss of gradients in electrolytes, signal substances and other factors.

In some embodiments, the present AMPs are “AMP-like” peptides including phage lytic agents referred to herein as Chlamydia phage (Chp) peptides or amurin peptides. The amurin peptides of the present disclosure are distinguishable from amurins. As is known in the art, amurins, which are obtained from ssDNA or ssRNA phages (Microviridae and Leviviridae, respectively), are integral membrane proteins with a putative domain structure including an internal LS dipeptide immediately preceded by a stretch of 10-17 hydrophobic residues. Examples of amurins include the protein E amurin from phage <pX174 (Family Microviridae, genus Microvints), which is a 91 amino acid membrane protein that causes lysis by inhibiting the bacterial translocase Mra Y, an essential membrane-embedded enzyme that catalyzes the formation of the murein precursor, Lipid I; the A2 capsid protein of phage Q˜ (Family Leviviridae, genus Allolevivirus), which is a 420-amino acid structural protein that causes lysis by interfering with MurA activity and dysregulating the process of peptidoglycan biosynthesis; the protein L amurin of phage MS2 (Family Levivirdae, genus Levivirus), which is a 75 amino acid integral membrane protein that causes lysis using a mechanism that requires the activity of host chaperone DnaJ. Typically, amurins cannot be purified and are not suitable for use as antibacterial therapeutics.

In contrast to amurins, the amurin peptides of the present disclosure are small cationic peptides with predicted α-helical structures similar to those of AMPs obtained from the innate immune systems of a variety of vertebrates (but with amino acid sequences dissimilar to AMPs). Amurin peptides are primarily found in Chlamydiamicroviruses and, to a lesser extent, in other related members of the subfamily Gokushovirinae. The amurin peptides from a variety of Microviridae phages exhibit 30-100% identity to each other and have no homology with other peptides. Unlike the amurins of Microviridae, which have cytoplasmic targets in the cell wall biosynthetic apparatus, and, accordingly, may not be easily accessed by externally applied proteins, the present amurin peptides can be used in purified form to exert bactericidal activity “from without.”

In some embodiments, the AMPs of the present disclosure include synthetic peptides. In some embodiments, the synthetic peptide reduces the minimum inhibitory concentration (MIC) of an antibiotic, which prevents visible growth of bacterium, but does not itself exhibit antibacterial activity. A particularly desirable synthetic peptide for use with the lysin-AMP polypeptide constructs of the present disclosure includes the FIRL peptidomimetic (SEQ ID NO: 126) (SEQ ID NO: 114 of PCT Publication No. WO 2020/046747). Without being limited by theory, FIRL (SEQ ID NO: 126) (SEQ ID NO: 114 of PCT Publication No. WO 2020/046747), which is related to a sequence of a protein involved in outer membrane protein biogenesis, BamD, appears to increase the permeability of the outer membrane to antibiotics. Further information regarding the proposed mechanism is found, for example, in Mori et al., Journal of Antimicrobial Chemotherapy, 2012, 67: 2173-2181, which is herein incorporated by reference in its entirety.

### Structure Stabilizing Components

In some embodiments, the lysin-AMP polypeptide constructs of the present disclosure further include at least one structure stabilizing component to maintain at least a portion of the structure of the first and/or second component in the construct, e.g., the lysin and/or AMP, substantially the same as in the unconjugated lysin and/or AMP. In some embodiments, the stabilizing structure is a linker. Typically, the at least one structure stabilizing component, such as a linker enables the lysin and AMP to substantially preserve the three-dimensional structure of the first and/or second protein moieties, such that at least one biological activity of the lysin and/or AMP is retained.

Suitable linkers and structure stabilizing components are disclosed, for example, in PCT Publication Nos. WO 2019/191633 and 2020/046747, both of which are incorporated by referenced herein.

### Examples of Lysin-AMP Polypeptide Constructs

Examples of lysin-AMP polypeptides that are disclosed herein include, for example, those disclosed in PCT Publication Nos. WO 2019/191633 and 2020/046747, both of which are incorporated by referenced herein.

For instance, in some embodiments, the lysin-AMP polypeptide construct comprises a MIDR moiety (SEQ ID NO: 125), a FIRL moiety (SEQ ID NO: 126) and an NPTH moiety (SEQ ID NO: 127) introduced N-terminally to the GN202 lysin (SEQ ID NO: 128) to generate the GN370 lysin (SEQ ID NO: 149) or a polypeptide having lysin activity and having at least 80%, such as at least 85%, such as at least 90%, such as at least 95%, such as at least 98%, or such as at least 99% sequence identity to SEQ ID NO: 149.

Table 3, below, depicts specific examples of the lysins and lysin-AMP constructs described herein. The AMP portion of the construct is double-underlined for GN168 (SEQ ID NO: 129), GN176 (SEQ ID NO: 130), GN178 (SEQ ID NO: 131), GN370 (SEQ ID NO: 149), GN371 (SEQ ID NO: 150) and GN93 (SEQ ID NO: 158). For all other constructs, double underlines correspond to a lysin. Structure stabilizing components, such as linkers, are italicized and bolded. The purification tag for GN486 (SEQ ID NO: 160) is italicized, underlined and bolded. Single point mutations are bolded.

TABLE 3
Lysins and Lysin-AMP Constructs

| GN # | Polypeptide Sequence |
|---|---|
| GN168 | MRLKMARRRYRLPRRRSRRLESRTALRMHPRNRLRRIMRGGIRFTAGGTA |
| | GGRTSQRGIDLIKSFEGLRLSAYQDSVGVWTIGYGTTRGVTRYMTITVEQ |
| | AERMLSNDIQRFEPELDRLAKVPLNQNQWDALMSFVYNLGAANLASSTL |
| | LDLLNKGDYQGAADQFPHWVNAGGKRLDGLVKRRAAERALFLEPLS |
| | (SEQ ID NO: 129) |
| GN176 | MSFNVTPKFKRWQLYFRGRMWTAGGTAGGRTSQRGIDLIKSFEGLRLSA |
| | YQDSVGVWTIGYGTTRGVTRYMTITVEQAERMLSNDIQRFEPELDRLAK |
| | VPLNQNQWDALMSFVYNLGAANLASSTLLDLLNKGDYQGAADQFPHW |
| | VNAGGKRLDGLVKRRAAERALFLEPLS (SEQ ID NO: 130) |
| GN178 | MPPIESKLAGKKIKNLLISGLKGGSGSGSGSGSPRTSQRGIDLIKSFEGLRLS |
| | AYQDSVGVWTIGYGTTRGVTRYMTITVEQAERMLSNDIQRFEPELDRLA |
| | KVPLNQNQWDALMSFVYNLGAANLASSTLLDLLNKGDYQGAADQFPH |
| | WVNAGGKRLDGLVKRRAAERALFLEPLS (SEQ ID NO: 131) |
| GN217 | MTYTLSKRSLDNLKGVHPDLVAVVHRAIQLTPVDFAVIEGLRSVSRQKEL |
| | VAAGASKTMNSRHLTGHAVDLAAYVNGIHWDWPLYDAIAVAVKAAAK |
| | ELGVAIVWGGDWTTFKDGPHFELDRSKYR (SEQ ID NO: 132) |
| GN218 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAK |
| | ELGVAIVWGGDWTTEKDGPHEELDRSKYGGGSGGGGSGGGSRLKKIGKV |
| | LKWI (SEQ ID NO: 133) |
| GN223 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAK |
| | ELGVAIVWGGDWTTEKDGPHEELDRSKYRPPGGGSGGGGSGGGSSKKAS |
| | RKSFTKGAVKVHKKNVPTRVPMRGGIRL (SEQ ID NO: 134) |
| GN239 | MTYTLSKRSLDNLKGVHPDLVAVVHRAIQLTPVDFAVIEGLRSVSRQKEL |
| | VAAGASKTMNSRHLTGHAVDLAAYVNGIRWDWPLYDAIAVAVKAAAK |
| | ELGVAIVWGGDWTTFKDGPHFELDRSKYGGGSGGGGSGGGSRKKTRKR |
| | LKKIGKVLKWI (SEQ ID NO: 135) |
| GN243 | MTYTLSKRSLDNLKGVHPDLVAVVHRAIQLTPVDEAVIEGLRSVSRQKEL |
| | VAAGASKTMNSRHLTGHAVDLAAYVNGIRWDWPLYDAIAVAVKAAAK |
| | ELGVAIVWGGDWTTFKDGPHFELDRSKYRKKTRKRLKKIGKVLKWIPPG |
| | GGSGGGGSGGGSTRKRLKKIGKVLKWI (SEQ ID NO: 136) |
| GN280 | MKLSEKRALFTQLLAQLILWAGTQDRVSVALDQVKRTQAEADANAKSG |
| | AGIRNSLHLLGLAGDLILYKDGKYMDKSEDYKFLGDYWKSLHPLCRWG |
| | GDFKSRPDGNHESLEHEGVQRKKTRKRLKKIGKVLKWIPPTAGGTAGGT |
| | RKRLKKIGKVLKWI (SEQ ID NO: 137) |
| GN281 | MKLSEKRALFTQLLAQLILWAGTQDRVSVALDQVKRTQAEADANAKSG |
| | AGIRNSLHLLGLAGDLILYKDGKYMDKSEDYKFLGDYWKSLHPLCRWG |
| | GDFKSRPDGNHESLEHEGVQRKKTRKRLKKIGKVLKWIGGGSGGGGSGG |
| | GSPPTRKRLKKIGKVLKWI (SEQ ID NO: 138) |
| GN316 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGFNCVICGYDNAE |
| | EMFNDFLTGERAQLMAFVKFIKADANLWKALKDKNWAEFARRYNGPAY |
| | AQNQYDTKLAAAYKSFS (SEQ ID NO: 139) |
| GN329 | MITDREYQQAAEMLGVDVPAIKAVTKVEAPVGGFQPTGEPTILYERHQM |
| | YRQLQAKGLPTEGHPPDLVNKVAGGYGKYSEQHAKLARAVKIDRDSALE |
| | SCSWGMFQIMGYHWKLMGYPTLQAFVNAMYASEGAQMDAFCRFIKAQP |
| | TTHAALKAHDWAKFARLYNGPGYAKNKYDVKLEKAYAEASG (SEQ ID |
| | NO: 140) |
| GN333 | MALTEQDFQSAADDLGVDVASVKAVTKVESRGSGFLLSGVPKILFERHW |
| | MFKLLKRKLGRDPEINDVCNPKAGGYLGGQAEHERLDKAVKMDRDCAL |
| | QSASWGLFQIMGFHWEALGYASVQAFVNAQYASEGSQLNTFVRFIKTNP |
| | AIHKALKSKDWAEFARRYNGPDYKKNNYDVKLAEAYQSFK (SEQ ID NO: |
| | 141) |
| GN349 | MAILKIGSKGLEVKNLQTSLNKIGENLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMENDELTGERAQLMAFVKFIKADANLWKALKDKNWAEFARRYNGPAY |
| | AQNQYDTKLAAAYKSFSTAGGTAGGARRYRLSRRRSRRLFSRTALRMHR |
| | RNRLRRIMRGGIRF (SEQ ID NO: 142) |
| GN351 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMFNDFLTGERAQLMAFVKFIKADANLWKALKDKNWAEFARRYNGPAY |
| | AQNQYDTKLAAAYKSFSTAGGTAGGARSRRRMSKRSSRRSFRKYAKSHK |
| | KNFKARSMRGGIRL (SEQ ID NO: 143) |
| GN352 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGETKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMENDELTGERAQLMAFVKFIKADANLWKALKDKNWAEFARRYNGPAY |
| | AQNQYDTKLAAAYKSESTAGGTAGGKRRKMTRKGSKRLFTATADKTKSI |
| | NTAPPPMRGGIRL (SEQ ID NO: 144) |
| GN353 | MAILKIGSKGLEVKNLQTSLNKIGENLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMENDELTGERAQLMAFVKFIKADANLWKALKDKNWAEFARRYNGPAY |
| | AQNQYDTKLAAAYKSESTAGGTAGGRKRMSKRVDKKVFRRTAASAKKI |
| | NIDPKIYRGGIRL (SEQ ID NO: 145) |
| GN357 | MAILKIGSKGLEVKNLQTSLNKIGENLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMFNDFLTGERAQLMAFVKFIKADANLWKALKDKNWAEFARRYNGPAY |
| | AQNQYDTKLAAAYKSESTAGGTAGGRRLIRLWLRLLR (SEQ ID NO: 146) |
| GN359 | MAILKIGSKGLEVKNLQTSLNKIGENLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMFNDFLTGERAQLMAFVKFIKADANLWKALKDKNWAEFARRYNGPAY |
| | AQNQYDTKLAAAYKSESTAGGTAGGTRKRLKKIGKVLKWI (SEQ ID NO: |
| | 147) |
| GN369 | MAILKIGSKGLEVKNLQTSLNKIGENLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGENCVICGYDNAE |
| | EMFNDELTGERAQLMAFVKFIKADANLWKALKDKNWAEFARRYNGPAY |
| | AQNQYDTKLAAAYKSFSRKKTRKRLKKIGKVLKWI (SEQ ID NO: 148) |
| GN370 | MIDRFIRLNPTHGPRRPRRPGRRAPVRTSQRGIDLIKSFEGLRLSAYQDSVG |
| | VWTIGYGTTRGVTRYMTITVEQAERMLSNDIQRFEPELDRLAKVPLNQNQ |
| | WDALMSFVYNLGAANLASSTLLDLLNKGDYQGAADQFPHWVNAGGKR |
| | LDGLVKRRAAERALFLEPLS (SEQ ID NO: 149) |
| GN371 | MIDRFIRLNPTHRTSQRGIDLIKSFEGLRLSAYQDSVGVWTIGYGTTRGVTR |
| | YMTITVEQAERMLSNDIQRFEPELDRLAKVPLNQNQWDALMSFVYNLGA |
| | ANLASSTLLDLLNKGDYQGAADQFPHWVNAGGKRLDGLVKRRAAERAL |
| | FLEPLS (SEQ ID NO: 150) |
| GN394 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGFNCVICGYDNAE |
| | EMFNDFLTGERAQLMAFVDFIKADANLWKALKDKNWAEFARRYNGPAY |
| | AQNQYDTKLAAAYKSFS (SEQ ID NO: 151) |
| GN396 | MAILKIGSKGLEVKNLQTSLNKIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGFNCVICGYDNAE |
| | EMFNDFLTGERAQLMAFVKFIKADANLWDALKDKNWAEFARRYNGPAY |
| | AQNQYDTKLAAAYKSFS (SEQ ID NO: 152) |
| GN408 | MAILKIGSKGLEVKNLQTSLNKIGFNLVADGIFGKATDNAVRAVQAGAGL |
| | VVDGIAGPKTMYAIRNAGESHQDHLTEADLIDAAHELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGFNCVICGYDNAE |
| | EMFNDFLTGERAQLMAFVKFIKADANLWKALKDKNWAEFARRYNGPAY |
| | AQNQYDTKLAAAYKSFS(SEQ ID NO: 153) |
| GN418 | MAILKIGSKGLEVKNLQTSLNDIGFNLVADGIFGKATDNAVRAVQAGAGL |
| | VVDGIAGPKTMYAIRNAGESHQDHLTEADLIDAARELSVDLASIKAVNQV |
| | ESRGTGFTKSGKIKTLFERHIMYKKLNAKFGQAKANALAQLYPTLVNAK |
| | AGGYTGGDAELERLHGAIAIDKDCAYESASYGLFQIMGFNCVICGYDNAE |
| | EMFNDFLTGERAQLMAFVKFIKADANLWKALKDKNWAEFARRYNGPAY |
| | AQNQYDTKLAAAYKSFS (SEQ ID NO: 154) |
| GN424 | MNTLRFNSRGAEVGVLQQRLVRAGYPIDVTHLYDEATEQAVKALQAAA |
| | GIVVDGIAGPNTYAVLSAGQRDRKHLTEADIARAADKLGVSPACVRAVN |
| | EVESRGSGFLADGRPVILFERHVMYNRLVAAKRAVDAASAAQRFPNVVS |
| | AKPGGYQGGAAEYVRLDTAARIDAAIAYESASWGAFQVMGYHWERLGY |
| | SSIDEFVARMETSEGEQLDAFVRFVAADSSLRTALKNRKWAAFAKGYNG |
| | PDYARNLYDAKLAQAYERYAGTKAAA (SEQ ID NO: 155) |
| GN425 | MTLRLDDVGLDVLHLQKRLNELGANPRLLPDGQFGEVTERAVRAFQQRA |
| | GLVVDGVAGPKTMAALSGHSTSRLLGQRDLQRAADRLGVPLASVMALN |
| | AVESRGEGFAANGRPVILFERHVMHERLQVNGLSEAEADALAARHPGLV |
| | SRRPGGYVGDTAEHQRLANARLLHDTAALESASWGLFQVMGYHWQAL |
| | GYDTTQDFTERMARHEAEHLEAFVRFIEADPALHKALKGRKWAEFARRY |
| | NGPAYARNLYDVKLARAFEQFSDALQAAA (SEQ ID NO: 156) |
| GN428 | MAILKLGNRGSEVKALQQSLNKIGFSLTADGIFGKATENAVKSVQAGAGL |
| | VIDGIAGPKTFYAIRNAGDAHQEHLTEADLVDAARELGVELASMKAVNQ |
| | VESRGTGFTKTGKIKTLFERHIMYKKVTAKFGQARANALYQLYPTLVNPN |
| | SGGYIGGDAELERLQGAIALDEDCAYESASYGLFQIMGFNCQICGYSNAK |
| | EMFTDFLTGERAHLLAFVKFIKADANMWKALKNKNWAEFARRYNGPAY |
| | AKNQYDTKLAAAYKSFC (SEQ ID NO: 157) |
| GN93 | MKFFKFFKFFKAGAGAGAGAGAGAGAGASNNELPWVAEARKYIGLREDT |
| | SKTSHNPKLLAMLDRMGEFSNESRAWWHDDETPWCGLFVGYCLGVAGR |
| | YVVREWYRARAWEAPQLTKLDRPAYGALVTFTRSGGGHVGFIVGKDAR |
| | GNLMVLGGNQSNAVSIAPFAVSRVTGYFWPSFWRNKTAVKSVPFEERYS |
| | LPLLKSNGELSTNEA (SEQ ID NO: 158) |
| GN431 | MAILKLGNRGTEVKALQDSLNKIGFTLVADGIFGKATENAVKTVQAGAG |
| | LVIDGIVGPKTSYAIRNAGEAHQDHLTEADLIEAANQLGVDLASVKAVNQ |
| | VESRGTGFTKSGKIKTLFERHIMYKKLMAKFGQARANAMGQMYPTLVSP |
| | VAGGYTGGDAELDRLHAAINIDEDCAYESASYGLFQIMGFNCQVCGYAN |
| | AKEMFNDFLTGERAHLMAFVKFIKADAKLWQALKDKNWAEFARRYNGP |
| | AYTKNQYDTKLAAAYNSFN (SEQ ID NO: 159) |
| GN486 | MGSHHHHHHGGPRRPRRPGRRAPVRTSQRGIDLIKSFEGLRLSAYQDSV |
| | GVWTIGYGTTRGVTRYMTITVEQAERMLSNDIQRFEPELDRLAKVPLNQN |
| | QWDALMSFVYNLGAANLASSTLLKLLNKGDYQGAADQFPRWVNAGGK |
| | RLDGLVKRRAAERALFLEPLS (SEQ ID NO: 160) |
| GN485 | MPGLSGFIRNADTPVTSLGSAGHVHVPEGPLIRINPDCLLGTPFKFFKFFKF |
| | FKFFKFFKFFKFFKNECVLL (SEQ ID NO: 161) |

In some embodiments, the lysins and/or lysin-AMP constructs of the present disclosure are chemically modified. A chemical modification includes but is not limited to, adding chemical moieties, creating new bonds, and removing chemical moieties. Chemical modifications can occur anywhere in a lysin and/or lysin-AMP construct, including the amino acid side chains, as well as the amino or carboxyl termini. For example, in certain embodiments, the lysin or lysin-AMP construct comprises an N-terminal acetylation modification. In certain embodiments, the lysin or lysin-AMP construct comprises a C-terminal amidation modification. Such modification can be present at more than one site in a lysin and/or lysin-AMP construct.

Furthermore, one or more side groups, or terminal groups of a lysin and/or lysin-AMP polypeptide construct may be protected by protective groups known to the person ordinarily-skilled in the art.

In some embodiments, the lysins and/or lysin-AMP constructs are conjugated to a duration enhancing moiety. In some embodiment, the duration enhancing moiety is polyethylene glycol. Polyethylene glycol (“PEG”) has been used to obtain therapeutic polypeptides of enhanced duration (Zalipsky, S., Bioconjugate Chemistry, 6:150-165 (1995); Mehvar, R., J. Pharm. Pharmaceut. Sci., 3:125-136 (2000), which is herein incorporated by reference in its entirety). The PEG backbone, (CH₂CH₂—O—)ₙ, wherein n is a number of repeating monomers, is flexible and amphiphilic. When attached to another chemical entity, such as a lysin and/or lysin-AMP construct, PEG polymer chains can protect such polypeptides from immune response and other clearance mechanisms. As a result, pegylation can lead to improved efficacy and safety by optimizing pharmacokinetics, increasing bioavailability, and decreasing immunogenicity and dosing amount and/or frequency.

### Polynucleotides

### Lysin, Variant Lysins, Active Fragments Thereof or Derivatives

In one aspect, the present disclosure is directed an isolated polynucleotide comprising a nucleic acid molecule encoding a Chp peptide, a lysin, a lysin-AMP construct, or a variant or active fragment thereof as described herein. In some embodiments, the isolated polynucleotide sequence is a DNA sequence. In other embodiments, the isolated polynucleotide is a cDNA sequence.

In some embodiments, the isolated polynucleotide comprises a nucleic acid molecule encoding a polypeptide having at least 80%, such as at least 85%, such as at least 90%, such as at least 95%, such as at least 98%, or such as at least 99% sequence identity with a Chp peptide, a lysin, a lysin-AMP construct, or a variant or active fragment thereof as described herein, wherein the encoded polypeptide inhibits the growth, or reduces the population, or kills P. aeruginosa and optionally at least one other species of Gram-negative bacteria as described herein in the absence or presence of, or in both the absence and presence of, human serum, or in the presence of pulmonary surfactant.

In some embodiments, the isolated polynucleotide comprises a nucleic acid molecule encoding at least one of Chp2, Chp-M1, Chp10-M1, Unp2-M1, Chp4-M1, Chp6, and Chp6-M1, and a lysin-AMP construct selected from GN370 or a variant or an active fragment thereof or derivative, wherein the variant or an active fragment thereof or derivative encoded by the isolated polynucleotide inhibits the growth, or reduces the population, or kills P. aeruginosa and/or at least one other species of Gram-negative bacteria in the absence or presence of, or in both the absence and presence of, human serum, or in the presence of pulmonary surfactant.

In some embodiments, the isolated polynucleotide comprises a nucleic acid molecule encoding a Chp peptide, wherein the Chp peptide is at least one of Chp2, Chp-M1, Chp10-M1, Unp2-M1, Chp4-M1, Chp6, and Chp6-M1 or a nucleic acid molecule encoding a polypeptide having lysin activity and having at least 80%, such as at least 85%, such as at least 90%, such as at least 95%, such as at least 98%, or such as at least 99% sequence identity to Chp2, Chp-M1, Chp10-M1, Unp2-M1, Chp4-M1, Chp6, and Chp6-M1.

In some embodiments, the isolated polynucleotide comprises a nucleic acid molecule encoding a lysin-AMP construct, wherein the lysin-AMP construct is GN370 or a nucleic acid molecule encoding a polypeptide having lysin activity and having at least 80%, such as at least 85%, such as at least 90%, such as at least 95%, such as at least 98%, or such as at least 99% sequence identity to GN370.

### Vectors and Host Cells

In another aspect, the present disclosure is directed to a vector comprising an isolated polynucleotide comprising a nucleic acid molecule encoding any of the Chp peptides, lyins, or lysin-AMP constructs or variants or active fragments thereof disclosed herein or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule that encodes a Chp peptide having an amino acid sequence selected from the group consisting of SEQ ID NOs: 1-26, 54-67, and 81-102 or active fragments thereof.

In some embodiments, the present disclosure is directed to a vector comprising a nucleic acid molecule that encodes a Chp peptide having an amino acid sequence selected from the group consisting of SEQ ID NO: 2, SEQ ID NO: 6, SEQ ID NO: 81, SEQ ID NO: 88, SEQ ID NO: 89, SEQ ID NO: 91, and SEQ ID NO: 97 or active fragments thereof.

In some embodiments, the present disclosure is directed to a vector comprising a nucleic acid molecule that encodes a lysin-AMP construct having an amino acid sequence selected from the group consisting of SEQ ID NO: 149 or active fragments thereof.

Generally, any system or vector suitable to maintain, propagate or express a polypeptide in a host may be used for expression of the Chp peptides, lysins, or lysin-AMP constructs disclosed herein or active fragments thereof. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the Chp peptides or active fragments thereof to provide convenient methods of isolation, e.g., c-myc, biotin, poly-His, etc. Kits for such expression systems are commercially available.

A wide variety of host/expression vector combinations may be employed in expressing the polynucleotide sequences encoding the Chp peptides, lysins, or lysin-AMP constructs disclosed herein or active fragments thereof. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the Chp peptides, lysins, lysin-AMP constructs, or active fragments thereof of the present disclosure. Suitable vectors include but are not limited to derivatives of SV40 and known bacterial plasmids, e.g., E. coli plasmids colEl, pCRl, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the Chp peptides, lysins, or lysin-AMP constructs or active fragments thereof of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the Chp peptides, lysins, or lysin-AMP constructs or active fragments thereof are operatively linked to a heterologous promoter or regulatory element.

In another aspect, the present disclosure is directed to a host cell comprising any of the vectors disclosed herein including the expression vectors comprising the polynucleotide sequences encoding the Chp peptides, lysins, or lysin-AMP constructs or active fragments thereof of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSCl, BSC40, and BMT10), insect cells (e.g., Sf9), and human cells and plant cells in tissue culture. While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCSllO (Agilent Technologies, Inc.), JM109 (Promega, Inc.), LMG194 (ATCC), and BL21 (Thermo Fisher Scientific, Inc.).

There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in detail by Rosano, G. and Ceccarelli, E., Front Microbial., 5: 172 (2014).

Efficient expression of the present Chp peptides, lysins, or lysin-AMP constructs or active fragments thereof depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding Chp peptides, lysins, or lysin-AMP constructs or active fragments thereof of the present disclosure, one skilled in the art will be able to select the proper vectors, expression control sequences, and hosts without undue experimentation to accomplish the desired expression without departing from the scope of this disclosure.

Chp peptides, lysins, or lysin-AMP constructs or active fragments thereof of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also be employed for Chp peptide purification.

Alternatively, the vector system used for the production of Chp peptides, lysins, or lysin-AMP constructs or active fragments of the present disclosure may be a cell-free expression system. Various cell-free expression systems are commercially available, including, but are not limited to those available from Promega, LifeTechnologies, Clonetech, etc.

As indicated above, there is an array of choices when it comes to protein production and purification. Examples of suitable methods and strategies to be considered in protein production and purification are provided in WO 2017/049233, which is herein incorporated by reference in its entirety and further provided in Structural Genomics Consortium et al., Nat. Methods., 5(2): 135-146 (2008).

### Pharmaceutical Compositions

The compositions of the present disclosure can take the form of solutions, suspensions, emulsion, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

Administration of the compositions of the present disclosure or pharmaceutically acceptable forms thereof may be topical, i.e., the pharmaceutical composition may be applied directly where its action is desired (for example directly to a wound), or systemic. In turn, systemic administration can be enteral or oral, i.e., the composition may be given via the digestive tract, parenteral, i.e., the composition may be given by other routes than the digestive tract such as by injection or inhalation. Thus, the Chp peptides, lysins, or lysin-AMP constructs of the present disclosure or active fragments thereof and compositions comprising them can be administered to a subject orally, parenterally, by inhalation, topically, rectally, nasally, buccally, via an implanted reservoir, or by any other known method. The Chp peptides, lysins, or lysin-AMP constructs of the present disclosure or active fragments thereof can also be administered by means of sustained release dosage forms.

For oral administration, the Chp peptides, lysins, or lysin-AMP constructs of the present disclosure or active fragments thereof can be formulated into solid or liquid preparations, for example tablets, capsules, powders, solutions, suspensions, and dispersions. The composition can be formulated with excipients such as, e.g., lactose, sucrose, corn starch, gelatin, potato starch, alginic acid, and/or magnesium stearate.

For preparing solid compositions such as tablets and pills, a Chp peptide, lysin, or lysin-AMP construct of the present disclosure or active fragments thereof may be mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The topical compositions of the present disclosure may further comprise a pharmaceutically or physiologically acceptable carrier, such as a dermatologically or an otically acceptable carrier. Such carriers, in the case of dermatologically acceptable carriers, may be compatible with skin, nails, mucous membranes, tissues, and/or hair, and can include any conventionally-used dermatological carrier meeting these requirements. In the case of otically acceptable carriers, the carrier may be compatible with all parts of the ear. Such carriers can be readily selected by one of ordinary skill in the art. Carriers for topical administration of the compositions of the present disclosure include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated, for example, in an oleaginous hydrocarbon base, an anhydrous absorption base, a water-in-oil absorption base, an oil-in-water water-removable base, and/or a water-soluble base. In formulating otic compositions, the active components of the present disclosure may be formulated, for example, in an aqueous polymeric suspension including such carriers as dextrans, polyethylene glycols, polyvinylpyrrolidone, polysaccharide gels, gellan gums such as Gelrite®, cellulosic polymers such as hydroxypropyl methylcellulose, and carboxy-containing polymers such as polymers or copolymers of acrylic acid, as well as other polymeric demulcents. The topical compositions according to the present disclosure may be in any form suitable for topical application, including aqueous, aqueous-alcoholic or oily solutions; lotion or serum dispersions; aqueous, anhydrous or oily gels; emulsions obtained by dispersion of a fatty phase in an aqueous phase (O/W or oil-in-water) or, conversely, (W/O or water-in-oil); microemulsions or alternatively microcapsules, microparticles or lipid vesicle dispersions of ionic and/or nonionic type; creams; lotions; gels; foams (which may use a pressurized canister, a suitable applicator, an emulsifier, and an inert propellant); essences; milks; suspensions; and patches. Topical compositions of the present disclosure may also contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. In a further aspect, the topical compositions disclosed herein may be administered in conjunction with devices such as transdermal patches, dressings, pads, wraps, matrices, and bandages capable of being adhered to or otherwise associated with the skin or other tissue of a subject, being capable of delivering a therapeutically effective amount of one or more Chp peptide, lysin, or lysin-AMP construct or active fragment thereof as disclosed herein.

In one embodiment, the topical compositions of the present disclosure additionally comprise one or more components used to treat topical burns. Such components may include, but are not limited to, a propylene glycol hydrogel; a combination of a glycol, a cellulose derivative, and a water soluble aluminum salt; an antiseptic; an antibiotic; and a corticosteroid. Humectants such as solid or liquid wax esters; absorption promoters such as hydrophilic clays or starches; viscosity building agents; and skin-protecting agents may also be added. Topical formulations may be in the form of rinses such as mouthwash. See, e.g., WO 2004/004650.

The compositions of the present disclosure may also be administered by injection of a therapeutic agent comprising the appropriate amount of a Chp peptide, lysin, or lysin-AMP construct or active fragment thereof and a carrier. For example, the Chp peptide, lysin, or lysin-AMP construct or active fragment thereof can be administered intramuscularly, intrathecally, subdermally, subcutaneously, or intravenously to treat infections by Gram-negative bacteria, such as those caused by P. aeruginosa, and/or infections by acid-fast bacteria, such as those caused by species of actinobacteria, including, for example, M. tuberculosis and non-tuberculosis mycobacteria. The carrier may be comprised of distilled water, a saline solution, albumin, a serum, or any combinations thereof. Additionally, pharmaceutical compositions of parenteral injections can comprise pharmaceutically acceptable aqueous or nonaqueous solutions of Chp peptides, lysins, or lysin-AMP constructs as disclosed herein or active fragments thereof in addition to one or more of the following: pH buffered solutions, adjuvants (e.g., preservatives, wetting agents, emulsifying agents, and dispersing agents), liposomal formulations, nanoparticles, dispersions, suspensions or emulsions, as well as sterile powders for reconstitution into sterile injectable solutions or dispersions just prior to use.

In cases where parenteral injection is the chosen mode of administration, an isotonic formulation may be used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application may be provided sterile and pyrogen free.

The diluent may further comprise one or more other excipient such as ethanol, propylene glycol, an oil, or a pharmaceutically acceptable emulsifier or surfactant.

In another embodiment, the compositions of the present disclosure are inhalable compositions. The inhalable compositions of the present disclosure can further comprise a pharmaceutically acceptable carrier. In one embodiment, the Chp peptides, lysins, or lysin-AMP constructs of the present disclosure or active fragments thereof may be formulated as a dry, inhalable powder. In specific embodiments, an inhalation solution comprising Chp peptides, lysins, or lysin-AMP constructs or active fragments thereof may further be formulated with a propellant for aerosol delivery. In certain embodiments, solutions may be nebulized.

A surfactant can be added to an inhalable pharmaceutical composition of the present disclosure in order to lower the surface and interfacial tension between the medicaments and the propellant. Where the medicaments, propellant and excipient are to form a suspension, a surfactant may or may not be used. Where the medicaments, propellant and excipient are to form a solution, a surfactant may or may not be used, depending, for example, on the solubility of the particular medicament and excipient. The surfactant may be any suitable, non-toxic compound which is non-reactive with the medicament and which reduces the surface tension between the medicament, the excipient and the propellant and/or acts as a valve lubricant.

Examples of suitable surfactants include, but are not limited to: oleic acid; sorbitan trioleate; cetyl pyridinium chloride; soya lecithin; polyoxyethylene (20) sorbitan monolaurate; polyoxyethylene (10) stearyl ether; polyoxyethylene (2) oleyl ether; polyoxypropylene-polyoxyethylene ethylene diamine block copolymers; polyoxyethylene (20) sorbitan monostearate; polyoxyethylene(20) sorbitan monooleate; polyoxypropylene-polyoxyethylene block copolymers; castor oil ethoxylate; and combinations thereof.

Examples of suitable propellants include, but are not limited to: dichlorodifluoromethane, trichlorofluoromethane, dichloro-tetrafluoroethane, and carbon dioxide.

Examples of suitable excipients for use in inhalable compositions include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the compositions of the present disclosure comprise nasal applications. Nasal applications include applications for direct use, such as nasal sprays, nasal drops, nasal ointments, nasal washes, nasal injections, nasal packings, bronchial sprays and inhalers, as well as applications for indirect use, such as throat lozenges and mouthwashes or gargles, or through the use of ointments applied to the nasal nares or the face, and any combination of these and similar methods of application.

In another embodiment, the pharmaceutical compositions of the present disclosure comprise a complementary agent, including one or more antimicrobial agents and/or one or more conventional antibiotics. In order to accelerate the treatment of the infection, or augment the antibacterial effect, the therapeutic agent containing a Chp peptide, lysin, or lysin-AMP construct of the present disclosure or active fragment thereof may further include at least one complementary agent that can also potentiate the bactericidal activity of the peptide. The complementary agent may be one or more antibiotics used to treat Gram-negative bacteria or one or more antibiotics used to treat acid-fast bacteria. In one embodiment, the complementary agent is an antibiotic or antimicrobial agent used for the treatment of infections caused by P. aeruginosa, including MDR and/or XDR strains of P. aeruginosa. In one embodiment, the complementary agent is an antibiotic or antimicrobial agent used for the treatment of infections caused by M. tuberculosis, and in one embodiment, the complementary agent is an antibiotic or antimicrobial agent used for the treatment of infections caused by non-tuberculosis mycobacteria.

The compositions of the present disclosure may be presented in unit dosage form and may be prepared by any methods well known in the art. The amount of active ingredients that can be combined with a carrier material to produce a single dosage form will vary depending, for example, upon the host being treated, the duration of exposure of the recipient to the infectious bacteria, the size and weight of the subject, and the particular mode of administration. The amount of active ingredients that can be combined with a carrier material to produce a single dosage form may, for example, be that amount of each compound which produces a therapeutic effect. In certain embodiments, out of one hundred percent, the total amount may range from about 1 percent to about ninety-nine percent of active ingredients, such as from about 5 percent to about 70 percent, or from about 10 percent to about 30 percent.

### Dosage and Administration

Dosages administered may depend on a number of factors such as the activity of infection being treated; the age, health and general physical condition of the subject to be treated; the activity of a particular Chp peptide, lysin, or lysin-AMP construct or active fragment thereof; the nature and activity of the antibiotic if any with which a Chp peptide, lysin, or lysin-AMP construct or active fragment thereof according to the present disclosure is being paired; and the combined effect of such pairing. In certain embodiments, effective amounts of the Chp peptide, lysin, or lysin-AMP construct or active fragment thereof to be administered may fall within the range of about 1-50 mg/kg (or 1 to 50 mcg/ml). In certain embodiments, effective amounts of the Chp peptide, lysin, or lysin-AMP construct or active fragment thereof to be administered may fall within the range of about 1-50 μg/mL, such as within the range of about 1-10 μg/mL, about 1 g/mL, or about 10 μg/mL. In certain embodiments, the Chp peptide, lysin, or lysin-AMP construct or active fragment thereof may be administered 1-4 times daily for a period ranging from 1 to 14 days. The antibiotic if one is also used may be administered at standard dosing regimens or in lower amounts in view of any synergism. All such dosages and regimens, however, (whether of the Chp peptide, lysin, or lysin-AMP construct or active fragment thereof or any antibiotic administered in conjunction therewith) are subject to optimization. Optimal dosages can be determined by performing in vitro and in vivo pilot efficacy experiments as is within the skill of the art but taking the present disclosure into account.

It is contemplated that the Chp peptides, lysins, or lysin-AMP constructs disclosed herein or active fragments thereof may provide a rapid bactericidal and, when used in sub-MIC amounts, may provide a bacteriostatic effect. It is further contemplated that the Chp peptides, lysins, or lysin-AMP constructs disclosed herein or active fragments thereof may be active against a range of antibiotic-resistant bacteria and may not be associated with evolving resistance. Based on the present disclosure, in a clinical setting, the present Chp peptides, lysins, or lysin-AMP constructs or active fragments thereof may be a potent alternative (or additive) for treating infections arising from drug- and multidrug-resistant, and extensively drug-resistant bacteria alone or together with antibiotics (including antibiotics to which resistance has developed). It is believed that existing resistance mechanisms for Gram-negative bacteria do not affect sensitivity to the lytic activity of the present Chp peptides, lysins, or lysin-AMP constructs or active fragments thereof.

In some embodiments, time exposure to the Chp peptides, lysins, or lysin-AMP constructs disclosed herein or active fragments thereof may influence the desired concentration of active peptide units per ml. Carriers that are classified as “long” or “slow” release carriers (such as, for example, certain nasal sprays or lozenges) may possess or provide a lower concentration of peptide units per ml but over a longer period of time, whereas a “short” or “fast” release carrier (such as, for example, a gargle) may possess or provide a high concentration peptide units (mcg) per ml but over a shorter period of time. There are circumstances where it may be desirable to have a higher unit/ml dosage or a lower unit/ml dosage.

For the Chp peptides, lysins, or lysin-AMP constructs or active fragments thereof of the present disclosure, the therapeutically effective dose may be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration, in humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

A treatment regimen can entail administration daily (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration may be used. Dosage and frequency may vary depending on the patient. It will be understood by one of skill in the art that such guidelines will be adjusted for localized administration, e.g., intranasal, inhalation, rectal, etc., or for systemic administration, e.g., oral, rectal (e.g., via enema), intramuscular (i.m.), intraperitoneal (i.p.), intravenous (i.v.), subcutaneous (s.c.), transurethral, and the like.

### Methods

The Chp peptides, lysins, lysin-AMP constructs and active fragments thereof of the present disclosure can be used in vivo, for example, to treat bacterial infections due to Gram-negative bacteria, such as P. aeruginosa, or due to acid-fast bacteria, such as actinobacteria, in a subject, as well as in vitro, for example to reduce the level of bacterial contamination on, for example, a surface, e.g., of a medical device. In certain embodiments, the Gram-negative bacteria is resistant to at least one antibiotic or is an MDR pathogen. In certain embodiments, the Gram-negative bacteria is resistant to multiple antibiotics or is an XDR pathogen.

For example, in some embodiments, the present Chp peptides, lysins, or lysin-AMP constructs or active fragments thereof may be used for the prevention, disruption, and/or eradication of bacterial biofilm formed by Gram-negative bacteria or acid-fast bacteria. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as the mucus plugs of the lung (such as the lung of a cystic fibrosis patient), contaminated catheters, contact lenses, etc (Sharma et al. Biologicals, 42(1):1-7 (2014), which is herein incorporated by reference in its entirety). Thus, in one embodiment, the Chp peptides, lysins, or lysin-AMP constructs or active fragments thereof of the present disclosure can be used for the prevention, disruption, and/or eradication of bacterial infections due to Gram-negative bacteria or acid-fast bacteria when the bacteria are protected by a bacterial biofilm. In one embodiment, Chp2 or an active fragment thereof can be used for the prevention, disruption, and/or eradication of bacterial infections due to a Gram-negative bacteria when the bacteria are protected by a bacterial biofilm. In one embodiment, Chp2-M1 or an active fragment thereof can be used for the prevention, disruption, and/or eradication of bacterial infections due to a Gram-negative bacteria when the bacteria are protected by a bacterial biofilm. In one embodiment, Chp10-M1 or an active fragment thereof can be used for the prevention, disruption, and/or eradication of bacterial infections due to a Gram-negative bacteria when the bacteria are protected by a bacterial biofilm.

In certain embodiments, GN370, Chp2, Chp2-M1, and/or Chp10-M1, or an active fragment thereof, can be used for the prevention, disruption, and/or eradication of bacterial infections due to P. aeruginosa (including mucoid, non-mucoid and/or hemolytic (such as α-hemolytic) isolates, MDR isolates, and/or XDR isolates), B. multiovorans, an Achromobacter species, such as A. xylosoxidans, and/or a Stenotrophomonas species, such as S. maltophilia, wherein the bacteria are protected by a bacterial biofilm. In another embodiment, GN370, Chp2, Chp2-M1, and/or Chp10-M1, or an active fragment thereof, can be used for the prevention, disruption, and/or eradication of bacterial infections due to P. aeruginosa (including mucoid, non-mucoid and/or hemolytic (such as α-hemolytic) isolates, MDR isolates, and/or XDR isolates), Stenotrophomonas maltophilia, Achromobacter xylosoxidans, Achromobacter ruhlandii and Achromobacter dolens. In still another embodiment, GN370, Chp2, Chp2-M1, and/or Chp10-M1, or an active fragment thereof, can be used for the prevention, disruption, and/or eradication of bacterial infections due to P. aeruginosa (including mucoid, non-mucoid and/or hemolytic (such as α-hemolytic) isolates, MDR isolates, and/or XDR isolates), Stenotrophomonas maltophilia, Achromobacter xylosoxidans. In still another embodiment, GN370, Chp2, Chp2-M1, and/or Chp10-M1, or an active fragment thereof, can be used for the prevention, disruption, and/or eradication of bacterial infections due to P. aeruginosa (including mucoid, non-mucoid and/or hemolytic (such as α-hemolytic) isolates, MDR isolates, and/or XDR isolates) and Stenotrophomonas maltophilia, and in still another embodiment, GN370, Chp2, Chp2-M1, and/or Chp10-M1, or an active fragment thereof, can be used for the prevention, disruption, and/or eradication of bacterial infections due to P. aeruginosa (including mucoid, non-mucoid and/or hemolytic (such as α-hemolytic) isolates, MDR isolates, and/or XDR isolates). In certain embodiments, GN370, Chp2, Chp2-M1, and/or Chp10-M1 or an active fragment thereof can eradicate Gram-negative bacterial biofilm, such as an A. xylosoxidans, S. maltophilia, P. aeruginosa, and/or B. multiovorans bacterial biofilm. In another embodiment, GN370, Chp2, Chp2-M1, and/or Chp10-M1 or an active fragment thereof can eradicate Gram-negative bacterial biofilm, such as A. xylosoxidans, S. maltophilia, P. aeruginosa (including mucoid, non-mucoid and/or hemolytic (such as α-hemolytic) isolates, MDR isolates, and/or XDR isolates). In still another embodiment, GN370, Chp2, Chp2-M1, and/or Chp10-M1 or an active fragment thereof can eradicate Gram-negative bacterial biofilm, such as an S. maltophilia and P. aeruginosa (including mucoid, non-mucoid and/or hemolytic (such as α-hemolytic) isolates, MDR isolates, and/or XDR isolates). In still another embodiment, GN370, Chp2, Chp2-M1, and/or Chp10-M1 or an active fragment thereof can eradicate Gram-negative bacterial biofilm, such as an P. aeruginosa (including mucoid, non-mucoid and/or hemolytic (such as α-hemolytic) isolates, MDR isolates and/or XDR isolates). In one embodiment, Chp2 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates), S. maltophilia, A. xylosoxidans, and/or B. multiovorans. In another embodiment, Chp2 can prevent, disrupt, or eradicate a Gram-negative bacterial biofilm comprising S. maltophilia and/or P. aeruginosa (including mucoid, non-mucoid and/or hemolytic (such as α-hemolytic) isolates, MDR isolates, and/or XDR isolates). In still another embodiment, Chp2 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates). In one embodiment, Chp2-M1 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates) and/or S. maltophilia. In one embodiment, Chp10-M1 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates) and/or A. xylosoxidans. In one embodiment, GN370 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates) and/or S. maltophilia. In still another embodiment, GN370 can prevent, disrupt, or eradicate a Gram-negative bacterial biofilm comprising P. aeruginosa (including mucoid, non-mucoid and/or hemolytic (such as α-hemolytic) isolates, MDR isolates, and/or XDR isolates).

In one aspect, the present disclosure is directed to a method of treating a bacterial infection caused by one or more additional Gram-negative bacteria as described herein, comprising administering to a subject diagnosed with, at risk for, or exhibiting symptoms of a bacterial infection, a pharmaceutical composition as described herein described. In one aspect, the present disclosure is directed to a method of treating a bacterial infection caused by one or more additional acid-fast bacteria as described herein, comprising administering to a subject diagnosed with, at risk for, or exhibiting symptoms of a bacterial infection, a pharmaceutical composition as described herein described. In one aspect, the present disclosure is directed to a method of treating a bacterial infection caused by one or more MDR and/or XDR Gram-negative bacteria as described herein, comprising administering to a subject diagnosed with, at risk for, or exhibiting symptoms of a bacterial infection, a pharmaceutical composition as described herein described.

The terms “infection” and “bacterial infection” are meant to include respiratory tract infections (RTIs), such as respiratory tract infections in patients having cystic fibrosis (CF), including (acute) pulmonary exacerbations in CF patients and/or patients with decline in lung function and mortality; lower respiratory tract infections, such as acute exacerbation of chronic bronchitis (ACEB); acute sinusitis; community-acquired pneumonia (CAP); hospital-acquired pneumonia (HAP); ventilator-acquired pneumonia; cystic fibrosis-associated pneumonia; nosocomial respiratory tract infections; non-cystic fibrosis lung diseases such as bronchiectasis and/or acute pneumonias; sexually transmitted diseases, such as gonococcal cervicitis and gonococcal urethritis; urinary tract infections; acute otitis media; sepsis including neonatal septisemia and catheter-related sepsis; osteomyelitis; tuberculosis;, and non-tuberculosis mycobacteria infections. Infections caused by drug-resistant bacteria, multidrug-resistant bacteria, and extensively drug-resistant bacteria are also contemplated.

Non-limiting examples of infections caused by Gram-negative bacteria, such as P. aeruginosa, A. xylosoxidans, S. maltophilia, or acid-fast include: A) Nosocomial infections: 1. Respiratory tract infections especially in cystic fibrosis patients and mechanically-ventilated patients, including pulmonary exacerbations of cystic fibrosis patients and bronchiectasis in non-cystic fibrosis patients; 2. Bacteremia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections such as tuberculosis; 2. Meningitis; 3. Folliculitis and infections of the ear canal caused by contaminated water; 4. Malignant otitis externa in the elderly and diabetics; 5. Osteomyelitis of the calcaneus in children; 6. Eye infections commonly associated with contaminated contact lens; 7. Skin infections such as nail infections in people whose hands are frequently exposed to water; 8. Gastrointestinal tract infections; and 9. Musculoskeletal system infections.

The one or more species of Gram-negative bacteria of the present methods may include any of the species of Gram-negative bacteria as described herein. Typically, the additional species of Gram-negative bacteria are selected from one or more of Acinetobacter baumannii, Acinetobacter haemolyticus, Actinobacillus actinomycetemcomitans, Aeromonas hydrophila, Achromobacter spp., such as Achromobacter dolens, Achromobacter ruhlandii, and Achromobacter xylosoxidans, Bacteroides spp., such as, Bacteroides fragilis, Bacteroides theataioatamicron, Bacteroides distasonis, Bacteroides ovatus, and Bacteroides vulgatus, Bartonella Quintana, Bordetella pertussis, Brucella spp., such as, Brucella melitensis, Burkholderia spp, such as, Burkholderia anthina, Burkholderia cepacia, Burkholderia cenacepacia, Burkholderia gladioli, Burkholderia multivorans, Burkholderia pseudomallei, and Burkholderia mallei, Fusobacterium, Prevotella corporis, Prevotella intermedia, Prevotella endodontalis, Porphyromonas asaccharolytica, Campylobacter jejuni, Campylobacter fetus, Campylobacter coli, Chlamydia spp., such as Chlamydia pneumoniae and Chlamydia trachomatis, Citrobacter freundii, Citrobacter koseri, Coxiella burnetii, Edwarsiella spp., such as, Edwarsiella tarda, Eikenella corrodens, Enterobacter spp., such as, Enterobacter cloacae, Enterobacter aerogenes, Enterobacater faecium, and Enterobacter agglomerans, Escherichia coli, Francisella tularensis, Haemophilus influenzae, Haemophilus ducreyi, Helicobacter pylori, Kingella kingae, Klebsiella spp., such as, Klebsiella pneumoniae, Klebsiella oxytoca, Klebsiella rhinoscleromatis, and Klebsiella ozaenae, Kluyvera ascorbata, Legionella penumophila, Moraxella spp., such as, Moraxella catarrhalis, Morganella spp., such as, Morganella morganii, Neisseria gonorrhoeae, Neisseria meningitidis, Pandoraea apista, P. aeruginosa, Pasteurella multocida, Plesiomonas shigelloides, Proteus mirabilis, Proteus vulgaris, Proteus penneri, Proteus myxofaciens, Providencia spp., such as, Providencia stuartii, Providencia rettgeri, Providencia alcalifaciens, Pseudomonas fluorescens, Raoultella ornithinolytica, Salmonella typhi, Salmonella typhimurium, Salmonella paratyphi, Serratia spp., such as, Serratia marcescens, Shigella spp., such as, Shigella flexneri, Shigella boydii, Shigella sonnei, and Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydia pneumoniae, Chlamydia trachomatis, Ricketsia prowazekii, Coxiella burnetii, Ehrlichia chafeensis and/or Bartonella hensenae.

In a certain embodiment, the one or more species of Gram-negative bacteria of the present methods may include any of the species of Gram-negative bacteria selected from Pseudomonoas aeruginosa (including mucoid, non-mucoid and/or hemolytic (such as α-hemolytic) isolates), Stenotrophomonas maltophilia, Achromobacter xylosoxidans, Achromobacter ruhlandii, Achromobacter dolens, in particular embodiment, Pseudomonas aeruginosa, Stenotrophomonas maltophilia and Achromobacter xylosoxidans, in still another embodiment, Pseudomonas aeruginosa and Stenotrophomonas maltophilia, in still another embodiment, Pseudomonas aeruginosa (including mucoid, non-mucoid and/or hemolytic (such as α-hemolytic) isolates, particularly mucoid and alpha-hemolytic isolates). In certain embodiments of all aspects of the invention, the one or more species of Gram-negative bacteria may include mucoid and/or α-hemolytic isolates of P. aeruginosa, and/or MDR isolates and/or XDR isolates and/or carbapenem-resistant isolates of P. aeruginosa, S. maltophilia, and/or A. xylosoxidans.

More typically, the at least one other species of Gram-negative bacteria is selected from one or more of Acinetobacter baumannii, Bordetella pertussis, Burkholderia cepacia, Burkholderia pseudomallei, Burkholderia mallei, Campylobacter jejuni, Campylobacter coli, Enterobacter cloacae, Enterobacter aerogenes, Escherichia coli, Francisella tularensis, Haemophilus influenzae, Haemophilus ducreyi, Helicobacter pylori, Klebsiella pneumoniae, Legionella penumophila, Moraxella catarrhalis, Morganella morganii, Neisseria gonorrhoeae, Neisseria meningitidis, Pasteurella multocida, Proteus mirabilis, Proteus vulgaris, Salmonella typhi, Serratia marcescens, Shigella flexneri, Shigella boydii, Shigella sonnei, Shigella dysenteriae, Stenotrophomonas maltophilia, Vibrio cholerae, and/or Chlamydia pneumoniae.

Even more typically, the at least one other species of Gram-negative bacteria is selected from one or more of Stenotrophomonas spp. (e.g., Stenotrophomonas maltophilia), Salmonella typhimurium, Salmonella typhi, Shigella spp., Escherichia coli, Acinetobacter baumanii, Klebsiella pneumonia, Neisseria gonorrhoeae, Neisseria meningitides, Serratia spp. Proteus mirabilis, Morganella morganii, Providencia spp., Edwardsiella spp., Yersinia spp., Haemophilus influenza, Bartonella quintana, Brucella spp., Bordetella pertussis, Burkholderia spp., Moraxella spp., Francisella tularensis, Legionella pneumophila, Coxiella burnetii, Bacteroides spp., Enterobacter spp., and/or Chlamydia spp.

Yet even more typically, the at least one other species of Gram-negative bacteria is selected from one or more of Klebsiella spp., Enterobacter spp., Escherichia coli, Citrobacter freundii, Salmonella typhimurium, Yersinia pestis, Stenotrophomonas maltophilia, and/or Franciscella tulerensis.

The one or more species of acid-fast bacteria of the present methods may include any of the species of acid-fast bacteria as described herein. Typically, the additional species of acid-fast bacteria are selected from one or more species of actinobacteria, such as mycobacteria.

Mycobacteria are a family of small, rod-shaped bacilli that can be classified into 3 main groups for the purpose of diagnosis and treatment. The first is Mycobacterium tuberculosis complex which can cause pulmonary tuberculosis and includes M. tuberculosis, M. bovis, M. africanum, M. microti and M. canetti. The second group includes M. leprae and M. lepromatosis, which cause Hansen's disease or leprosy. The third group is nontuberculous mycobacteria (NTM), which include all the other mycobacteria that can cause lung disease resembling tuberculosis, lymphadenitis, skin disease, or disseminated disease. NTM include, but are not limited to, M. avium Complex (MAC), M. avium, M. kansasii, M. abscessus, M. chelonae, M. fortuitum, M. genavense, M. gordonae, M. haemophilum, M. immunogenum, M. intracellulare, M. malmoense, M. marinum, M. mucogenicum, M. nonchromogenicum, M. scrofulaceum, M. simiae, M. smegmatis, M. szulgai, M. terrae, M. terrae complex, M. ulcerans, and M. xenopi. MAC includes at least two mycobacterial species, M. avium and M. intracellulare. These two species cannot be differentiated on the basis of traditional physical or biochemical tests, but there are nucleic acid probes that can be used to identify and differentiate between the two species.

In certain embodiments, the acid-fast bacteria may be selected from one or more of M. smegmatis, M. tuberculosis, M. avium, M. kansasii, M. scrofulaceum, M. peregrinum, M. marinum, M. intracellulare, and/or M. fortuitum.

In some embodiments, infection with Gram-negative bacteria or acid-fast bacteria results in a localized infection, such as a topical bacterial infection, e.g., a skin wound. In other embodiments, the bacterial infection is a systemic pathogenic bacterial infection. Common acid-fast infections include tuberculosis and non-tuberculosis mycobacteria infections. Common Gram-negative pathogens and associated infections are listed in Table A of the present disclosure. These are meant to serve as examples of the bacterial infections that may be treated, mitigated or prevented with the present Chp peptides, lysins, or lysin-AMP constructs and active fragments thereof and are not intended to be limiting.

TABLE A
Medically relevant Gram-negative bacteria and associated diseases

| | |
|---|---|
| Salmonella typhimurium | Gastrointestinal (GI) infections - |
| | salmonellosis |
| Shigella spp. | shigellosis |
| Escherichia coli | Urinary tract infections (UTIs) |
| Acinetobacter baumanii | Wound infections |
| Pseudomonas aeruginosa | bloodstream infections and pneumonia |
| Klebsiella pneumoniae | UTIs, and bloodstream infections |
| Neisseria gonorrhoeae | Sexually transmitted diseases |
| | (STDs) - gonorrhea |
| Neisseria meningitides | Meningitis |
| Serratia spp. | Catheter contaminations, UTIs, |
| | and pneumonia |
| Proteus mirabilis | UTIs |
| Morganella spp. | UTIs |
| Providencia spp. | UTIs |
| Edwardsiella spp | UTIs |
| Salmonella typhi | GI infections - typhoid fever |
| Yersinia pestis | Bubonic and pneumonic plague |
| Yersinia enterocolitica | GI infections |
| Yersinia pseudotuberculosis | GI infections |
| Haemophilus influenza | Meningitis |
| Bartonella Quintana | Trench fever |
| Brucella spp. | Brucellosis |
| Bordetella pertussis | Respiratory - Whooping cough |
| Burkholderia spp. | Respiratory |
| Moraxella spp. | Respiratory |
| Francisella tularensis | Tularemia |
| Legionella pneumophila | Respiratory - Legionnaires' disease |
| Coxiella burnetiid | Q fever |
| Bacteroides spp. | Abdominal infections |
| Enterobacter spp. | UTIs and respiratory |
| Chlamydia spp. | STDs, respiratory, and ocular |
| Stenotrophomonas spp. | Medical device contaminations, UTIs, |
| | bloodstream infections, and pneumonia |

In some embodiments, the Clip peptides, lysins, or lysin-AMP constructs and active fragments thereof of the present disclosure are used to treat a subject at risk for acquiring an infection due to Gram-negative bacterium or acid-fast bacterium. Subjects at risk for acquiring a Gram-negative or acid-fast bacterial infection include, for example, cystic fibrosis patients, neutropenic patients, patients with necrotising enterocolitis, burn victims, patients with wound infections, and, more generally, patients in a hospital setting, in particular surgical patients and patients being treated using an implantable medical device such as a catheter, for example a central venous catheter, a Hickman device, or electrophysiologic cardiac devices, for example pacemakers and implantable defibrillators. Other patient groups at risk for infection with Gram-negative or acid-fast bacteria include without limitation patients with implanted prostheses such a total joint replacement (for example total knee or hip replacement).

In another aspect, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a Chp, peptide, lysin, or lysin-AMP construct or active fragment thereof as described herein, and a second effective amount of an antibiotic suitable for the treatment of Gram-negative bacterial infection. In certain aspects, the present disclosure is directed to a method of preventing or treating a bacterial infection comprising co-administering to a subject diagnosed with, at risk for, or exhibiting symptoms of abacterial infection, a combination of a first effective amount of the composition containing an effective amount of a Chp peptide, lysin, or lysin-AMP construct or active fragment thereof as described herein, and a second effective amount of an antibiotic suitable for the treatment of an acid-fast bacterial infection.

The Chp peptides, lysins, or lysin-AMP constructs and active fragments thereof of the present disclosure can be co-administered with standard care antibiotics or with antibiotics of last resort, individually or in various combinations as within the skill of the art. Traditional antibiotics used against mycobacterial infections include, for example, macrolides (clarithromycin, azithromycin), ethambutol, rifamycins (rifampin, rifabutin), isoniazid, pyrazinamide, and aminoglycosides (streptomycin, amikacin). Traditional antibiotics used against P. aeruginosa are described in Table B. Antibiotics for other Gram-negative bacteria, such as Klebsiella spp., Enterobacter spp., Escherichia coli, Citrobacter freundii, Salmonella typhimurium, Yersinia pestis, and Franciscella tulerensis, are similar to that provided in Table B for P. aeruginosa.

TABLE B
Antibiotics used for the treatment of Pseudomonas aeruginosa

| Class | Agent |
|---|---|
| Penicillins | Ticarcillin-clavulanate |
| | Piperacillin-tazobactam |
| Cephalosporins | Ceftazidime |
| | Cefepime |
| | Cefoperazone |
| | Ceftobiprole |
| | Cefazolin |
| Monobactams | Aztreonam |
| Fluoroquinolones | Ciprofloxacin |
| | Levofloxacin |
| Carbapenems | Imipenem |
| | Meropenem |
| | Doripenem |
| Aminoglycosides | Gentamicin |
| | Tobramycin |
| | Amikacin |
| | Netilmicin |
| Polymixins | Colistin |
| | Polymixin B |
| Phosphonic acids | Fosfomycin |

In more specific embodiments, the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B and colistin. In certain embodiments, the antibiotic is chosen from isoniazid, rifampin, ethambutol, and pyrazinamide.

Combining the Chp peptides, lysins, or lysin-AMP constructs or active fragments thereof of the present disclosure with antibiotics provides an efficacious antibacterial regimen. In some embodiments, co-administration of Chp peptides, lysins, or lysin-AMP constructs or active fragments thereof of the present disclosure with one or more antibiotics may be carried out at reduced doses and amounts of either the Chp peptides, lysins, or lysin-AMP constructs or active fragments thereof or the antibiotic or both, and/or reduced frequency and/or duration of treatment with augmented bactericidal and bacteriostatic activity, reduced risk of antibiotic resistance and with reduced risk of deleterious neurological or renal side effects (such as those associated with colistin or polymyxin B use). Prior studies have shown that total cumulative colistin dose is associated with kidney damage, suggesting that decrease in dosage or shortening of treatment duration using the combination therapy with Chp peptides, lysins, or lysin-AMP constructs or active fragments thereof could decrease the incidence of nephrotoxicity (Spapen et al. Ann Intensive Care. 1: 14 (2011), which is herein incorporated by reference in its entirety). As used herein the term “reduced dose” refers to the dose of one active ingredient in the combination compared to monotherapy with the same active ingredient. In some embodiments, the dose of Chp peptides, lysins, or lysin-AMP constructs or active fragments thereof or the antibiotic in a combination may be suboptimal or even subthreshold compared to the respective monotherapy. In certain embodiments, the dose of GN370 or active fragments thereof or meropenem in a combination may be suboptimal or even subthreshold compared to the respective monotherapy in treating a Gram-negative bacterial infection resulting from at least one of P. aeruginosa or B. cenocepacia.

In some embodiments, the present disclosure provides a method of augmenting antibiotic activity of one or more antibiotics against Gram-negative or acid-fast bacteria compared to the activity of said antibiotics used alone by administering to a subject the Chp peptides, lysins, or lysin-AMP constructs or active fragments thereof disclosed herein together with an antibiotic of interest. The combination is effective against the bacteria and permits resistance against the antibiotic to be overcome and/or the antibiotic to be employed at lower doses, decreasing undesirable side effects, such as the nephrotoxic and neurotoxic effects of polymyxin B.

In some embodiments, the present Chp peptides, lysins and lysin-AMP constructs are able to drive the suppression of antibiotic resistance in the Gram-negative bacteria, including MDR and/or XDR organisms, such as P. aeruginosa. Such suppression of antibiotic resistance may be determined by serial passage resistance experiments, as described herein. Suppression of the development of antibiotic resistance occurs when the antibiotic MIC value does not significantly increase, or increases to a lesser extent, when in the presence of a Chp peptide, lysin, or lysin-AMP construct, e.g., GN370. For example, suppression of the development of antibiotic resistance may be indicated when there is a 2-fold or less increase, such as no increase, in the MIC value after serial passage of the Gram-negative bacteria for 28 days in a serial passage resistance experiment. In certain embodiments, GN370 may suppress the development of antibiotic resistance in a Gram-negative pathogen, such as, for example P. aeruginosa. In certain embodiments, the Chp peptides, lysins and lysin-AMP constructs, such as GN370, may be present in a sub-MIC amount, such as ⅛^{th} of the MIC, 1/16^{th} of the MIC, or 1/32^{nd} of the MIC. In certain embodiments, the antibiotic whose resistance is suppressed by the Chp peptides, lysins and lysin-AMP constructs, such as GN370, is a fluoroquinone (such as levofloxacin), an aminoglycoside (such as tobramycin), or a carbapenem (such as meropenem).

The Chp peptides, lysins, or lysin-AMP constructs or active fragments thereof optionally in combination with antibiotics of the present disclosure can be further combined with additional permeabilizing agents of the outer membrane of the Gram-negative bacteria, including, but not limited to metal chelators, such as e.g. EDTA, TRIS, lactic acid, lactoferrin, polymyxins, citric acid (Vaara M. Microbial Rev. 56(3):395-441 (1992), which is herein incorporated by reference in its entirety).

In yet another aspect, the present disclosure is directed to a method of inhibiting the growth, or reducing the population, or killing of at least one species of Gram-negative bacteria or acid-fast bacteria, the method comprising contacting the bacteria with a composition containing an effective amount of a Chp peptide, lysin, or lysin-AMP construct or active fragment thereof as described herein, wherein the Chp peptide, lysin, or lysin-AMP construct or active fragment thereof inhibits the growth, or reduces the population, or kills at least one species of Gram-negative bacteria or acid-fast bacteria.

In some embodiments, inhibiting the growth, or reducing the population, or killing at least one species of Gram-negative bacteria or acid-fast bacteria comprises contacting bacteria with the Chp peptides, lysins, or lysin-AMP constructs or active fragments as described herein, wherein the bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the Chp peptides, lysins, or lysin-AMP constructs or active fragments thereof described herein include but are not limited to tubing and other surface medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-negative bacteria and/or acid-fast bacteria.

In further embodiments of the disclosure, Chp peptides, lysins, or lysin-AMP constructs or active fragments thereof described herein may inhibit the growth, or reduce the population, or kill at least one species of Gram-negative bacteria present in sputum, such as human sputum. In certain embodiments, the at least one species of Gram-negative bacteria is present in the sputum of a cystic fibrosis patient. In certain embodiments, the at least one species of Gram-negative bacteria present in the sputum is selected from at least one of P. aeruginosa, S. maltophilia, A. xylosoxidans, A. ruhlandii, A. dolens, B. cenocepacia, B. gladioli, B. multivorans, and P. apista.

### EXAMPLES

### Example 1: Amurin Peptide Activity Against Mycobacterium Species

The activity ofamurinpeptides againstMycobacterium species was tested inCAAT media, except M. tuberculosis, which was tested in H79 (ADC, T) media. MIC (Tg/mL) values were determined for 30 amurin peptides against seven different Mycobacterium species.

MIC values were determined using a modification of the standard broth microdilution reference method defined by the Clinical and Laboratory Standards Institute (CLSI), CLSL. 2015. Methods for Dilution Antimicrobial Susceptibility Tests for Bacteria That Grow Aerobically; Approved Standard-10th Edition. Clinical and Laboratory Standards Institute, Wayne, PA. The modification was based on the replacement of Mueller Hinton Broth with either CAA media (with and without NaCl) or CAA supplemented with 2.5% human serum. MIC is the minimum concentration of peptide sufficient to suppress at least 80% of the bacterial growth compared to control.

The results are shown in Table 4 below.

TABLE 4

| | M. | M. | M. | M. |
|---|---|---|---|---|
| | smegmatis | fortuitum | avium | kansasi |
| | (MC | (ATCC | (ATCC | (ATCC |
| Amurin | 155) | 6841) | 700898) | 12478) |
| AGT915 | 1 | 1 | >64 | 1 |
| ALCES1 | 2 | 4 | 8 | 2 |
| AVQ206 | 16 | 2 | 1 | 2 |
| Chp1 | 16 | 16 | 4 | 4 |
| Chp10 | 1 | 1 | 0.5 | 4 |
| Chp10-M1 (AM3) | 1 | 2 | 1 | 1 |
| Chp2 (AM1) | 4 | 2 | 1 | 2 |
| Chp2-M1 (AM2) | 0.5 | 4 | 1 | 2 |
| Chp4 | 4 | 4 | 1 | >0.125 |
| Chp4-M1 (AM16) | 0.25 | 2 | 1 | 1 |
| Chp6 (AM17) | 4 | 4 | 1 | 1 |
| Chp6-M1 (AM18) | 0.25 | 2 | 2 | 4 |
| Chp8 | 8 | 2 | 8 | 4 |
| Chp9 | 8 | 4 | >64 | 8 |
| Myo | >64 | >64 | 8 | >64 |
| Ecp-M1 | 1 | 16 | 1 | 1 |
| Ecp2 | 8 | 4 | 2 | 2 |
| Ecp3 | 8 | 2 | 1 | 2 |
| Ecp3-M1 | 1 | 8 | 0.5 | 2 |
| Fen7875 | 16 | 8 | 2 | 32 |
| Gkh1 | 4 | 1 | 4 | 2 |
| HH3930 | 16 | 8 | 4 | 32 |
| Spi1 | 16 | 8 | >64 | 0.5 |
| Spi2 | 4 | 4 | >64 | 1 |
| Spi2-M1 | 1 | 4 | >64 | 4 |
| Unp1 | 2 | 8 | 1 | 8 |
| Unp2 | 4 | 2 | 0.5 | 2 |
| Unp2-M1 (AM9) | 1 | 4 | 1 | 2 |
| Unp3-M1 | 8 | 4 | 8 | 16 |
| Unp4 | 16 | 4 | 1 | 4 |
| QE | >64 | >64 | >64 | >64 |
| Tobramycin | 0.125 | 4 | 0.125 | 16 |

| | M. | M. | M. |
|---|---|---|---|
| | scrofulaceum | intracellulare | tuberculosis |
| | (ATCC | (ATCC | (ATCC |
| Amurin | 19981) | 13950) | 35817) |
| AGT915 | >64 | >64 | 4 |
| ALCES1 | 2 | 2 | 8 |
| AVQ206 | 4 | 4 | 8 |
| Chp1 | 4 | 4 | 32 |
| Chp10 | 0.5 | 0.5 | 8 |
| Chp10-M1 (AM3) | 2 | 2 | 8 |
| Chp2 (AM1) | 0.5 | 0.5 | 8 |
| Chp2-M1 (AM2) | 16 | 0.5 | 8 |
| Chp4 | 2 | 1 | 16 |
| Chp4-M1 (AM16) | 1 | 1 | 8 |
| Chp6 (AM17) | 2 | 0.5 | 4 |
| Chp6-M1 (AM18) | 4 | 1 | 4 |
| Chp8 | 4 | 4 | 8 |
| Chp9 | 8 | 4 | 8 |
| Myo | NT | >64 | 16 |
| Ecp-M1 | 2 | 8 | 32 |
| Ecp2 | 4 | 8 | 4 |
| Ecp3 | 2 | 8 | 8 |
| Ecp3-M1 | 2 | 8 | 8 |
| Fen7875 | 16 | 8 | 4 |
| Gkh1 | 8 | 4 | 8 |
| HH3930 | 16 | 16 | 4 |
| Spi1 | 2 | 4 | 16 |
| Spi2 | 8 | >64 | 16 |
| Spi2-M1 | 16 | >64 | 8 |
| Unp1 | 8 | 32 | 4 |
| Unp2 | 4 | 1 | 8 |
| Unp2-M1 (AM9) | 4 | 2 | 4 |
| Unp3-M1 | 8 | >64 | 4 |
| Unp4 | 4 | 4 | 16 |
| QE | >64 | >64 | >64 |
| Tobramycin | 0.25 | 0.25 | 16 |

As indicated in bold in Table 4 above, the following seven peptides were identified as having the highest activity: Chp2 (AM1); Chp2-M1 (AM2); Chp10-M1 (AM3); Unp2-M1 (AM9); Chp4-M1 (AM16); Chp6 (AM17); and Chp6-M1 (AM18). These seven peptides, as well as one negative control (AM10), were then selected for further analysis. MIC determinations were made for the seven peptides against 5 clinical strains of MDR M. abscessus isolates, which were predicted to be clarithromycin-resistant (erm(41)), and 1 ATCC strain. MIC (g/mL) values were determined after 7 days of growth in AST media (CAAT), wherein clear endpoints were observed. The results are shown below in Table 5.

TABLE 5

| M. | | Chp2- | Chp10- | Unp2- | Chp4- | | Chp6- | |
|---|---|---|---|---|---|---|---|---|
| abscessus | Chp2 | M1 | M1 | M1 | M1 | Chp6 | M1 | Control |
| strain | (AM1) | (AM2) | (AM3) | (AM9) | (AM16) | (AM17) | (AM18) | (AM10) |
| BK55096 | 1 | 4 | 2 | 2 | 1 | 2 | 4 | >128 |
| BK51402 | 1 | 2 | 1 | 1 | 1 | 2 | 2 | >128 |
| BK55691 | 1 | 1 | 1 | 2 | 1 | 2 | 2 | >128 |
| BK51403 | 2 | 4 | 4 | 4 | 2 | 2 | 1 | >128 |
| (ATCC | | | | | | | | |
| 19977) | | | | | | | | |
| BK51408 | 4 | 8 | 4 | 4 | 4 | 4 | 4 | >128 |
| BK58156 | 2 | 1 | 1 | 2 | 2 | 4 | 16 | >128 |

As shown above, both Chp2 (AM1) and Chp10-M1 (AM3) were highly active against M. abscessus. Unp2-M1 (AM9), Chp4-M1 (AM16), and Chp6 (AM17) also showed good activity.

Next, MIC determinations were made for the seven amurin peptides against 8 clinical strains of M. tuberculosis isolates, including MDR forms. Testing was performed in 7H9 broth (ADC enriched+Tween80 0.002%), and plates were incubated for 20 days. The results are shown below in Table 6, wherein MIC values are indicated in g/mL, and antibiotic resistance for each strain is indicated in parentheses.

TABLE 6

| M. | | Chp2- | Chp10- | Unp2- | Chp4- | | Chp6- | |
|---|---|---|---|---|---|---|---|---|
| tuberculosis | Chp2 | M1 | M1 | M1 | M1 | Chp6 | M1 | Control |
| strain | (AM1) | (AM2) | (AM3) | (AM9) | (AM16) | (AM17) | (AM18) | (AM10) |
| TN13923_HD17 | 16 | 8 | 32 | 16 | 8 | 16 | 1 | >128 |
| (susc) | | | | | | | | |
| TN8109_LB | 4 | 2 | 16 | 16 | 8 | 8 | 1 | >128 |
| (MDR) | | | | | | | | |
| TN34503_001 | 0.5 | 8 | 16 | 16 | 16 | 16 | 0.5 | >128 |
| (susc) | | | | | | | | |
| TN12657_KQ15 | 16 | 4 | 16 | 16 | 8 | 8 | 1 | >128 |
| (IS) | | | | | | | | |
| TN8562_M33 | 4 | 2 | 16 | 16 | 8 | 4 | 2 | >128 |
| (susc) | | | | | | | | |
| TN18349_W738 | 0.5 | 2 | 4 | 8 | 4 | 1 | 2 | >128 |
| (susc) | | | | | | | | |
| TN10571_KY | 2 | 4 | 16 | 8 | 8 | 16 | 2 | >128 |
| (MDR) | | | | | | | | |
| TN10525_LL | 8 | 8 | 64 | 64 | 16 | 16 | 1 | >128 |
| (MDR) | | | | | | | | |

As shown above, Chp6-M1 (AM18) was the most active against the M. tuberculosis isolates tested, with an MIC range of 0.5-2 μg/mL. It is further noted that AM18, which is 40 amino acids in length, is identical to amino acid residues 5-44 of AM2.

### Example 2: Antimicrobial Activity of GN370 and Chp Peptides AM1, AM2, and AM3

Further to an analysis of 27,804 adult cystic fibrosis patients, the nine most prevalent Gram-negative pathogens were identified, and their prevalence shown below in Table 7. Salsgiver, E. L. et al., Changing Epidemiology of the Respiratory Bacteriology of Patients with Cystic Fibrosis, Chest 2016, 149(2):390-400.

TABLE 7
Gram-negative pathogens in cystic fibrosis patients

| Gram-negative pathogen | Overall % prevalence |
|---|---|
| P. aeruginosa | 74.13 |
| S. maltophilia | 10.97 |
| A. xylosoxidans | 7.5 |
| A. ruhlandii and A. dolens | (<1) |
| B. cenocepacia | 0.67 |
| B. gladioli and B. multivorans | 0.77 and 0.91 |
| P. apista | 0.17 |

Based on these nine most prevalent strains, direct lytic agents, including GN370, AM1, AM2, and AM3, were evaluated against various isolates from each of the 9 strains. MICs and MBECs (Example 3) were determined, and a sputum inhibition test was conducted, as described below in Example 5.

The MICs for were determined as described below using the broth microdilution method for meropenem, GN370, AM1, AM2, and AM3. The results are shown below in Tables 8-12.

TABLE 8
MICs of meropenem

| Organism (n) | MIC₅₀ | MIC₉₀ | Range |
|---|---|---|---|
| P. aeruginosa (30) | 4 | 32 | 0.06->32 |
| S. maltophilia (25) | >32 | >32 | 0.125->32 |
| A. xylosoxidans (30) | 4 | >32 | 0.25->32 |
| A. ruhlandii (30) | 16 | >32 | 2->32 |
| A. dolens (30) | 2 | >32 | 0.25->32 |
| B. cenocepacia (30) | 4 | 8 | 2-32 |
| B. gladioli (30) | 1 | 2 | 0.125->32 |
| B. multivorans (30) | 8 | 32 | 0.5->32 |
| P. apista (30) | >32 | >32 | >32 |

TABLE 9
MICs of GN370

| Organism (n) | MIC₅₀ | MIC₉₀ | Range |
|---|---|---|---|
| P. aeruginosa (20) | 0.25 | 0.5 | 0.06-1 |
| S. maltophilia (23) | 0.5 | 2 | 0.03-2 |
| A. xylosoxidans (30) | 8 | >32 | 0.0625->32 |
| A. ruhlandii (30) | 2 | >32 | 0.0625->32 |
| A. dolens (29) | 32 | >32 | 0.25->32 |
| B. cenocepacia (29) | >32 | >32 | 0.5->32 |
| B. gladioli (29) | >32 | >32 | 0.5->32 |
| B. multivorans (30) | 16 | >32 | 0.06->32 |
| P. apista (30) | >32 | >32 | 0.5->32 |

TABLE 10
MICs of AM1

| Organism (n) | MIC₅₀ | MIC₉₀ | Range |
|---|---|---|---|
| P. aeruginosa (30) | 0.25 | 0.5 | 0.016-1 |
| S. maltophilia (25) | 0.5 | 1 | 0.016-1 |
| A. xylosoxidans (30) | 0.5 | 2 | 0.016-4 |
| A. ruhlandii (30) | 1 | 4 | 0.125-8 |
| A. dolens (30) | 1 | 2 | 0.06-4 |
| B. cenocepacia (30) | >128 | >128 | 0.06->128 |
| B. gladioli (30) | 8 | 64 | 0.03-128 |
| B. multivorans (30) | 128 | >128 | 0.016->128 |
| P. apista (30) | 64 | >128 | 4->128 |

TABLE 11
MIC of AM2

| Organism (n) | MIC₅₀ | MIC₉₀ | Range |
|---|---|---|---|
| P. aeruginosa (30) | 0.06 | 0.25 | 0.016-0.5 |
| S. maltophilia (25) | 0.5 | 1 | 0.03-2 |
| A. xylosoxidans (30) | 2 | 64 | 0.016->128 |
| A. ruhlandii (30) | 2 | 16 | 0.016->128 |
| A. dolens (30) | 4 | 8 | 0.016-16 |
| B. cenocepacia (30) | >128 | >128 | 0.06->128 |
| B. gladioli (30) | 4 | 8 | 0.016-16 |
| B. multivorans (30) | >128 | >128 | 0.016->128 |
| P. apista (30) | >128 | >128 | 4->128 |

TABLE 12
MICs of AM3

| Organism (n) | MIC₅₀ | MIC₉₀ | Range |
|---|---|---|---|
| P. aeruginosa (30) | 0.06 | 0.25 | 0.016-1 |
| S. maltophilia (25) | 0.5 | 1 | 0.016-2 |
| A. xylosoxidans (30) | 2 | 16 | 0.016->128 |
| A. ruhlandii (30) | 1 | >128 | 0.0625->128 |
| A. dolens (30) | 2 | 8 | 0.016-16 |
| B. cenocepacia (30) | >128 | >128 | 0.016->128 |
| B. gladioli (30) | 32 | 128 | 0.016->128 |
| B. multivorans (30) | >128 | >128 | 0.016->128 |
| P. apista (30) | >128 | >128 | 32->128 |

As shown above, GN370 is highly active against all tested isolates of P. aeruginosa and S. maltophilia. AM1 was highly active against all tested isolates of P. aeruginosa, S. maltophilia, and Achromobacter spp. AM2 and AM3 were highly active against P. aeruginosa and S. maltophilia, and variably active against Achromobacter spp.

### Example 3: Antibiofilm Activity of GN370 and Chp Peptides AM1, AM2, and AM3

The antibiofilm activity of GN370, AM1, AM2, and AM3 was evaluated against various Gram-negative bacteria prevalent in cystic fibrosis patients, including P. aeruginosa, S. maltophilia, and A. xylosoxidans.

The MBEC (after 2-hour treatments) was determined as described in Jones and Wozniak, PsI Produced by Mucoid Pseudomonas aeruginosa Contributes to the Establishment of Biofilms and Immune Evasion, Am. Soc. Microbiol. 2017, 8(3):e00864-17. Meropenem and tobramycin were included as controls and were both poorly active, with MBEC₉₀ values of >256 μg/mL (data not shown).

The results are shown below in Tables 13-16 for GN370 (Table 13), AM1 (Table 14), AM2 (Table 15), and AM3 (Table 16).

TABLE 13
MBEC values for GN370

| Organism (n) | MBEC₅₀ | MBEC₉₀ | Range |
|---|---|---|---|
| P. aeruginosa (28) | 1 | 2 | 0.064-4 |
| S. maltophilia (26) | 1 | 2 | 0.064-4 |
| A. xylosoxidans (15) | 8 | >256 | 4->256 |
| A. ruhlandii (15) | 4 | 64 | 4-64 |
| A. dolens (15) | 32 | 128 | 4-128 |
| B. cenocepacia (15) | 256 | >256 | 4->256 |
| B. gladioli (15) | 256 | >256 | 4->256 |
| B. multivorans (15) | 8 | 64 | 4->256 |
| P. apista (15) | 32 | 64 | 8-64 |

TABLE 14
MBEC values for AM1

| Organism (n) | MBEC₅₀ | MBEC₉₀ | Range |
|---|---|---|---|
| P. aeruginosa (28) | 0.5 | 1 | 0.064-2 |
| S. maltophilia (26) | 1 | 2 | 0.25-2 |
| A. xylosoxidans (15) | 0.5 | 1 | 0.125-2 |
| A. ruhlandii (15) | 16 | 16 | 2-16 |
| A. dolens (15) | 2 | 4 | 1-8 |
| B. cenocepacia (15) | 16 | 32 | 2-32 |
| B. gladioli (15) | 4 | 16 | 0.5-32 |
| B. multivorans (15) | 2 | 4 | 2-4 |
| P. apista (15) | 8 | 16 | 4->32 |

TABLE 15
MBEC values for AM2

| Organism (n) | MBEC₅₀ | MBEC₉₀ | Range |
|---|---|---|---|
| P. aeruginosa (28) | 0.25 | 1 | 0.016-2 |
| S. maltophilia (26) | 1 | 2 | 0.125-4 |
| A. xylosoxidans (15) | 2 | 8 | 0.125-32 |
| A. ruhlandii (15) | 8 | 32 | 1-32 |
| A. dolens (15) | 4 | 8 | 1-8 |
| B. cenocepacia (15) | 16 | 32 | 2-32 |
| B. gladioli (15) | 4 | 4 | 1-32 |
| B. multivorans (15) | 2 | 16 | 1-16 |
| P. apista (15) | 8 | 16 | 8->32 |

TABLE 16
MBEC values for AM3

| Organism (n) | MBEC₅₀ | MBEC₉₀ | Range |
|---|---|---|---|
| P. aeruginosa (28) | 0.125 | 1 | 0.016-1 |
| S. maltophilia (26) | 1 | 2 | 0.125-16 |
| A. xylosoxidans (15) | 1 | 2 | 0.125-4 |
| A. ruhlandii (15) | 16 | 16 | 1-32 |
| A. dolens (15) | 2 | 16 | 1-32 |
| B. cenocepacia (15) | 16 | 32 | 1-32 |
| B. gladioli (15) | 4 | 16 | 1-32 |
| B. multivorans (15) | 2 | 4 | 1-8 |
| P. apista (15) | 8 | 8 | 4-16 |

As shown above, all of GN370, AM1, and AM2 were highly active against P. aeruginosa and S. maltophilia. AM1 was highly active against all strains tested for P. aeruginosa, S. maltophilia, A. xylosoxidans, and B. multivorans. AM2 was primarily active against P. aeruginosa and S. maltophilia, while AM3 was primarily active against P. aeruginosa and A. xylosoxidans.

Additionally, the antibiofilm activity of GN370 was determined for a range of different MDR and extensively drug resistant (XDR) isolates. As shown below in Table 17, GN370 demonstrated activity against the isolates with MBEC values of ≤4 μg/mL, similar to the MIC values reported below in Example 6.

TABLE 17
MBEC values for GN370 against MDR and XDR bacterial strains

| | MBEC (μg/mL) | | | | | | | |
|---|---|---|---|---|---|---|---|---|
| Organism (n) | 0.25 | 0.5 | 1 | 2 | 4 | MBEC₅₀ | MBEC₉₀ | Range |
| P. | — | — | 4 | 4 | 1 | 2 | 4 | 1-4 |
| aeruginosa | | | | | | | | |
| (9) | | | | | | | | |
| A. | — | 2 | 12 | 8 | — | 1 | 2 | 0.5-2 |
| baumannii | | | | | | | | |
| (22) | | | | | | | | |
| E. coli (11) | 2 | 5 | 2 | 2 | — | 0.5 | 2 | 0.25-2 |
| K. | — | 3 | 3 | 4 | 1 | 1 | 2 | 0.5-4 |
| pneumoniae | | | | | | | | |
| (11) | | | | | | | | |
| E. cloacae | — | 1 | 9 | 4 | — | 1 | 2 | 0.5-2 |
| (14) | | | | | | | | |

### Example 4: Broad Spectrum Synergy of GN370 with Antibiotics

The synergy of GN370 with meropenem was evaluated against 3 different strains of P. aeruginosa and three different strains of B. cenocepacia. The results are shown below in Table 18 and indicate that GN370 synergizes with meropenem against P. aeruginosa but not against B. cenocepacia.

TABLE 18
GN370 synergizes with meropenem

| | GN370 | GN370 | Meropenem | Meropenem | |
|---|---|---|---|---|---|
| Organism | (alone) | (dual agent) | (alone) | (dual agent) | FICₘᵢₙ |
| P. aeruginosa | 1 | 0.06 | 8 | 0.25 | 0.091 |
| (ATCC 27853, | | | | | (Synergy) |
| CFS 1766) | | | | | |
| P. aeruginosa | 2 | 0.06 | 32 | 4 | 0.155 |
| (CFS 2045) | | | | | (Synergy) |
| P. aeruginosa | 4 | 0.25 | 64 | 4 | 0.125 |
| (CFS 2061) | | | | | (Synergy) |
| B. cenocepacia | 128 | 64 | 32 | 32 | 1.5 |
| (CFS 2135) | | | | | (Indifferent) |
| B. cenocepacia | 256 | 128 | 64 | 64 | 1.5 |
| (CFS 2138) | | | | | (Indifferent) |
| B. cenocepacia | 256 | 128 | 64 | 32 | 1 |
| (CFS 2142) | | | | | (Additive) |

GN370 was further tested in combination with up to seven additional antibiotics (in addition to meropenem) against a variety of bacterial strains, including P. aeruginosa, A. baumannii, E. coli, E. cloacae, and K. pneumoniae. GN370 and the subject antibiotics were tested in a checkerboard assay format, and FICI values were determined, wherein ≤0.5 indicates synergy, >0.5-≤1 indicates additive; and >1 indicates indifferent. The results, shown below in Table 19, demonstrate that GN370 synergized with a broad range of antibiotics having multiple different methods of action.

TABLE 19
FICI values for GN370 with broad range of antibiotics

| | P. aeruginosa | A. baumannii | E. coli | E. cloacae | K. |
|---|---|---|---|---|---|
| | ATCC | ATCC | ATCC | ATCC | pneumoniae |
| | 27853 | BAA-747 | 25922 | 13047 | HM-44 |
| | (MIC = | (MIC = | (MIC = | (MIC = | (MIC = |
| Antibiotic | 1 μg/mL) | 1 μg/mL) | 0.5 μg/mL) | 2 μg/mL) | 4 μg/mL) |
| Amikacin | 0.375 | 0.625 | 0.25 | 0.75 | 0.375 |
| Aztreonam | 0.375 | 0.313 | 0.375 | 0.25 | 0.375 |
| Cefazolin | n.d. | 0.5 | n.d. | n.d. | 0.375 |
| Ciprofloxacin | n.d. | n.d. | 0.156 | 0.25 | 0.156 |
| Colistin | 0.499 | 0.375 | 0.375 | 0.25 | 0.187 |
| Gentamicin | 0..531 | 0.563 | 0.312 | 0.25 | 0.375 |
| Meropenem | 0.375 | 0.5 | 0.5 | 0.531 | 0.5 |
| Tobramycin | 0.5 | 0.5 | 0.25 | 0.5 | 0.375 |

### Example 5: Activity of Chp Peptides and Lysin-AMP Constructs in Sputum of Adult Cystic Fibrosis Patients

Sputum from 5 cystic fibrosis patients was pooled. The resulting mixture was not filtered, as it was very dense and not amenable to sterilization by filtration. Sputum is known to be a complex mixture of DNA, carbohydrates, and lipids (Salsgiver, 2016). As used herein, the term “sputum” refers to the mucus present in the respiratory tract of an animal, such as a human. Sputum may increase in thickness due to the irritation or infection, including bacterial infection, and in some instances, may be expelled by the animal while coughing. In certain instances, cystic fibrosis may be characterized by the presence of a thick, sticky mucus or sputum present in the lungs of cystic fibrosis patients.

The activity of GN370, AM1, AM2, AM3, and meropenem was determined in the sputum using a time-kill assay format as disclosed in Zhang et al., Antimicrobial peptide therapeutics for cystic fibrosis, Antimicrobial Agents Chemother. 2005; 49(7):2921-2927. The activity was tested using three representative organisms (P. aeruginosa, A. xylosoxidans, and S. maltophilia), which were spiked into the sputum at a concentration of 2×10⁵ CFU/mL. The sputum was then diluted to a final concentration of 10% for testing.

After adding the lytic agent (i.e., either GN370, AM1, AM2, or AM3) to the sputum mixture with bacteria, cultures were incubated at 37° C. for 24 hours and aliquots were removed at 1 hour, 3 hour, and 24 hour timepoints for quantitative plating. Untreated controls were included in addition to pretreatment plating (t=0) for a baseline CFU determination. Activity was also determined in the absence of sputum for comparison to activity observed in the presence of sputum.

The results for sputum incubated with P. aeruginosa are shown below in Tables 20 and 21.

TABLE 20
Lytic activity against P. aeruginosa in the presence of 10% sputum

Log10 CFU/mL of sputum

| | Sputum | | | |
|---|---|---|---|---|
| Lytic agent | control | 1 hr | 3 hr | 24 hr |
| GN370 | 5.3 | 3.9 | 3.6 | 2.3 |
| (MIC = 0.5) | | | | |
| AM1 | 5.3 | 2.3 | 2.3 | 2.3 |
| (MIC = 1) | | | | |
| AM2 | 5.3 | 2.3 | 2.3 | 2.3 |
| (MIC = 0.25) | | | | |
| AM3 | 5.3 | 2.3 | 2.3 | 2.3 |
| (MIC = 0.125) | | | | |
| Meropenem | 5.3 | 5.26 | 4.78 | 6.6 |
| (MIC = 0.125) | | | | |

TABLE 21
Lytic activity against P. aeruginosa in the absence of sputum

Log10 CFU/mL of sputum

| | Sputum | | | |
|---|---|---|---|---|
| Lytic agent | control | 1 hr | 3 hr | 24 hr |
| GN370 | 5.3 | 2.3 | 2.3 | 2.3 |
| (MIC = 0.5) | | | | |
| AM1 | 5.3 | 2.3 | 2.3 | 2.3 |
| (MIC = 1) | | | | |
| AM2 | 5.3 | 2.3 | 2.3 | 2.3 |
| (MIC = 0.25) | | | | |
| AM3 | 5.3 | 2.3 | 2.3 | 2.3 |
| (MIC = 0.125) | | | | |
| Meropenem | 5.3 | 5.38 | 4.98 | 6.38 |
| (MIC = 0.125) | | | | |

As demonstrated in Table 20 and 21 above, antibacterial activity of GN370, AM1, AM2, and AM3 against P. aeruginosa is not inhibited in the presence of sputum. At the 24 hour timepoint, there was no difference in activity for any of GN370, AM1, AM2, and AM3 in the presence or absence of sputum. GN370 showed a 1.5-log₁₀ reduction by 3 hours and complete killing by 24 hours (>3-log₁₀ reduction). All of AM1, AM2, and AM3 showed complete killing by 1 hour and maintained killing by 24 hours (i.e., no new growth observed). Meropenem, however, was poorly active both in the presence and absence of sputum.

The results for sputum incubated with S. maltophilia are shown below in Tables 22 and 23. As with P. aeruginosa, for S. maltophilia, none of GN370, AM1, AM2, and AM3 was inhibited in the presence of sputum.

TABLE 22
Lytic activity against S. maltophilia in the presence of 10% sputum

| | Log₁₀ CFU/mL of sputum | | | |
|---|---|---|---|---|
| Lytic agent | Sputum control | 1 hr | 3 hr | 24 hr |
| GN370 (MIC = 0.5) | 5.48 | 3.48 | 2.3 | 2.3 |
| AM1 (MIC = 0.5) | 5.48 | 2.3 | 2.3 | 2.3 |
| AM2 (MIC = 0.5) | 5.48 | 2.3 | 2.3 | 2.3 |
| AM3 (MIC = 0.25) | 5.48 | 2.3 | 2.3 | 2.3 |
| Meropenem (MIC = 32) | 5.48 | 3.78 | 4.26 | 6.3 |

TABLE 23
Lytic activity against S. maltophilia in the absence of sputum

| | Log10 CFU/mL of sputum | | | |
|---|---|---|---|---|
| Lytic agent | Sputum control | 1 hr | 3 hr | 24 hr |
| GN370 (MIC = 0.5) | 5.48 | 2.3 | 2.3 | 2.3 |
| AM1 (MIC = 0.5) | 5.48 | 2.3 | 2.3 | 2.3 |
| AM2 (MIC = 0.5) | 5.48 | 2.3 | 2.3 | 2.3 |
| AM3 (MIC = 0.25) | 5.48 | 2.3 | 2.3 | 2.3 |
| Meropenem (MIC = 32) | 5.48 | 5.04 | 4.7 | 7.95 |

After incubation with S. maltophilia, GN370 showed about a 2-log₁₀ reduction by 3 hours and complete killing by 24 hours. AM1, AM2, and AM3 showed complete killing by 1 hour and maintained killing at 24 hours. Meropenem was poorly active in the presence and absence of sputum.

Finally, the results for sputum incubated with A. xylosoxidans are shown below in Tables 24 and 25. As with P. aeruginosa and S. maltophilia, for A. xylosoxidans, none of GN370, AM1, AM2, and AM3 was inhibited in the presence of sputum.

TABLE 24
Lytic activity against A. xylosoxidans
in the presence of 10% sputum

| | Log₁₀ CFU/mL of sputum | | | |
|---|---|---|---|---|
| Lytic agent | Sputum control | 1 hr | 3 hr | 24 hr |
| GN370 (MIC = 0.25) | 5.42 | 3.6 | 3.3 | 2.3 |
| AM1 (MIC = 0.5) | 5.42 | 2.3 | 2.3 | 2.3 |
| AM2 (MIC = 1) | 5.42 | 2.3 | 2.3 | 2.3 |
| AM3 (MIC = 0.5) | 5.42 | 2.3 | 2.3 | 2.3 |
| Meropenem (MIC = 0.25) | 5.42 | 4.78 | 4.3 | 7.56 |

TABLE 25
Lytic activity against A. xylosoxidans in the absence of sputum

| | Log₁₀ CFU/mL of sputum | | | |
|---|---|---|---|---|
| Lytic agent | Sputum control | 1 hr | 3 hr | 24 hr |
| GN370 (MIC = 0.25) | 5.42 | 3.7 | 3.3 | 2.3 |
| AM1 (MIC = 0.5) | 5.42 | 2.3 | 2.3 | 2.3 |
| AM2 (MIC = 1) | 5.42 | 3.3 | 2.3 | 2.3 |
| AM3 (MIC = 0.5) | 5.42 | 4.3 | 3.3 | 2.3 |
| Meropenem (MIC = 0.25) | 5.42 | 4.9 | 5.3 | 7.3 |

After incubation with A. xylosoxidans, GN370 showed about a 2-log₁₀ reduction by 3 hours and complete killing by 24 hours (>3-log₁₀ reduction). AM1, AM2, and AM3 showed complete killing by 1 hour and maintained killing at 24 hours. Meropenem was poorly active in the presence and absence of sputum. Therefore, as shown above, all of the tested lytic agents exhibited a high level of activity in the presence of cystic fibrosis patient sputum.

### Example 6: CF-370 Exhibits Broad Spectrum Antimicrobial Activity

MIC values were determined by broth microdilution against clinical isolates and resistant isolates from a wide range of bacterial species, including isolates from the CDC Antibiotic Resistance bank, including the GN ESKAPE pathogens and E. coli, including carbapenem-resistant Enterobacteriaciae, Acinetobacter, and Pseudomonas. The results are shown in Table 26 below.

TABLE 26
GN370 Extended Spectrum Activity Range

| Organism | N | MIC₅₀ | MIC₉₀ | Range |
|---|---|---|---|---|
| Klebsiella pneumoniae | 41 | 2 | 4 | 0.125-4 |
| AR Bank | 32 | 1 | 4 | 0.125-4 |
| Weill Cornell | 9 | 2 | 4 | 0.25-4 |
| Acinetobacter baumannii | 67 | 1 | 1 | 0.0625-2 |
| AR Bank | 41 | 1 | 1 | 0.064-2 |
| Weill Cornell | 26 | 1 | 1 | 0.25-1 |
| Pseudomonas aeruginosa | 122 | 1 | 2 | 0.25-2 |
| AR Bank | 67 | 1 | 2 | 0.25-2 |
| Weill Cornell | 55 | 1 | 2 | 0.25-2 |
| Enterobacter cloacae | 37 | 1 | 4 | 0.016-8 |
| AR Bank | 7 | 1 | 4 | 0.25-8 |
| Weill Cornell | 30 | 2 | 4 | 0.25-8 |
| Escherichia coli | 30 | 0.25 | 1 | 0.0625-4 |
| AR Bank | 17 | 0.5 | 1 | 0.125-1 |
| Weill Cornell | 13 | 0.25 | 0.5 | 0.064-4 |
| Achromobacter xylosoxidans | 11 | >64 | >64 | 32->64 |
| Burkholderia cenocepacia | 15 | >64 | >64 | 32->64 |
| Burkholderia cepacia | 9 | >64 | >64 | >64 |
| Pandoraea apista | 11 | >64 | >64 | >64 |
| Stenotrophomonas maltophilia | 12 | 2 | 8 | 0.25-8 |
| Ralstonia mannitolilytica | 10 | >64 | >64 | >64 |
| Serratia marcescens | 9 | 32 | >64 | 4->64 |
| Citrobacter freundii | 2 | — | — | 0.5-2 |
| Enterobacter aerogenes | 1 | — | — | 1 |
| Klebsiella oxytoca | 1 | — | — | 4 |
| Kluyvera ascorbate | 1 | — | — | 1 |
| Raoultella ornithinolytica | 1 | — | — | 0.25 |
| Salmonella senftenberg | 1 | — | — | 2 |

As shown, GN370 exhibited MIC₉₀ values of 1 μg/mL for A. baumannii and E. coli, 2 μg/mL for P. aeruginosa, and 4 μg/mL for K. pneumoniae and E. cloacae. No cross-resistance was observed. The demonstration of bactericidal activity (defined as decreases of ≥3 log 10 CFU/mL in time-kill assays), synergy with antibiotics, antibiofilm activity in the MBEC assay, and the absence of resistance in serial passage resistance experiments, which were previously described for P. aeruginosa, was extended to the other GN ESKAPE pathogens and E. coli. It is noted that, as shown in Table 24, the activity of GN370 was tested against both AR Bank and Weill-Cornell isolates for K. pneumoniae, A. baumannii, P. aeruginosa, E. cloacae, and E. coli. A similar antibacterial activity for GN370 was observed for both sets of isolates.

Based on the MIC values as provided above in Table 24, GN370 demonstrates broad activity against clinical as well as MDR and XDR isolates of various bacterial species, including P. aeruginosa, K. pneumoniae, E. cloacae, A. baumannii and E. coli.

### Example 7—GN370 Time-Kill Assays Demonstrating Broad Spectrum Bactericidal Activity

A kinetic analysis of GN370 bactericidal activity was performed using a time-kill assay format in which CFU reductions of each target organism (P. aeruginosa, K. pneumoniae, A. baumannii, E. coli, and E. cloacae) were measured over 24 hours at 37° C. (at 1 hour, 3 hour, and 24 hour time points) in cultures treated with 5×, 2×, 1×, and 0.2×MIC levels of GN370. Bactericidal activity was defined as CFU reductions of ≥3-log 10 CFU/mL as compared to the inoculum at the 24-hour time point. See Methods for Determining Bactericidal Activity of Antimicrobial Agents; Approved Guideline, NCCLS document M26-A, CLSI 1999 (Wayne, PA). As shown below in Table 27, GN370 exhibited bactericidal activity at ≥1×MIC for each pathogen evaluated.

TABLE 27
CFU reduction of bacteria with GN370 in time-kill assay

| | Log10 CFU/mL | | | |
|---|---|---|---|---|
| Organism | 0 hr | 1 hr | 3 hr | 24 hr |
| K. pneumoniae ATCC 700603 | | | | |
| (MIC = 4 μg/mL) | | | | |
| 5x MIC | 5.3 | 1.5 | 1.5 | 1.5 |
| 2x MIC | 5.3 | 1.5 | 1.5 | 1.5 |
| 1x MIC | 5.3 | 2.0 | 1.5 | 1.5 |
| 0.2x MIC | 5.3 | 5.2 | 5.0 | 8.5 |
| Untreated | 5.3 | 4.5 | 5.1 | 8.6 |
| P. aeruginosa | | | | |
| PA25 (MIC = 2 μg/mL) | | | | |
| 5x MIC | 5.0 | 1.5 | 1.5 | 1.5 |
| 2x MIC | 5.0 | 1.5 | 1.5 | 1.5 |
| 1x MIC | 5.0 | 1.5 | 1.5 | 1.54 |
| 0.2x MIC | 5.0 | 4.5 | 4.7 | 9.5 |
| Untreated | 5.0 | 5.8 | 5.9 | 9.5 |
| A. baumannii ATCC BAA 747 | | | | |
| (MIC = 1 μg/mL) | | | | |
| 5x MIC | 5.2 | 1.5 | 1.5 | 1.5 |
| 2x MIC | 5.2 | 1.5 | 1.5 | 1.5 |
| 1x MIC | 5.2 | 1.5 | 1.5 | 1.5 |
| 0.2x MIC | 5.2 | 3.8 | 4.0 | 5.8 |
| Untreated | 5.2 | 5.0 | 5.7 | 8.7 |
| E. coli AR-128 | | | | |
| (MIC = 0.25 μg/mL) | | | | |
| 5x MIC | 5.0 | 3.0 | 1.5 | 1.5 |
| 2x MIC | 5.0 | 4.4 | 2.2 | 1.5 |
| 1x MIC | 5.0 | 4.4 | 2.5 | 1.5 |
| 0.2x MIC | 5.0 | 4.5 | 4.5 | 8.4 |
| Untreated | 5.0 | 4.8 | 5.5 | 8.4 |
| E. cloacae ATCC 29941 | | | | |
| (MIC = 2 μg/mL) | | | | |
| 5x MIC | 5.3 | 1.5 | 1.5 | 1.5 |
| 2x MIC | 5.3 | 1.6 | 1.6 | 1.6 |
| 1x MIC | 5.3 | 2.4 | 1.5 | 2.3 |
| 0.2x MIC | 5.3 | 5.0 | 5.0 | 9.0 |
| Untreated | 5.3 | 5.5 | 6.1 | 8.6 |

The data provided herein, including MIC and MBEC values, indicate that GN370 demonstrates broad and rapid activity against clinical as well as MDR and XDR isolates of various bacterial species, including P. aeruginosa, K. pneumoniae, E. cloacae, A. baumannii and E. coli and demonstrates synergy with a broad spectrum of antibiotics.

### Example 8—Serial Passage Resistance Development Study

A 2-fold dilution serial passage study was conducted with P. aeruginosa strain CFS 1292 against GN370, AM1, AM2, and AM3, as well as ciprofloxacin (in duplicate) over a 28-day time period. The MIC of subcultures was determined daily at days 0-28 and compared to an untreated control.

As of Day 28, no significant changes were observed in the MIC values for any of GN370, AM1, AM2, and AM3, demonstrating that none of GN370, AM1, AM2, or AM3 showed any propensity towards resistance. Similar results are reported, for example, in PCT Publication Nos. WO 2021/007107 and WO 2020/046747, incorporated by reference herein. In contrast, ciprofloxacin exhibited a 256-fold increase in MIC (1 μg/mL to 256 μg/mL). D'Lima et al. also found an increase in ciprofloxacin MIC during serial passage. See D'Lima et al., No Decrease in Susceptibility to NVC-422 in Multiple-Passage Studies with Methicillin-Resistant Staphylococcus aureus, S. aureus, Pseudomonas aeruginosa, and Escherichia coli, ANTIMICROBIAL AGENTS AND CHEMOTHERAPY 2012, 56(5): 2753-2755, which reports an increase of ciprofloxacin MIC of up to 32-fold over a 21 day serial passage.

Further serial passage studies were conducted with S. maltophilia and A. xylosoxidans in triplicate, using levofloxacin as a comparative antibiotic. For S. maltophilia, as of day 28, none of GN370, AM1, AM2, and AM3 showed any propensity towards resistance, as no increase in MIC was observed. In contrast, for levofloxacin, the MIC increased up to 256-fold, from 0.25 μg/mL to 64 μg/mL. For A. xylosoxidans, as of day 28, no increase in the MICs for AM1 was observed, but the levofloxacin MIC values increased 128-fold, from 0.25 μg/mL to 32 μg/mL, and, in another instance, 64-fold, from 2 μg/mL to 128 μg/mL.

Additionally, 2-fold dilution serial passage studies were conducted using ESKAPE pathogens P. aeruginosa, A. baumanii, E. coli, K. pneumoniae, and E. cloacae against GN370 over a 28-day time period. Two-fold dilution serial passage studies were further conducted using P. aeruginosa against ciprofloxacin and A. baumaii, E. coli, K. pneumoniae, and E. cloacae against levofloxacin, over a 28 days. Up to 3 distinct lineages were tested for each compound against each target organism, including P. aeruginosa (PA453), K. pneumoniae (1_1_5 HM-44), A. baumannii (ATCC BAA-747), E. cloacae (ATCC 13047), and E. coli (ATCC 25922). The MIC values for each organism are shown below in Table 36.

TABLE 36
MIC values (μg/mL) for Target Organisms

| | Isolate | | Levoflox- | |
|---|---|---|---|---|
| Organism | designation | GN370 | acin | Ciprofloxacin |
| K. pneumoniae | 1_1_5_HM-44 | 2 | 0.03125 | 0.03125 |
| A. baumannii | ATCC BAA-747 | 1 | 0.125 | 0.5 |
| P. aeruginosa | PA453 | 0.125 | 4 | 2 |
| E. cloacae | ATCC 13047 | 1 | 0.0625 | 0.03125 |
| E. coli | ATCC 25922 | 0.5 | 0.03125 | 0.03125 |

The MIC of subcultures was determined daily at days 0-28 and compared to an untreated control. No change in the MIC values for GN370 were observed for any of P. aeruginosa, A. baumanii, and E. coli. A 2-fold or less shift in GN370 MIC values was observed for K. pneumoniae and E. cloacae. In another instance, no change in MIC values for GN370 was observed for K. pneumoniae. For ciprofloxacin and levofloxacin, at least 40-fold MIC increases were observed in all cases, with a range of about 128-fold to 512-fold average MIC increases was observed. Specifically, against P. aeruginosa, ciprofloxacin MICs increased up to 512-fold in two independent passages. Levofloxacin MICS increased up to 512-fold for K. pneumoniae in three independent passages, 128-fold for A. baumannii in three independent passages, 256-fold for E. cloacae in three independent passages, and 128-fold for E. coli in three independent passages. Untreated control passages exhibited no change in MIC values over 28 days.

These results evidence a low propensity for spontaneous resistance in all of GN370, AM1, AM2, and AM3 in the tested bacterial strains, and a low propensity for resistance to the ESKAPE pathogens in GN370, in contrast to ciprofloxacin and levofloxacin.

### Example 9—Repeat Dose Study of AM1 in the Rabbit Pulmonary Infection Model

A rabbit pulmonary model was used to assess the efficacy of AM1 alone and in combination with an antibiotic (amikacin). Initially, rabbits were infected intratracheally with 6.8 log CFU of P. aeruginosa AR-769. Treatment commenced 6 hours post-infection (t=6). The treatment groups are shown below in Table 28, and all dosages were administered intravenously. The rabbits were sacrificed about 12 hours after the last dose of amikacin, and the bacterial burden (CFU/gram) in the lung was assessed.

TABLE 28
Treatment Groups in the Rabbit Pulmonary Model

| | | Dosage | Frequency of |
|---|---|---|---|
| Treatment | N | amount | Dosages |
| Vehicle control | 13 | N/A | N/A |
| Amikacin | 13 | 16 mg/kg | Every 8 hours |
| | | | (11 doses to t = 86 |
| | | | hours) |
| AM1 | 2 | 1 mg/kg | Every 24 hours |
| | | ( 1/10^{th} MTD) | (4 doses to 78 hours) |
| | 7 | 2.5 mg/kg | Every 24 hours |
| | | (¼^{th} MTD) | (4 doses to 78 hours) |
| | 8 | 5 mg/kg | Every 24 hours |
| | | (½ MTD) | (4 doses to 78 hours) |
| Amikacin + | 3 | 16 mg/kg | Every 8 hours |
| AM1 | | amikacin + 1 | (amikacin) + Every |
| | | mg/kg AM1 | 24 hours (AM1) |
| | 6 | 16 mg/kg | Every 8 hours |
| | | amikacin + 2.5 | (amikacin) + Every |
| | | mg/kg AM1 | 24 hours (AM1) |
| | 8 | 16 mg/kg | Every 8 hours |
| | | amikacin + 5 | (amikacin) + Every |
| | | mg/kg AM1 | 24 hours (AM1) |

Mean bacterial density in lungs from animals treated with amikacin or AM1 alone showed moderate to no CFU/g reduction, compared to the vehicle control group. Mean bacterial density in the lung tissue from animals treated with AM1 in addition to amikacin further decreased from 2.2 to 2.6 log CFU, as compared to amikacin alone. The results are shown below in Table 29.

TABLE 29
Bacterial Burden Reduction with AM1
alone or combined with amikacin

| | | Mean | Log CFU |
|---|---|---|---|
| | | Bacterial Density | change v. pre- |
| | | in Lung (Log | treatment |
| Treatment | Survival | CFU/lung) ± SD | (at 6 hours) |
| Pre-treatment | — | 7.47 ± 0.73 | — |
| control, t = 6 (n = 15) | | | |
| Vehicle control | 15% | 9.34 ± 1.10 | 1.83 |
| (n = 118) | | | |
| Amikacin (16 | 85% | 7.84 ± 1.06 | 0.35* |
| mg/kg) (n = 17) | | | |
| AM1 (1 mg/kg) | 0% | 9.02 ± 1.49 | 1.56 |
| (n = 7) | | | |
| AM1 (2.5 mg/kg) | 29% | 9.35 ± 0.84 | 1.97 |
| (n = 7) | | | |
| AM1 (5 mg/kg) | 37.5% | 8.98 ± 1.47 | 1.80 |
| (n = 8) | | | |
| Amikacin (16 | 100% | 6.61 ± 1.14 | −0.74* |
| mg/kg) + AM1 (1 | | | |
| mg/kg) (n = 8) | | | |
| Amikacin (16 | 100% | 5.61 ± 2.02 | −1.71** |
| mg/kg) + AM1 (2.5 | | | |
| mg/kg) (n = 6) | | | |
| Amikacin (16 | 100% | 5.55 ± 1.52 | −1.63** |
| mg/kg) + AM1 (5 | | | |
| mg/kg) (n = 8) | | | |

*p < 0.05 v. vehicle control
**p < 0.05 v. vehicle control, amikacin alone, or AM1 alone

This data evidences AM1's synergistic activity together with amikacin in reducing the bacterial burden in pulmonary tissues for P. aeruginosa.

### Example 10—GN370 and AM1 Activity Against Mucoid, α-Hemolytic, MDR, and Carbapenem-Resistant Isolates

The activity of GN370 and AM1 was evaluated against various bacterial isolates, including mucoid and α-hemolytic isolates of P. aeruginosa, MDR isolates, and carbapenem-resistant isolates. The MIC₅₀ and MIC₉₀ values (g/mL) for GN370 and AM1 were determined in the presence of these various isolates using the methods as described above in Example 2, and good activity was demonstrated for both GN370 and AM1 in all instances.

The MIC values for GN370 and AM1 were determined in the presence of mucoid P. aeruginosa isolates (n=11) and α-hemolytic P. aeruginosa isolates (n=13). The results are shown below in Table 30 and demonstrate that both GN370 and AM1 exhibit good activity against mucoid and α-hemolytic P. aeruginosa isolates.

TABLE 30
GN370 and AM1 Activity in Mucoid and α-Hemolytic Isolates

| | GN370 MIC₅₀ | GN370 MIC₉₀ | GN370 |
|---|---|---|---|
| Organism | (μg/mL) | (μg/mL) | MIC Range |
| P. aeruginosa | 0.25 | 1 | 0.0625-1 |
| (mucoid) (n = 11) | | | |
| P. aeruginosa (α- | 0.25 | 1 | 0.0625-1 |
| hemolytic) (n = 13) | | | |
| | AM1 MIC₅₀ | AM1 MIC₉₀ | AM1 |
| | (μg/mL) | (μg/mL) | MIC Range |
| P. aeruginosa | 0.25 | 0.5 | 0.016-1 |
| (mucoid) (n = 11) | | | |
| P. aeruginosa (α- | 0.25 | 0.5 | 0.016-1 |
| hemolytic) (n = 13) | | | |

The activity of GN370 and AM1 was further tested against various MDR isolates of P. aeruginosa, S. maltophilia, and A. xylosoxidans, wherein MDR indicates the bacterial isolate was not susceptible to at least one agent in three or more antibacterial categories. See Magiorakos, A. et al., Bacteria: an international expert proposal for interim standard definitions for acquired resistance, MICROBIOLOGY 2011; 18(3):268-281. The results are shown below in Table 31.

TABLE 31
GN370 and AM1 Activity Against MDR Isolates

| | GN370 MIC₅₀ | GN370 MIC₉₀ | GN370 |
|---|---|---|---|
| Organism | (μg/mL) | (μg/mL) | MIC Range |
| P. aeruginosa (n = 13) | 0.125 | 0.5 | 0.0625-0.5 |
| S. maltophilia (n = 8) | 0.25 | 0.5 | 0.0625-0.5 |
| | AM1 MIC₅₀ | AM1 MIC₉₀ | AM1 |
| | (μg/mL) | (μg/mL) | MIC Range |
| P. aeruginosa (n = 13) | 0.25 | 0.5 | 0.016-0.5 |
| S. maltophilia (n = 8) | 0.5 | 1 | 0.5-1 |
| A. xylosoxidans (n = 14) | 1 | 4 | 0.5-4 |

The activity of GN370 was additionally tested against various XDR isolates of S. maltophilia, wherein XDR indicates that the bacterial isolate was not susceptible to at least one agent in all but two or fewer antimicrobial categories. See Magiorakos, A. et al., Multidrug-resistant, extensively drug-resistant and pandrug-resistant bacteria: an international expert proposal for interim standard definitions for acquired resistance, CLIN MICROBIOL INFECT 2012; 18(3):268-281. GN370 MICs were determined by broth microdilution, and acquired resistance was determined by antibiogram using a total of 27 different antibiotics, using the criteria described in Magiorakos 2012. The results are shown below in Table 32.

TABLE 32
GN370 Activity Against XDR Isolates of S. maltophilia

| | GN370 MIC |
|---|---|
| Isolate No. | (μg/mL) |
| AU29729 | 1 |
| AU29873 | 0.5 |
| AU30243 | 1 |
| AU30793 | 1 |
| AU32079 | 0.03125 |
| AU33199 | 0.0625 |
| AU33860 | 2 |
| AU33921 | 2 |
| AU34723 | 2 |
| AU35419 | 2 |
| AU35661 | 0.0625 |
| AU36631 | 0.5 |
| AU36888 | 0.25 |
| AU37239 | 1 |
| AU37260 | 0.5 |
| AU37689 | 0.25 |
| AU37737 | 2 |

Based on the results, it is shown that the S. maltophilia isolates had a MIC₅₀ of 1 μg/mL, a MIC₉₀ of 21 μg/mL, and a range of 0.03125-2.

Finally, the activity of GN370 and AM1 was further tested against various carbapenem-resistant isolates of P. aeruginosa, S. maltophilia, and A. xylosoxidans. The results are shown below in Table 32 and show GN370's and AM1's strong activity against these carbapenem-resistance strains.

TABLE 32
GN370 and AM1 Activity Against Carbapenem-Resistant Isolates

| | GN370 MIC₅₀ | GN370 MIC₉₀ | GN370 |
|---|---|---|---|
| Organism | (μg/mL) | (μg/mL) | MIC Range |
| P. aeruginosa (n = 12) | 0.25 | 0.5 | 0.0625-1 |
| S. maltophilia (n = 21) | 0.5 | 2 | 0.03-2 |
| | AM1 MIC₅₀ | AM1 MIC₉₀ | AM1 |
| | (μg/mL) | (μg/mL) | MIC Range |
| P. aeruginosa (n = 17) | 0.25 | 0.5 | 0.016-0.5 |
| S. maltophilia (n = 23) | 0.5 | 1 | 0.016-1 |
| A. xylosoxidans (n = 7) | 0.5 | 1 | 0.06225-1 |

### Example 11—GN370 in the Rabbit Pneumonia Model

A rabbit pneumonia model was used to assess the in vivo efficacy of GN370 alone and in combination with an antibiotic (amikacin) for infection caused by an XDR strain of P. aeruginosa (AR-769). Unlike PA20, which is not XDR, the AR-769 strain of P. aeruginosa is resistant to meropenem (MIC=64 μg/mL) and only susceptible in vitro to amikacin (MIC=8 g/mL). The MIC for GN370 is 2 μg/mL.

Initially, pneumonia was induced in New Zealand White rabbits (female, 2.2-2.5 kg) by endotracheal inoculation with P. aeruginosa AR-769 (4×10⁹ CFU/animal). At 6 hours post-infection, rabbits were randomized and received treatment as shown in Table 33. The bacterial burden of the group receiving no therapy was evaluated after 6 hours to establish a baseline in target tissues pre-treatment. Treatment lasted 24 hours, and comparative efficacy was measured in four target tissues (each lung, kidneys, and spleen) via mean bacterial densities (log₁₀ CFU/g+/−SD) at 24 hours post-amikacin dosing.

TABLE 33
Treatment groups for rabbit pneumonia model

| | | Dosage | Frequency of |
|---|---|---|---|
| Treatment | N | amount | Dosages |
| No therapy | 10 | N/A | N/A |
| Vehicle control | 10 | N/A | N/A |
| Amikacin | 9 | 4 mg/kg | 3 doses every 8 hours, intravenously |
| GN370 | 10 | 3 mg/kg | Single dose, intravenously |
| | 10 | 10 mg/kg | Single dose, intravenously |
| Amikacin + | 10 | 4 mg/kg | Single dose GN370, intravenously; 3 |
| GN370 | | amikacin + | doses amikacin every 8 hours, |
| | | 3 mg/kg | intravenously |
| | | GN370 | |
| | 10 | 4 mg/kg | Single dose GN370, intravenously; 3 |
| | | amikacin + | doses amikacin every 8 hours, |
| | | 10 mg/kg | intravenously |
| | | GN370 | |

All animals (100%) treated with GN370 at 3 mg/kg and 10 mg/kg alone or in addition to amikacin survived until the end of the study, whereas 80% of vehicle-treated animals survived. The bacterial densities in both lungs, kidney tissues, and spleen tissues are shown in Table 34 below.

TABLE 34
Bacterial Burden Reduction (Log₁₀ CFU/g)

| | Lung #1 | Lung #2 | Kidneys | Spleen |
|---|---|---|---|---|
| Group | (Mean ± SD) | (Mean ± SD) | (Mean ± SD) | (Mean ± SD) |
| Pre-treatment control | 6.64 ± 1.35 | 6.98 ± 1.09 | 4.59 ± 0.92 | 4.38 ± 0.92 |
| Vehicle control | 7.81 ± 1.12 | 7.78 ± 0.91 | 6.04 ± 0.75 | 6.37 ± 0.77 |
| Amikacin (4 mg/kg) | 6.43 ± 1.47 | 6.63 ± 0.91 | 4.82 ± 0.82 | 4.63 ± 1.16 |
| GN370 (3 mg/kg) | 6.14 ± 0.99 | 5.60 ± 1.59 | 4.05 ± 0.91 | 3.84 ± 0.97 |
| GN370 (10 mg/kg) | 5.74 ± 1.62 | 5.57 ± 1.40 | 4.02 ± 0.90 | 3.94 ± 0.86 |
| GN370 (3 mg/kg) + | 4.99 ± 1.55 | 4.06 ± 1.05 | 3.51 ± 0.89 | 3.11 ± 0.57 |
| amikacin (4 mg/kg) | | | | |
| GN370 (10 mg/kg) + | 3.41 ± 1.33 | 3.25 ± 1.34 | 2.88 ± 0.94 | 2.74 ± 0.75 |
| amikacin (4 mg/kg) | | | | |

Treatment with amikacin alone at 4 mg/kg caused a 1.2 to 1.7 log₁₀ CFU/g reduction in bacterial densities in lungs, kidneys, and spleen compared to the vehicle treated controls. A single dose of GN370 administered alone at 3 mg/kg or 10 mg/kg significantly reduced bacterial densities by 1.9 to 2.5 log₁₀ CFU/g in lungs, kidneys, and spleen as compared to the vehicle control group. The mean bacterial densities in lungs from animals treated with amikacin-alone or GN370-alone decreased by 1.3 and ˜2.0-log₁₀ CFU/g, vs vehicle controls, respectively (p≤0.01222).

The addition of a single GN370 dose at 3 mg/kg or 10 mg/kg to the amikacin regimen significantly further reduced bacterial densities in all three tissues by 1.3 to 3.2 log₁₀ CFU/g compared with amikacin alone and 2.5 to 4.5 log₁₀ CFU/g as compared to vehicle control groups (p≤0.0054). A dose response relationship was observed when increasing dose of GN370 was administered in addition to amikacin.

Additionally, in vitro checkerboard assays for GN370 with and without amikacin against P. aeruginosa strain AR-769 were done according to methods described herein. The results are shown below in Table 35.

TABLE 35
In Vitro Checkerboard Assay Results for GN370

| | | GN370 | | | Antibiotic | | | | |
|---|---|---|---|---|---|---|---|---|---|
| | | MIC | | | MIC | | | | |
| | | (single | MIC | | (single | MIC | | | |
| Antibiotic | # | agent) | (combo) | FIC | agent) | (combo) | FIC | FICₘᵢₙ | FICₐᵥₑ |
| Amikacin | 1 | 2 | 0.063 | 0.031 | 0.5 | 0.125 | 0.25 | 0.281 | 0.323 |
| | 2 | 2 | 0.063 | 0.031 | 0.5 | 0.125 | 0.25 | 0.281 | 0.323 |
| Colistin | 1 | 2 | 0.25 | 0.125 | 0.063 | 0.008 | 0.125 | 0.25 | 0.292 |
| | 2 | 2 | 0.25 | 0.125 | 0.063 | 0.008 | 0.125 | 0.25 | 0.292 |
| Tobramycin | 1 | 2 | 0.5 | 0.25 | 0.125 | 0.016 | 0.125 | 0.375 | 0.418 |
| | 2 | 2 | 0.5 | 0.25 | 0.125 | 0.015 | 0.125 | 0.375 | 0.418 |
| Ceftazidime | 1 | 2 | 0.063 | 0.031 | 512 | 512 | 1 | 1.031 | 1.073 |
| | 2 | 2 | 0.063 | 0.031 | 512 | 512 | 1 | 1.031 | 1.073 |
| Cefepime | 1 | 2 | 0.5 | 0.25 | 256 | 64 | 0.25 | 0.5 | 0.542 |
| | 2 | 2 | 0.5 | 0.25 | 256 | 32 | 0.125 | 0.375 | 0.419 |
| Ciprofloxacin | 1 | 2 | 0.5 | 0.25 | 8 | 2 | 0.25 | 0.5 | 0.543 |
| | 2 | 2 | 0.5 | 0.25 | 8 | 1 | 0.125 | 0.375 | 0.417 |
| Meropenem | 1 | 2 | 0.5 | 0.25 | 64 | 8 | 0.125 | 0.375 | 0.422 |
| | 2 | 2 | 0.5 | 0.25 | 64 | 8 | 0.125 | 0.375 | 0.419 |

As shown above, the average FICI score for GN370 and amikacin was 0.323, and GN370 also exhibited synergy (average FICI score ≤0.5) with all of the antibiotics tested with the exception of ceftazidime.

These studies demonstrate in vitro synergy and in vivo efficacy of an intravenously-administered lysin targeting an XDR strain of P. aeruginosa. GN370 synergized with amikacin to improve efficacy in a pseudomonal pneumonia model, featuring hematogenous secondary infection in kidneys and spleen. Overall, these data support a potential role for GN370 adjunctive therapy against serious P. aeruginosa infections, including those caused by multi-antibiotic-resistant strains (MDR and XDR); hospital-acquired and/or ventilator-associated pneumonia; and cystic fibrosis-associated pneumonia.

### Example 12—GN370 Suppresses In Vitro Resistance to Antibiotics in P. aeruginosa

The in vitro serial (daily) passage resistance assay format described above was used for GN370 (MIC=1) and each of three antibiotics: levofloxacin (MIC=1), meropenem (MIC=0.5), and tobramycin (MIC=0.002). Up to three different lineages were tested for each condition against P. aeruginosa strain ATCC 27853. In single agent control studies, GN370 and antibiotic exposures followed a standard 2-fold dilution scheme. For combinations of GN370 and an antibiotic, a 2-fold dilution series of each antibiotic was used in addition to a constant sub-MIC level of GN370 (⅛^{th} MIC, or 0.125 μg/mL). For levofloxacin, additional sub-MIC amounts were evaluated, including 1/16^{th} MIC (0.0625 μg/mL) and 1/32^{nd} MIC (0.03125 μg/mL). On a daily basis for 28 days, all intermediaries were isolated and subcultured twice (in the absence of antimicrobial agent) prior to final MIC determinations.

When evaluated alone in the absence of GN370, after 28 days, a 16-fold MIC increase was observed for levofloxacin, while a 32-fold MIC increase was observed for meropenem, and a 32-fold MIC increase was observed for tobramycin. In another instance, up to a 4-fold MIC increase was observed for tobramycin in the absence of GN370. No MIC increases were observed for GN370 alone. In the presence of ⅛^{th} MIC of GN370, no change in MIC was observed for levofloxacin, meropenem, and tobramycin. In another instance, a 2-fold MIC increase was observed for meropenem in the presence of ⅛^{th} MIC of GN370. The use of both 1/16^{th} MIC and 1/32^{nd} MIC of GN370 resulted in an 2-fold increase in MIC for levofloxacin after 28 days.

These data indicate that GN370 exhibits a suppressive effect on antibiotic resistance at concentrations as low as 1/32^{nd} MIC and support the use of GN370 in addition to conventional antibiotics for the treatment of infections caused by drug-resistant Gram-negative pathogens. Suppression of the development of antibiotic resistance occurs when the antibiotic MIC value does not significantly increase, or increases to a lesser extent, when in the presence of a Chp peptide, lysin, or lysin-AMP construct, e.g., GN370.

Additional data was generated for each meropenem, levofloxacin, and tobramycin in the Antimicrobial Susceptibility Testing (AST) Media Cation-Adjusted Mueller Hinton Broth (CAMHB). MIC increases of up to 1024-fold, 64-fold, and 32-fold were observed for each of meropenem, levofloxacin, and tobramycin, respectively, over the 28-day serial passage. Serial passage of GN370 for 28 days in DCAAT media did not result in any increase in MIC. These data indicate that the serial passage resistance studies are being performed in a manner consistent with that reported in the literature, as they are consistent with results previously reported. See, e.g., Palmer, K. et al., Genetic Basis for Daptomycin Resistance in Enterococci, Antimicrobial Agents Chemother. 2011, 55(7), doi: 10.1128/AAC.00207-11; Drago, L. et al., In vitro selection of resistance in Pseudomonas aeruginosa and Acinetobacter spp. by levofloxacin and ciprofloxacin alone and in combination with Beta-lactams and amikacin, J. Microbial Chemother 2005, 56(2):353-359; Zhao, L. et al., Development of in vitro resistance to fluoroquinolones in Pseudomonas aeruginosa, Antimicrobial Resistance Infection Control 2020, 9(124):1-8; and Schuch, R., Combination therapy with lysin CF-301 and antibiotic is superior to antibiotic alone for treating methicillin-resistant Staphylococcus aureus-induced murine bacteremia, J. Infect Dis. 2014, 209(9):1469-78.

What is claimed is:

1. A method of inhibiting the growth, reducing the population, or killing of at least one species of Gram-negative bacteria, the method comprising contacting the bacteria with a composition comprising an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity for a period of time sufficient to inhibit said growth, reduce said population, or kill said at least one species of Gram-negative bacteria.

2. The method of claim 1, wherein the Chp peptide comprises an amino acid sequence selected from the group consisting of SEQ ID NO. 2, SEQ ID NO. 81, and SEQ ID NO. 89.

3. The method of claim 1, wherein the lysin-AMP construct comprises the amino acid sequence of SEQ ID NO: 149.

4. The method according to any of the preceding claims, wherein the at least one species of Gram-negative bacteria is selected from the group consisting of Klebsiella pneumoniae, Acinetobacter baumannii, Pseudomonas aeruginosa, Enterobacter cloacae, Escherichia coli, Achromobacter xylosoxidans, Achromobacter ruhlandii, Achromobacter dolens, Bulkholderia multivorans, Burkholderia cenocepacia, Burkholderia cepacia, Pandoraea apista, Stenotrophomonas maltophilia, Ralstonia mannitolilytica, Serratia marcescens, Citrobacter freundii, Enterobacter aerogenes, Klebsiella oxytoca, Kluyvera ascorbate, Raoultella ornithinolytica, and Salmonella senftenberg.

5. The method according to any of the preceding claims, wherein the at least one species of Gram-negative bacteria is selected from the group consisting of Klebsiella pneumoniae, Acinetobacter baumannii, Pseudomonas aeruginosa, Enterobacter cloacae and Escherichia coli.

6. The method according to any of the preceding claims, wherein the at least one species of Gram-negative bacteria is selected from the group consisting of Pseudomonas aeruginosa, Achromobacter xylosoxidans, Achromobacter ruhlandii, Achromobacter dolens, and Stenotrophomonas maltophilia.

7. The method according to any of the preceding claims, wherein the at least one species of Gram-negative bacteria is selected from the group consisting of Pseudomonas aeruginosa and Stenotrophomonas maltophilia.

8. A method of inhibiting a Gram-negative bacteria present in sputum comprising contacting the sputum with a composition comprising an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity for a period of time sufficient to inhibit said growth, reduce said population, or kill said at least one species of Gram-negative bacteria.

9. The method of claim 8, wherein the Chp peptide comprises an amino acid sequence selected from the group consisting of SEQ ID NO. 2, SEQ ID NO. 81, and SEQ ID NO. 89.

10. The method of claim 8, wherein the lysin-AMP construct comprises the amino acid sequence of SEQ ID NO: 149.

11. The method of any one of claims 8-10, wherein the at least one species of Gram-negative bacteria is selected from the group consisting of Pseudomonas aeruginosa, Achromobacter xylosoxidans, and Stenotrophomonas maltophilia.

12. A method of preventing, disrupting or eradicating a Gram-negative bacterial biofilm comprising:
administering (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, in an amount effective to kill at least one species of Gram-negative bacteria in the biofilm to a subject in need thereof.

13. The method of claim 12, wherein the Chp peptide comprises an amino acid sequence selected from the group consisting of SEQ ID NO. 2, SEQ ID NO. 81, and SEQ ID NO. 89.

14. The method of claim 12, wherein the lysin-AMP construct comprises the amino acid sequence of SEQ ID NO: 149.

15. The method of any one of claims 12-14, wherein the at least one species of Gram-negative bacteria is selected from the group consisting of Klebsiella pneumoniae, Acinetobacter baumannii, Pseudomonas aeruginosa, Enterobacter cloacae, Escherichia coli, Achromobacter xylosoxidans, Bulkholderia multivorans, and Stenotrophomonas maltophilia.

16. The method of any one of claims 1-15, wherein the at least one species of Gram-negative bacteria further comprises Pseudomonas aeruginosa.

17. The method of any one of claims 1-16, wherein the bacterial infection is a topical or systemic bacterial infection.

18. A method of treating a bacterial infection caused by a Gram-negative bacteria, wherein the Gram-negative bacteria comprises Burkholderia cenocepacia and optionally one or more additional species of Gram-negative bacteria, which method comprises administering to a subject diagnosed with, at risk for, or exhibiting symptoms of a bacterial infection, a pharmaceutical composition containing an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, wherein the composition comprises at least one activity selected from inhibiting Burkholderia cenocepacia bacterial growth, reducing a Burkholderia cenocepacia bacterial population and/or killing Burkholderia cenocepacia.

19. The method of claim 18, further comprising administering to the subject an antibiotic suitable for the treatment of a Gram-negative bacterial infection.

20. The method of claim 19, wherein the antibiotic suitable for the treatment of a Gram-negative bacterial infection is selected from one or more of azithromycin, aztreonam, fosfomycin, a cephalosporin, ciprofloxacin, levofloxacin, an aminoglycoside, a carbapenem, a penicillin, rifampicin, polymyxin B, and colistin.

21. The method of claim 19 or 20, wherein the antibiotic is a carbapenem, aztreonam, ciprofloxacin, an aminoglycoside, colistin, or a cephalosporin.

22. The method of claim 20 or 21, wherein the aminoglycoside is gentamycin, tobramycin, or amikacin.

23. The method of claim 21 or 22, wherein the cephalosporin is ceftazidime, cefepime, cefaperazone, ceftobiprole, or cefazolin.

24. The method according to any of claims 18-23 wherein the lysin-AMP construct comprises the amino acid sequence of SEQ ID NO: 149.

25. The method according to any of claims 1-4, 6, 8-15 and 18-24, wherein the at least one species of Gram-negative bacteria is selected from mucoid and/or α-hemolytic isolates of P. aeruginosa, and/or multidrug-resistant (MDR) isolates and/or carbapenem-resistant isolates of P. aeruginosa, S. maltophilia, and/or A. xylosoxidans.

26. The method according to any of claims 1-4, 6, 8-15 and 18-24, wherein the at least one species of Gram-negative bacteria is selected from extensively drug-resistant (XDR) isolates of P. aeruginosa.

27. A method of suppressing the ability of a Gram-negative bacteria to develop resistance to an antibiotic, the method comprising administering to the Gram-negative bacteria
a. the antibiotic; and
b. (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, in an amount effective to suppress the Gram-negative bacteria's development of resistance to the antibiotic.

28. The method according to claim 27, wherein the lysin-AMP construct comprises the amino acid sequence of SEQ ID NO: 149.

29. The method according to claim 27 or 28, wherein the antibiotic is a fluoroquinolone, optionally levofloxacin; a carbapenem, optionally meropenem; or an aminoglycoside, optionally tobramycin.

30. The method according to claims 27-29, wherein the Chp peptide, lysin, or lysin-AMP construct is administered in a sub-MIC amount to the Gram-negative bacteria.

31. A method of treating a bacterial infection caused by a Gram-negative bacteria, which method comprises administering to a subject in need thereof, a pharmaceutical composition containing an effective amount of (i) a Chp peptide, lysin, or lysin-AMP construct comprising an amino acid sequence selected from the group consisting of SEQ ID NOs. 1-26, 54-67, 81-102, and 129-161 or active fragments thereof, or (ii) a Chp peptide, lysin, or lysin-AMP construct having at least 80% sequence identity with the amino acid sequence selected from the group consisting of SEQ ID NOs. 1-26, 54-67, 81-102, and 129-161, said Chp peptide, lysin, or lysin-AMP construct having lytic activity, wherein the composition comprises at least one activity selected from inhibiting, reducing and/or killing the growth of said Gram negative bacteria.

32. The method of claim 31, wherein the infection is in cystic fibrosis (CF) patients and/or patients associated with pulmonary exacerbation (PEx) and/or decline in lung function and mortality.

33. The method of claim 31, wherein the infection is non-CF bronchiectasis and/or potentially acute pneumonias.

34. The method according to any of claims 1-3 or 31, wherein the at least one species of Gram-negative bacteria is mycobacterium species, e.g., selected from M. smegmatis, M. fortuitum, M. avium, M. kansaii, M. scrofulaceum, M. intracellulare and M. tuberculosis.

35. The method according to any of claims 1-3, wherein the at least one species of Gram-negative bacteria is M. abscessus.

36. The method according to any of the preceding claims wherein the bacteria is multidrug or extensively drug-resistant.


SEQUENCE LISTING (161 sequences)

SEQ ID NO: 1 - protein, 36 aa, Chlamydia virus Chp1
MVRRRRLRRRISRRIFRRTVARVGRRRRSFRGGIRF

SEQ ID NO: 2 - protein, 44 aa, Unknown
  Description of Unknown Chlamydia phage 2 sequence
MRLKMARRRYRLPRRRSRRLFSRTALRMHPRNRLRRIMRGGIRF

SEQ ID NO: 3 - protein, 35 aa, Chlamydia virus CPAR39
MCKKVCKKCPKKGPKNAPKIGAFYERKTPRLKQST

SEQ ID NO: 4 - protein, 39 aa, Chlamydia phage 4
MARRYRLSRRRSRRLFSRTALRMHRRNRLRRIMRGGIRF

SEQ ID NO: 5 - protein, 39 aa, Artificial Sequence
  Description of Artificial Sequence Synthetic polypeptide
MAEQYELSQEQSEQLFSETALQMHEQNELQEIMQGGIEF

SEQ ID NO: 6 - protein, 40 aa, Unknown
  Description of Unknown Guinea pig Chlamydia phage sequence
MARRRYRLPRRRSRRLFSRTALRMHPRNRLRRIMRGGIRF

SEQ ID NO: 7 - protein, 38 aa, Chlamydia trachomatis
MKRRKMTRKGSKRLFTATADKTKSINTAPPPMRGGIRL

SEQ ID NO: 8 - protein, 39 aa, Chlamydia trachomatis
MSKKRSRMSRRRSKKLFSKTALRTKSVNTRPPMRGGFRF

SEQ ID NO: 9 - protein, 40 aa, Chlamydia trachomatis
MSLRRHKLSRKASKRIFRKGASRTKTLNTRATPMRGGFRI

SEQ ID NO: 10 - protein, 38 aa, Chlamydia trachomatis
MKRRKLSKKKSRKIFTRGAVNVKKRNLRARPMRGGFRI

SEQ ID NO: 11 - protein, 37 aa, Chlamydia trachomatis
MAKKMTKGKDRQVFRKTADRTKKLNVRPLLYRGGIRL

SEQ ID NO: 12 - protein, 39 aa, Chlamydia trachomatis
MAGKKMVSKGKDRQIFRKTADRTKKMNVRPLLYRGGIRL

SEQ ID NO: 13 - protein, 41 aa, Marine gokushovirus
MRRPRKMNYKKSKRMFSRTAARTHRKNSLRGSRPMRGGIRL

SEQ ID NO: 14 - protein, 34 aa, Unknown
  Description of Unknown Gokushovirinae Fen672_31 sequence
MSKKASRKSFTKGAVKVHKKNVPTRVPMRGGIRL

SEQ ID NO: 15 - protein, 35 aa, Unknown
  Description of Unknown uncultured bacterium sequence
MKMRKRTDKRVFTRTAAKSKKVNIAPKIFRGGIRL

SEQ ID NO: 16 - protein, 39 aa, Escherichia coli
MARSRRRMSKRSSRRSFRKYAKTHKRNFKARSMRGGIRL

SEQ ID NO: 17 - protein, 47 aa, Unknown
  Description of Unknown Cognatishimia maritima sequence
MESPNSRSQLGITLYLLSTIFPDACFRYRRELPYPLVIWGVATLCLQ

SEQ ID NO: 18 - protein, 39 aa, Escherichia coli
MARSRRRMSKRSSRRSFRKYAKSHKKNFKARSMRGGIRL

SEQ ID NO: 19 - protein, 37 aa, Oscillibacter sp.
MRKRMSKRVDKKVFRRTAASAKKINIDPKIYRGGIRL

SEQ ID NO: 20 - protein, 37 aa, Unknown
  Description of Unknown uncultured bacterium sequence
MRRRRLSRRTSRRFFRKGLKVRRRNLRARPMRGGFRI

SEQ ID NO: 21 - protein, 39 aa, Unknown
  Description of Unknown uncultured bacterium sequence
MARRKKMKGKRDKRVFKQTANKTKAINISPKNMRGGTRL

SEQ ID NO: 22 - protein, 53 aa, Marine gokushovirus
MLTVWSDTPTIKRRKDMYRKRMSRKKSKKVFAKTAMKVNKRNHVKPMRGGYRI

SEQ ID NO: 23 - protein, 39 aa, Marine gokushovirus
MMKYRKKMSAKSSRKQFTKGAMKVKGKNFTKPMRGGIRL

SEQ ID NO: 24 - protein, 38 aa, Marine gokushovirus
MRRYNVNKGKSAKKFRKQVSKTKVANLRSNPMRGGWRL

SEQ ID NO: 25 - protein, 28 aa, Spiroplasma virus SpV4
MAYRGFKTSRVVKHRVRRRWFNHRRRYR

SEQ ID NO: 26 - protein, 38 aa, Spiroplasma virus SpV4
MRRKVKNTKRHQWRLTHSARSIKRANIMPSNPRGGRRF

SEQ ID NO: 27 - DNA, 111 bp, Chlamydia virus Chp1
atggttcgtagaagacgtttgagaagaagaataagtagaagaatttttagaagaacagtagctagagttggtagaaggcgaaggtcttttcgtggtggtattagattttaa

SEQ ID NO: 28 - DNA, 135 bp, Unknown
  Description of Unknown Chlamydia phage 2 sequence
atgaggttaaaaatggcacgaagaagatacagacttccgcgacgtagaagtcgaagacttttttcaagaactgcattgaggatgcatccaagaaataggcttcgaagaattatgcgtggcggcattaggttctag

SEQ ID NO: 29 - DNA, 108 bp, Chlamydia virus CPAR39
ttgtgcaaaaaagtgtgcaaaaaatgcccaaaaaaagggccaaaaaatgcccccaaaatcggagcattttacgagagaaaaacacctagacttaaacagtctacttga

SEQ ID NO: 30 - DNA, 120 bp, Chlamydia phage 4
atggcacgaagatacagactttcgcgacgcagaagtcgacgacttttttcaagaactgcattaagaatgcatcgaagaaatagacttcgaagaattatgcgtggcggcattaggttttag

SEQ ID NO: 31 - DNA, 120 bp, Artificial Sequence
  Description of Artificial Sequence Synthetic polynucleotide
atggcggaacagtatgaactgagccaggaacagagcgaacagctgtttagcgaaaccgcgctgcagatgcatgaacagaacgaactgcaggaaattatgcagggcggcattgaattttaa

SEQ ID NO: 32 - DNA, 123 bp, Unknown
  Description of Unknown Guinea pig Chlamydia phage sequence
atggcacgaagaagatacagacttccgcgacgtagaagtcgaagacttttttcaagaactgcattaaggatgcatccaagaaataggcttcgaagaattatgcgtggcggcattaggttctag

SEQ ID NO: 33 - DNA, 117 bp, Chlamydia trachomatis
atgaaacgtagaaaaatgacaagaaaaggttctaagcgtctttttactgcaactgctgataaaactaaatctatcaatactgccccgccgccaatgcgtggcggtatccggttgtaa

SEQ ID NO: 34 - DNA, 120 bp, Chlamydia trachomatis
atgtctaaaaagcgttctcgcatgtctcgccgccgttctaagaagttgttctcgaaaacggctctccgcacgaagagtgtcaacacccgtccgcctatgcgcggagggttccggttctga

SEQ ID NO: 35 - DNA, 123 bp, Chlamydia trachomatis
atgtctcttcgtcgtcataagctttctcgtaaggcgtctaagcgtatttttcgtaaaggtgcatcacgcacgaagactttgaatactcgtgctacgcctatgcgcggcggtttccgtatttaa

SEQ ID NO: 36 - DNA, 117 bp, Chlamydia trachomatis
atgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 37 - DNA, 114 bp, Chlamydia trachomatis
atggctaaaaaaatgactaaaggcaaggatcgtcaggtttttcgtaaaaccgctgatcgtactaagaaactcaatgttagaccgttgttatatcgaggaggtatcagattatga

SEQ ID NO: 38 - DNA, 120 bp, Chlamydia trachomatis
atggcaggaaaaaaaatggtatcaaaaggaaaagatagacagattttccgaaaaactgctgatcgcactaaaaaaatgaatgtgcgcccgctattatatcgtggaggtattagattatga

SEQ ID NO: 39 - DNA, 126 bp, Marine gokushovirus
atgagaagaccaagaaaaatgaactataaaaaatcaaaaagaatgttttcacgcacagcagcgagaacacacagaaaaaactctctaagaggtagccgacctatgagaggcggaatacgtctttaa

SEQ ID NO: 40 - DNA, 105 bp, Unknown
  Description of Unknown Gokushovirinae Fen672_31 sequence
atgtcgaagaaggcgtcgaggaagagttttactaagggtgccgttaaggttcataagaaaaatgttcctactcgtgttcctatgcgtggcggtattaggctttag

SEQ ID NO: 41 - DNA, 108 bp, Unknown
  Description of Unknown uncultured bacterium sequence
atgaaaatgcgtaagcggacggacaagcgagtgtttacccgcaccgctgctaagtccaagaaagtgaacattgccccgaaaatttttagaggaggtatccgtctgtga

SEQ ID NO: 42 - DNA, 120 bp, Escherichia coli
atggctcgttctcgccgtcgtatgtccaagcgttcttcccgtcgttcgttccgtaagtacgcaaagacgcataaacgtaactttaaagcccgctctatgcgtggtggaattcgtctttga

SEQ ID NO: 43 - DNA, 144 bp, Unknown
  Description of Unknown Cognatishimia maritima sequence
atggaaagcccgaacagccgcagccagctgggcattaccctgtatctgctgagcaccatttttccggatgcgtgctttcgctatcgccgcgaactgccgtatccgctggtgatttggggcgtggcgaccctgtgcctgcagtaa

SEQ ID NO: 44 - DNA, 120 bp, Escherichia coli
atggctcgttcccgtagacgtatgtctaagcgttcttcccgccgttcgttccgcaagtatgcgaagtcgcataagaagaactttaaagcccgctcaatgcgtggcggtatccgtttataa

SEQ ID NO: 45 - DNA, 114 bp, Oscillibacter sp.
atgagaaagcgaatgtctaagcgtgttgacaagaaggtgttccgtcgtactgccgcatctgccaagaagattaacattgaccccaagatttaccgtggaggtattcgcctatga

SEQ ID NO: 46 - DNA, 114 bp, Unknown
  Description of Unknown uncultured bacterium sequence
atgagacgtcgtcgtctatcccgcagaacttcccgccgttttttccgtaaaggacttaaggttcgccgtcgtaacctccgcgcgagacccatgagaggcggattcagaatttga

SEQ ID NO: 47 - DNA, 120 bp, Unknown
  Description of Unknown uncultured bacterium sequence
atggcacgacgcaagaagatgaaaggcaagcgggataaacgggtgtttaagcagacagccaacaaaaccaaggctatcaacatcagcccaaaaaacatgagagggggtacgagactgtga

SEQ ID NO: 48 - DNA, 162 bp, Marine gokushovirus
atgttaactgtgtggagtgacacccctaccataaaaaggagaaaagacatgtatagaaagagaatgtcaagaaagaaaagtaaaaaggtttttgcaaaaaccgcaatgaaagtaaataaaagaaaccacgttaaacctatgcgtggtggatatagaatataa

SEQ ID NO: 49 - DNA, 120 bp, Marine gokushovirus
atgatgaagtacagaaaaaaaatgagcgctaaaagtagccgaaagcaatttacaaaaggcgccatgaaagtgaagggtaaaaacttcacaaaaccaatgcgcggaggcatccgtctatag

SEQ ID NO: 50 - DNA, 117 bp, Marine gokushovirus
atgcgacgttacaatgtaaataaaggtaaatctgctaagaagtttcgaaagcaggtaagtaagacgaaggttgcaaacctacgttctaatccaatgcgaggtggttggagactctaa

SEQ ID NO: 51 - DNA, 87 bp, Spiroplasma virus SpV4
atggcttatcgtggttttaaaacgagtcgtgttgtaaaacatagagtacgtagaagatggtttaatcatagaagacgttatagatag

SEQ ID NO: 52 - DNA, 114 bp, Spiroplasma virus SpV4
atgcgtcgtaaagttaaaaacaccaaacgtcaccagtggcgtctgacccactctgctcgttctatcaaacgtgctaacatcatgccgtctaacccgcgtggtggtcgtcgtttc

SEQ ID NO: 53 - DNA, 135 bp, Unknown
  Description of Unknown Chlamydia phage 3 sequence
atgaggttaaaaatggcacgaagaagatacagacttccgcgacgtagaagtcgaagacttttttcaagaactgcattaaggatgcatccaagaaataggcttcgaagaattatgcgtggcggcattaggttctag

SEQ ID NO: 54 - protein, 44 aa, Unknown; identical to SEQ ID NO: 2
  Description of Unknown Chlamydia phage 3 sequence

SEQ ID NO: 55 - protein, 39 aa, Escherichia sp.
MARSRRRMSKRSSRRSFRKYAKTHKKNFKARSMRGGIRL

SEQ ID NO: 56 - protein, 39 aa, Escherichia sp.; identical to SEQ ID NO: 18

SEQ ID NO: 57 - protein, 55 aa, Unknown
  Description of Unknown Pseudomonas phage PP7 sequence
MSSTLCRWAVKALRCTRVYKEFIWKPLVALSYVTLYLLSSVFLSQLSYPIGSWAV

SEQ ID NO: 58 - protein, 35 aa, Unknown
  Description of Unknown Acinetobacter phage AP205 sequence
MKKRTKALLPYAVFIILSFQLTLLTALFMYYHYTF

SEQ ID NO: 59 - protein, 38 aa, Alces alces faeces associated microvirus MP12 5423
MAKKIRNKARDRRIFTRTASRMHKANRTPRFMRGGIRL

SEQ ID NO: 60 - protein, 38 aa, Unknown
  Description of Unknown Gokushovirinae environmental samples sequence
MRRKKMSRGKSKKLFRRTAKRVHRKNLRARPMRGGIRM

SEQ ID NO: 61 - protein, 39 aa, Unknown
  Description of Unknown Gokushovirinae environmental samples sequence
MAKRHKIPQRASQHSFTRHAQKVHPKNVPRLPMRGGIRL

SEQ ID NO: 62 - protein, 37 aa, Unknown
  Description of Unknown uncultured bacterium sequence
MRKKMHKSLDKRVFNRTAKKSKKINVNPVVYRGGIRL

SEQ ID NO: 63 - protein, 38 aa, Marine gokushovirus; identical to SEQ ID NO: 24

SEQ ID NO: 64 - protein, 41 aa, Unknown
  Description of Unknown Richelia intracellularis HH01 sequence
MRPVKRSRVNKARSAGKFRKQVGKTKMANLRSNPMRGGWRL

SEQ ID NO: 65 - protein, 41 aa, Unknown
  Description of Unknown Gokushovirinae Fen7875_21 sequence
MKPLKRKPVQKARSAAKFRRNVSTVKAANMAVKPMRGGWRF

SEQ ID NO: 66 - protein, 44 aa, Mycobacterium phage BabyRay
MTKRDIEYRKALGLNPSEPLPKIVGAVTRHGATLKRPRVTALAR

SEQ ID NO: 67 - protein, 38 aa, Unknown
  Description of Unknown Bdellovibrio phage phiMH2K sequence
MKRKPMSRKASQKTFKKNTGVQRMNHLNPRAMRGGIRL

SEQ ID NO: 68 - DNA, 120 bp, Escherichia sp.
atggctcgttctcgtcgtcgtatgtctaaacgttcttctcgtcgttcttttcgtaaatatgctaaaactcataaaaaaaattttaaagctcgttctatgcgtggaggaattcgtttataa

SEQ ID NO: 69 - DNA, 117 bp, Escherichia sp.
atggcgcgcagccgccgccgcatgagcaaacgcagcagccgccgcagctttcgcaaatatgcgaaaagccataaaaaaaactttaaagcgcgcagcatgcgcggcggcattcgcctg

SEQ ID NO: 70 - DNA, 165 bp, Unknown
  Description of Unknown Pseudomonas phage PP7 sequence
atgtcttctaccctgtgccgttgggctgttaaagctctgcgttgcacccgtgtttacaaagaattcatctggaaaccgctggttgctctgtcttacgttaccctgtacctgctgtcttctgttttcctgtctcagctgtcttacccgatcggttcttgggctgtt

SEQ ID NO: 71 - DNA, 108 bp, Unknown
  Description of Unknown Acinetobacter phage AP205 sequence
atgaagaaaaggacaaaagccttgcttccctatgcggttttcatcatactcagctttcaactaacattgttgactgccttgtttatgtattaccattataccttttag

SEQ ID NO: 72 - DNA, 117 bp, Alces alces faeces associated microvirus MP12 5423
atggcaaagaaaattagaaacaaagcacgtgatagacgtatcttcacaagaacagcttcacgcatgcacaaggcaaaccgcacaccaagatttatgagaggcggtattaggttatga

SEQ ID NO: 73 - DNA, 114 bp, Unknown
  Description of Unknown Gokushovirinae environmental samples sequence
atgcgtcgtaaaaaaatgtctcgtggtaaatctaaaaaactgttccgtcgtaccgctaaacgtgttcaccgtaaaaacctgcgtgctcgtccgatgcgtggtggtatccgtatg

SEQ ID NO: 74 - DNA, 117 bp, Unknown
  Description of Unknown Gokushovirinae environmental samples sequence
atggctaaacgtcacaaaatcccgcagcgtgcttctcagcactctttcacccgtcacgctcagaaagttcacccgaaaaacgttccgcgtctgccgatgcgtggtggtatccgtctg

SEQ ID NO: 75 - DNA, 111 bp, Unknown
  Description of Unknown uncultured bacterium sequence
atgcgtaaaaaaatgcacaaatctctggacaaacgtgttttcaaccgtaccgctaaaaaatctaaaaaaatcaacgttaacccggttgtttaccgtggtggtatccgtctg

SEQ ID NO: 76 - DNA, 117 bp, Marine gokushovirus; identical to SEQ ID NO: 50

SEQ ID NO: 77 - DNA, 126 bp, Unknown
  Description of Unknown Richelia intracellularis HH01 sequence
atgcgtccagttaaaagatcaagagtaaataaggcccgatctgcaggcaagtttcgtaagcaggtcggtaaaacaaagatggcaaatctgcgtagtaatccgatgcgcggcggatggcggctgtga

SEQ ID NO: 78 - DNA, 126 bp, Unknown
  Description of Unknown Gokushovirinae Fen7875_21 sequence
atgaagccattgaagcgtaagccggttcagaaggcgcggtcagcagccaagttccgtcgaaatgtgtctaccgttaaggctgccaatatggcggtgaagccgatgcgcggcggttggcggttctga

SEQ ID NO: 79 - DNA, 135 bp, Mycobacterium phage BabyRay
atgaccaagagagacatcgagtaccggaaagctttggggctcaacccatctgagccgctcccgaagattgtgggtgccgtcacccgccacggggccactctgaaacgcccacgggtcaccgcactggcccgatag

SEQ ID NO: 80 - DNA, 117 bp, Unknown
  Description of Unknown Bdellovibrio phage phiMH2K sequence
atgaaaagaaaaccaatgagccgcaaggcctctcaaaaaaccttcaaaaagaacacaggcgttcaacgcatgaaccatctcaacccacgcgccatgcgtggtggcattagactataa

SEQ ID NO: 81 - protein, 44 aa, Artificial Sequence; identical to SEQ ID NO: 2
  Description of Artificial Sequence Synthetic polypeptide
  MOD_RES (2)..(2): D-amino acid
  MOD_RES (4)..(4): D-amino acid
  MOD_RES (7)..(9): D-amino acid
  MOD_RES (11)..(11): D-amino acid
  MOD_RES (14)..(16): D-amino acid
  MOD_RES (18)..(19): D-amino acid
  MOD_RES (23)..(23): D-amino acid
  MOD_RES (27)..(27): D-amino acid
  MOD_RES (31)..(31): D-amino acid
  MOD_RES (33)..(33): D-amino acid
  MOD_RES (35)..(36): D-amino acid
  MOD_RES (39)..(39): D-amino acid
  MOD_RES (43)..(43): D-amino acid

SEQ ID NO: 82 - protein, 45 aa, Artificial Sequence
  Description of Artificial Sequence Synthetic polypeptide
MRLKMARRRYRLPRRRSRRLFSRTALRMHPRNRLRRIMRGGIRFC

SEQ ID NO: 83 - protein, 54 aa, Artificial Sequence
  Description of Artificial Sequence Synthetic polypeptide
APKAMRLKMARRRYRLPRRRSRRLFSRTALRMHPRNRLRRIMRGGIRFLQKKGI

SEQ ID NO: 84 - protein, 60 aa, Artificial Sequence
  Description of Artificial Sequence Synthetic polypeptide
MARRYRLSRRRSRRLFSRTALRMHRRNRLRRIMRRLFSRTALRMHRRNRLRRIMRGGIRF

SEQ ID NO: 85 - protein, 44 aa, Artificial Sequence
  Description of Artificial Sequence Synthetic polypeptide
GRLYRFHRPRRRNAIGMSRMRRKMFLRRMLRLISRRTRRPRLRA

SEQ ID NO: 86 - protein, 39 aa, Artificial Sequence
  Description of Artificial Sequence Synthetic polypeptide
RTRNFRIRRAKARRKMMLSHFKYGMARKGSKSRSSRRSR

SEQ ID NO: 87 - protein, 39 aa, Artificial Sequence; identical to SEQ ID NO: 16
  Description of Artificial Sequence Synthetic polypeptide
  MOD_RES (3)..(3): D-amino acid
  MOD_RES (5)..(7): D-amino acid
  MOD_RES (10)..(11): D-amino acid
  MOD_RES (14)..(15): D-amino acid
  MOD_RES (18)..(19): D-amino acid
  MOD_RES (22)..(22): D-amino acid
  MOD_RES (25)..(26): D-amino acid
  MOD_RES (29)..(29): D-amino acid
  MOD_RES (31)..(31): D-amino acid
  MOD_RES (34)..(34): D-amino acid
  MOD_RES (38)..(38): D-amino acid

SEQ ID NO: 88 - protein, 40 aa, Artificial Sequence; identical to SEQ ID NO: 6
  Description of Artificial Sequence Synthetic polypeptide
  MOD_RES (3)..(5): D-amino acid
  MOD_RES (7)..(7): D-amino acid
  MOD_RES (10)..(12): D-amino acid
  MOD_RES (14)..(15): D-amino acid
  MOD_RES (19)..(19): D-amino acid
  MOD_RES (23)..(23): D-amino acid
  MOD_RES (27)..(27): D-amino acid
  MOD_RES (29)..(29): D-amino acid
  MOD_RES (31)..(32): D-amino acid
  MOD_RES (35)..(35): D-amino acid
  MOD_RES (39)..(39): D-amino acid

SEQ ID NO: 89 - protein, 38 aa, Artificial Sequence; identical to SEQ ID NO: 10
  Description of Artificial Sequence Synthetic polypeptide
  MOD_RES (2)..(5): D-amino acid
  MOD_RES (8)..(10): D-amino acid
  MOD_RES (12)..(13): D-amino acid
  MOD_RES (17)..(17): D-amino acid
  MOD_RES (23)..(25): D-amino acid
  MOD_RES (28)..(28): D-amino acid
  MOD_RES (30)..(30): D-amino acid
  MOD_RES (33)..(33): D-amino acid
  MOD_RES (37)..(37): D-amino acid

SEQ ID NO: 90 - protein, 38 aa, Artificial Sequence; identical to SEQ ID NO: 59
  Description of Artificial Sequence Synthetic polypeptide
  MOD_RES (3)..(4): D-amino acid
  MOD_RES (6)..(6): D-amino acid
  MOD_RES (8)..(8): D-amino acid
  MOD_RES (10)..(10): D-amino acid
  MOD_RES (12)..(13): D-amino acid
  MOD_RES (17)..(17): D-amino acid
  MOD_RES (21)..(21): D-amino acid
  MOD_RES (24)..(24): D-amino acid
  MOD_RES (27)..(27): D-amino acid
  MOD_RES (30)..(30): D-amino acid
  MOD_RES (33)..(33): D-amino acid
  MOD_RES (37)..(37): D-amino acid

SEQ ID NO: 91 - protein, 39 aa, Artificial Sequence; identical to SEQ ID NO: 4
  Description of Artificial Sequence Synthetic polypeptide
  MOD_RES (3)..(4): D-amino acid
  MOD_RES (6)..(6): D-amino acid
  MOD_RES (9)..(11): D-amino acid
  MOD_RES (13)..(14): D-amino acid
  MOD_RES (18)..(18): D-amino acid
  MOD_RES (22)..(22): D-amino acid
  MOD_RES (25)..(26): D-amino acid
  MOD_RES (28)..(28): D-amino acid
  MOD_RES (30)..(31): D-amino acid
  MOD_RES (34)..(34): D-amino acid
  MOD_RES (38)..(38): D-amino acid

SEQ ID NO: 92 - protein, 44 aa, Artificial Sequence
  Description of Artificial Sequence Synthetic polypeptide
MRLRYGHRRMTAGRIRMRSRRKFMLPRFRLLRIPRRSNRRRLRA

SEQ ID NO: 93 - protein, 44 aa, Artificial Sequence; identical to SEQ ID NO: 92
  Description of Artificial Sequence Synthetic polypeptide
  MOD_RES (2)..(2): D-amino acid
  MOD_RES (4)..(4): D-amino acid
  MOD_RES (8)..(9): D-amino acid
  MOD_RES (14)..(14): D-amino acid
  MOD_RES (16)..(16): D-amino acid
  MOD_RES (18)..(18): D-amino acid
  MOD_RES (20)..(22): D-amino acid
  MOD_RES (27)..(27): D-amino acid
  MOD_RES (29)..(29): D-amino acid
  MOD_RES (32)..(32): D-amino acid
  MOD_RES (35)..(36): D-amino acid
  MOD_RES (39)..(41): D-amino acid
  MOD_RES (43)..(43): D-amino acid

SEQ ID NO: 94 - protein, 40 aa, Marine gokushovirus
MIVRRHKMSRRRSRKLFSKTASRTRSKNLRSRPMRGGYRI

SEQ ID NO: 95 - protein, 38 aa, Artificial Sequence; identical to SEQ ID NO: 7
  Description of Artificial Sequence Synthetic polypeptide
  MOD_RES (3)..(5): D-amino acid
  MOD_RES (8)..(9): D-amino acid
  MOD_RES (12)..(13): D-amino acid
  MOD_RES (21)..(21): D-amino acid
  MOD_RES (23)..(23): D-amino acid
  MOD_RES (33)..(33): D-amino acid
  MOD_RES (37)..(37): D-amino acid

SEQ ID NO: 96 - protein, 37 aa, Artificial Sequence; identical to SEQ ID NO: 19
  Description of Artificial Sequence Synthetic polypeptide
  MOD_RES (2)..(4): D-amino acid
  MOD_RES (7)..(8): D-amino acid
  MOD_RES (11)..(12): D-amino acid
  MOD_RES (15)..(16): D-amino acid
  MOD_RES (22)..(23): D-amino acid
  MOD_RES (29)..(29): D-amino acid
  MOD_RES (32)..(32): D-amino acid
  MOD_RES (36)..(36): D-amino acid

SEQ ID NO: 97 - protein, 37 aa, Artificial Sequence; identical to SEQ ID NO: 20
  Description of Artificial Sequence Synthetic polypeptide
  MOD_RES (2)..(5): D-amino acid
  MOD_RES (8)..(9): D-amino acid
  MOD_RES (12)..(13): D-amino acid
  MOD_RES (16)..(17): D-amino acid
  MOD_RES (20)..(20): D-amino acid
  MOD_RES (22)..(24): D-amino acid
  MOD_RES (27)..(27): D-amino acid
  MOD_RES (29)..(29): D-amino acid
  MOD_RES (32)..(32): D-amino acid
  MOD_RES (36)..(36): D-amino acid

SEQ ID NO: 98 - protein, 39 aa, Artificial Sequence; identical to SEQ ID NO: 21
  Description of Artificial Sequence Synthetic polypeptide
  MOD_RES (3)..(6): D-amino acid
  MOD_RES (8)..(8): D-amino acid
  MOD_RES (10)..(11): D-amino acid
  MOD_RES (13)..(14): D-amino acid
  MOD_RES (17)..(17): D-amino acid
  MOD_RES (22)..(22): D-amino acid
  MOD_RES (24)..(24): D-amino acid
  MOD_RES (31)..(31): D-amino acid
  MOD_RES (34)..(34): D-amino acid
  MOD_RES (38)..(38): D-amino acid

SEQ ID NO: 99 - protein, 37 aa, Artificial Sequence
  Description of Artificial Sequence Synthetic polypeptide
  MOD_RES (2)..(4): D-amino acid
  MOD_RES (6)..(6): D-amino acid
  MOD_RES (9)..(10): D-amino acid
  MOD_RES (14)..(14): D-amino acid
  MOD_RES (20)..(20): D-amino acid
  MOD_RES (23)..(24): D-amino acid
  MOD_RES (33)..(33): D-amino acid
  MOD_RES (36)..(37): D-amino acid
MRRKVKNTKRHQWRLTHSARSIKRANIMPSNPRGGRR

SEQ ID NO: 100 - protein, 39 aa, Artificial Sequence; identical to SEQ ID NO: 55
  Description of Artificial Sequence Synthetic polypeptide
  MOD_RES (3)..(3): D-amino acid
  MOD_RES (5)..(7): D-amino acid
  MOD_RES (10)..(11): D-amino acid
  MOD_RES (14)..(15): D-amino acid
  MOD_RES (18)..(19): D-amino acid
  MOD_RES (22)..(22): D-amino acid
  MOD_RES (25)..(26): D-amino acid
  MOD_RES (29)..(29): D-amino acid
  MOD_RES (31)..(31): D-amino acid
  MOD_RES (34)..(34): D-amino acid
  MOD_RES (38)..(38): D-amino acid

SEQ ID NO: 101 - protein, 38 aa, Artificial Sequence; identical to SEQ ID NO: 24
  Description of Artificial Sequence Synthetic polypeptide
  MOD_RES (2)..(3): D-amino acid
  MOD_RES (8)..(8): D-amino acid
  MOD_RES (10)..(10): D-amino acid
  MOD_RES (13)..(14): D-amino acid
  MOD_RES (16)..(17): D-amino acid
  MOD_RES (21)..(21): D-amino acid
  MOD_RES (23)..(23): D-amino acid
  MOD_RES (28)..(28): D-amino acid
  MOD_RES (33)..(33): D-amino acid
  MOD_RES (37)..(37): D-amino acid

SEQ ID NO: 102 - protein, 46 aa, Mycobacterium virus Peaches
MKLTKSDIAYREALGLSTTDPLPAEIGMVTRRANRLKRPRKTARFR

SEQ ID NO: 103 - DNA, 120 bp, Marine gokushovirus
atgatcgttcgtcgtcacaaaatgtctcgtcgtcgttctcgtaaactgttctctaaaaccgcttctcgtacccgttctaaaaacctgcgttctcgtccgatgcgtggtggttaccgtatc

SEQ ID NO: 104 - DNA, 138 bp, Mycobacterium virus Peaches
atgaaactgaccaaatctgacatcgcttaccgtgaagctctgggtctgtctaccaccgacccgctgccggctgaaatcggtatggttacccgtcgtgctaaccgtctgaaacgtccgcgtaaaaccgctcgtttccgt

SEQ ID NO: 105 - DNA, 132 bp, Artificial Sequence
  Description of Artificial Sequence Synthetic polynucleotide
atgaggttaaaaatggcacgaagaagatacagacttccgcgacgtagaagtcgaagacttttttcaagaactgcattgaggatgcatccaagaaataggcttcgaagaattatgcgtggcggcattaggttc

SEQ ID NO: 106 - DNA, 135 bp, Artificial Sequence
  Description of Artificial Sequence Synthetic polynucleotide
atgaggttaaaaatggcacgaagaagatacagacttccgcgacgtagaagtcgaagacttttttcaagaactgcattgaggatgcatccaagaaataggcttcgaagaattatgcgtggcggcattaggttctgt

SEQ ID NO: 107 - DNA, 159 bp, Artificial Sequence
  Description of Artificial Sequence Synthetic polynucleotide
gctcctaaagctaggttaaaaatggcacgaagaagatacagacttccgcgacgtagaagtcgaagacttttttcaagaactgcattgaggatgcatccaagaaataggcttcgaagaattatgcgtggcggcattaggttcttacaaaaaaaaggaatt

SEQ ID NO: 108 - DNA, 180 bp, Artificial Sequence
  Description of Artificial Sequence Synthetic polynucleotide
atggctcgtcgttatcgtttatctcgtcgtcgttctcgtcgtttattttctcgtactgctttacgtatgcatcgtcgtaatcgtttacgtcgtattatgcgtcgtttattttctcgtactgctttacgtatgcatcgtcgtaatcgtttacgtcgtattatgcgtggaggaattcgtttt

SEQ ID NO: 109 - DNA, 132 bp, Artificial Sequence
  Description of Artificial Sequence Synthetic polynucleotide
ggacgtttatatcgttttcatcgtcctcgtcgtcgtaatgctattggaatgtctcgtatgcgtcgtaaaatgtttttacgtcgtatgttacgtttaatttctcgtcgtactcgtcgtcctcgtttacgtgct

SEQ ID NO: 110 - DNA, 117 bp, Artificial Sequence
  Description of Artificial Sequence Synthetic polynucleotide
cgtactcgtaattttcgtattcgtcgtgctaaagctcgtcgtaaaatgatgttatctcattttaaatatggaatggctcgtaaaggatctaaatctcgttcttctcgtcgttctcgt

SEQ ID NO: 111 - DNA, 120 bp, Artificial Sequence
  Description of Artificial Sequence Synthetic polynucleotide
atggctcgttctcgtcgtcgtatgtctaaacgttcttctcgtcgttcttttcgtaaatatgctaaaactcataaacgtaattttaaagctcgttctatgcgtggaggaattcgtttataa

SEQ ID NO: 112 - DNA, 123 bp, Artificial Sequence; identical to SEQ ID NO: 32
  Description of Artificial Sequence Synthetic polynucleotide

SEQ ID NO: 113 - DNA, 117 bp, Artificial Sequence
  Description of Artificial Sequence Synthetic polynucleotide
gtgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 114 - DNA, 114 bp, Artificial Sequence
  Description of Artificial Sequence Synthetic polynucleotide
atggctaaaaaaatccgtaacaaagctcgtgaccgtcgtatcttcacccgtaccgcttctcgtatgcacaaagctaaccgtaccccgcgtttcatgcgtggtggtatccgtctg

SEQ ID NO: 115 - DNA, 120 bp, Artificial Sequence; identical to SEQ ID NO: 30
  Description of Artificial Sequence Synthetic polynucleotide

SEQ ID NO: 116 - DNA, 132 bp, Artificial Sequence
  Description of Artificial Sequence Synthetic polynucleotide
atgcgtctgcgttacggtcaccgtcgtatgaccgctggtcgtatccgtatgcgttctcgtcgtaaattcatgctgccgcgtttccgtctgctgcgtatcccgcgtcgttctaaccgtcgtcgtctgcgtgct

SEQ ID NO: 117 - DNA, 132 bp, Artificial Sequence; identical to SEQ ID NO: 116
  Description of Artificial Sequence Synthetic polynucleotide

SEQ ID NO: 118 - DNA, 117 bp, Artificial Sequence; identical to SEQ ID NO: 33
  Description of Artificial Sequence Synthetic polynucleotide

SEQ ID NO: 119 - DNA, 114 bp, Artificial Sequence; identical to SEQ ID NO: 45
  Description of Artificial Sequence Synthetic polynucleotide

SEQ ID NO: 120 - DNA, 114 bp, Artificial Sequence; identical to SEQ ID NO: 46
  Description of Artificial Sequence Synthetic polynucleotide

SEQ ID NO: 121 - DNA, 120 bp, Artificial Sequence; identical to SEQ ID NO: 47
  Description of Artificial Sequence Synthetic polynucleotide

SEQ ID NO: 122 - DNA, 114 bp, Artificial Sequence; identical to SEQ ID NO: 52
  Description of Artificial Sequence Synthetic polynucleotide

SEQ ID NO: 123 - DNA, 120 bp, Artificial Sequence; identical to SEQ ID NO: 68
  Description of Artificial Sequence Synthetic polynucleotide

SEQ ID NO: 124 - DNA, 117 bp, Artificial Sequence; identical to SEQ ID NO: 50
  Description of Artificial Sequence Synthetic polynucleotide

SEQ ID NO: 125 - protein, 4 aa, Artificial Sequence
  Description of Artificial Sequence Synthetic peptide
MIDR

SEQ ID NO: 126 - protein, 4 aa, Artificial Sequence
  Description of Artificial Sequence Synthetic peptide
FIRL

SEQ ID NO: 127 - protein, 4 aa, Artificial Sequence
  Description of Artificial Sequence Synthetic peptide
NPTH

SEQ ID NO: 128 - protein, 158 aa, Artificial Sequence
  Description of Artificial Sequence Synthetic polypeptide
MGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 129 - protein, 195 aa, Artificial Sequence
  Description of Artificial Sequence Synthetic polypeptide
  GN168 lysin
MRLKMARRRYRLPRRRSRRLFSRTALRMHPRNRLRRIMRGGIRFTAGGTAGGRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 130 - protein, 172 aa, Artificial Sequence
  Description of Artificial Sequence Synthetic polypeptide
  GN176 lysin lysin
MSFNVTPKFKRWQLYFRGRMWTAGGTAGGRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 131 - protein, 177 aa, Artificial Sequence
  Description of Artificial Sequence Synthetic polypeptide
  GN178 lysin lysin
MPPIFSKLAGKKIKNLLISGLKGGSGSGSGSGSPRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 132 - protein, 126 aa, Artificial Sequence
  Description of Artificial Sequence Synthetic polypeptide
  GN217 lysin
MTYTLSKRSLDNLKGVHPDLVAVVHRAIQLTPVDFAVIEGLRSVSRQKELVAAGASKTMNSRHLTGHAVDLAAYVNGIHWDWPLYDAIAVAVKAAAKELGVAIVWGGDWTTFKDGPHFELDRSKYR

SEQ ID NO: 133 - protein, 150 aa, Artificial Sequence
  Description of Artificial Sequence Synthetic polypeptide
  GN218 lysin
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYGGGSGGGGSGGGSRLKKIGKVLKWI

SEQ ID NO: 134 - protein, 174 aa, Artificial Sequence
  Description of Artificial Sequence Synthetic polypeptide
  GN223 lysin
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRPPGGGSGGGGSGGGSSKKASRKSFTKGAVKVHKKNVPTRVPMRGGIRL

SEQ ID NO: 135 - protein, 156 aa, Artificial Sequence
  Description of Artificial Sequence Synthetic polypeptide
  GN239 lysin
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYGGGSGGGGSGGGSRKKTRKRLKKIGKVLKWI

SEQ ID NO: 136 - protein, 173 aa, Artificial Sequence
  Description of Artificial Sequence Synthetic polypeptide
  GN243 lysin
MTYTLSKRSLDNLKGVHPDLVAVVHRAIQLTPVDFAVIEGLRSVSRQKELVAAGASKTMNSRHLTGHAVDLAAYVNGIRWDWPLYDAIAVAVKAAAKELGVAIVWGGDWTTFKDGPHFELDRSKYRKKTRKRLKKIGKVLKWIPPGGGSGGGGSGGGSTRKRLKKIGKVLKWI

SEQ ID NO: 137 - protein, 159 aa, Artificial Sequence
  Description of Artificial Sequence Synthetic polypeptide
  GN280 lysin
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQRKKTRKRLKKIGKVLKWIPPTAGGTAGGTRKRLKKIGKVLKWI

SEQ ID NO: 138 - protein, 164 aa, Artificial Sequence
  Description of Artificial Sequence Synthetic polypeptide
  GN281 lysin
MKLSEKRALFTQLLAQLILWAGTQDRVSVALDQVKRTQAEADANAKSGAGIRNSLHLLGLAGDLILYKDGKYMDKSEDYKFLGDYWKSLHPLCRWGGDFKSRPDGNHFSLEHEGVQRKKTRKRLKKIGKVLKWIGGGSGGGGSGGGSPPTRKRLKKIGKVLKWI

SEQ ID NO: 139 - protein, 264 aa, Artificial Sequence
  Description of Artificial Sequence Synthetic polypeptide
  GN316 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 140 - protein, 187 aa, Unknown
  Description of Unknown Pseudomonas phage KPP10 sequence
  GN329 lysin
MITDREYQQAAEMLGVDVPAIKAVTKVEAPVGGFQPTGEPTILYERHQMYRQLQAKGLPTEGHPPDLVNKVAGGYGKYSEQHAKLARAVKIDRDSALESCSWGMFQIMGYHWKLMGYPTLQAFVNAMYASEGAQMDAFCRFIKAQPTTHAALKAHDWAKFARLYNGPGYAKNKYDVKLEKAYAEASG

SEQ ID NO: 141 - protein, 186 aa, Delftia sp.
  GN333 lysin
MALTEQDFQSAADDLGVDVASVKAVTKVESRGSGFLLSGVPKILFERHWMFKLLKRKLGRDPEINDVCNPKAGGYLGGQAEHERLDKAVKMDRDCALQSASWGLFQIMGFHWEALGYASVQAFVNAQYASEGSQLNTFVRFIKTNPAIHKALKSKDWAEFARRYNGPDYKKNNYDVKLAEAYQSFK

SEQ ID NO: 142 - protein, 310 aa, Artificial Sequence
  Description of Artificial Sequence Synthetic polypeptide
  GN349 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGARRYRLSRRRSRRLFSRTALRMHRRNRLRRIMRGGIRF

SEQ ID NO: 143 - protein, 310 aa, Artificial Sequence
  Description of Artificial Sequence Synthetic polypeptide
  GN351 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGARSRRRMSKRSSRRSFRKYAKSHKKNFKARSMRGGIRL

SEQ ID NO: 144 - protein, 309 aa, Artificial Sequence
  Description of Artificial Sequence Synthetic polypeptide
  GN352 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGKRRKMTRKGSKRLFTATADKTKSINTAPPPMRGGIRL

SEQ ID NO: 145 - protein, 308 aa, Artificial Sequence
  Description of Artificial Sequence Synthetic polypeptide
  GN353 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGRKRMSKRVDKKVFRRTAASAKKINIDPKIYRGGIRL

SEQ ID NO: 146 - protein, 284 aa, Artificial Sequence
  Description of Artificial Sequence Synthetic polypeptide
  GN357 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGRRLIRLWLRLLR

SEQ ID NO: 147 - protein, 287 aa, Artificial Sequence
  Description of Artificial Sequence Synthetic polypeptide
  GN359 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSTAGGTAGGTRKRLKKIGKVLKWI

SEQ ID NO: 148 - protein, 282 aa, Artificial Sequence
  Description of Artificial Sequence Synthetic polypeptide
  GN369 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFSRKKTRKRLKKIGKVLKWI

SEQ ID NO: 149 - protein, 169 aa, Artificial Sequence
  Description of Artificial Sequence Synthetic polypeptide
  GN370 lysin
MIDRFIRLNPTHGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 150 - protein, 155 aa, Artificial Sequence
  Description of Artificial Sequence Synthetic polypeptide
  GN371 lysin
MIDRFIRLNPTHRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLDLLNKGDYQGAADQFPHWVNAGGKRLDGLVKRRAAERALFLEPLS

SEQ ID NO: 151 - protein, 264 aa, Artificial Sequence
  Description of Artificial Sequence Synthetic polypeptide
  GN394 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVDFIKADANLWKALKDKNWAEFARRYNGPAYAQNQYDTKLAAAYKSFS

SEQ ID NO: 152 - protein, 264 aa, Artificial Sequence
  Description of Artificial Sequence Synthetic polypeptide
  GN396 lysin
MAILKIGSKGLEVKNLQTSLNKIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWDALKDKNWAEFARRYNGPAYAQNQYDTKLAAAYKSFS

SEQ ID NO: 153 - protein, 264 aa, Artificial Sequence
  Description of Artificial Sequence Synthetic polypeptide
  GN408 lysin
MAILKIGSKGLEVKNLQTSLNKIGFNLVADGIFGKATDNAVRAVQAGAGLVVDGIAGPKTMYAIRNAGESHQDHLTEADLIDAAHELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 154 - protein, 264 aa, Artificial Sequence
  Description of Artificial Sequence Synthetic polypeptide
  GN418 lysin
MAILKIGSKGLEVKNLQTSLNDIGFNLVADGIFGKATDNAVRAVQAGAGLVVDGIAGPKTMYAIRNAGESHQDHLTEADLIDAARELSVDLASIKAVNQVESRGTGFTKSGKIKTLFERHIMYKKLNAKFGQAKANALAQLYPTLVNAKAGGYTGGDAELERLHGAIAIDKDCAYESASYGLFQIMGFNCVICGYDNAEEMFNDFLTGERAQLMAFVKFIKADANLWKALKDKNWAEFARRYNGPAYAQNQYDTKLAAAYKSFS

SEQ ID NO: 155 - protein, 269 aa, Burkholderia pseudomultivorans
  GN424 lysin
MNTLRFNSRGAEVGVLQQRLVRAGYPIDVTHLYDEATEQAVKALQAAAGIVVDGIAGPNTYAVLSAGQRDRKHLTEADIARAADKLGVSPACVRAVNEVESRGSGFLADGRPVILFERHVMYNRLVAAKRAVDAASAAQRFPNVVSAKPGGYQGGAAEYVRLDTAARIDAAIAYESASWGAFQVMGYHWERLGYSSIDEFVARMETSEGEQLDAFVRFVAADSSLRTALKNRKWAAFAKGYNGPDYARNLYDAKLAQAYERYAGTKAAA

SEQ ID NO: 156 - protein, 271 aa, Pseudomonas flexibilis
  GN425 lysin
MTLRLDDVGLDVLHLQKRLNELGANPRLLPDGQFGEVTERAVRAFQQRAGLVVDGVAGPKTMAALSGHSTSRLLGQRDLQRAADRLGVPLASVMALNAVESRGEGFAANGRPVILFERHVMHERLQVNGLSEAEADALAARHPGLVSRRPGGYVGDTAEHQRLANARLLHDTAALESASWGLFQVMGYHWQALGYDTTQDFTERMARHEAEHLEAFVRFIEADPALHKALKGRKWAEFARRYNGPAYARNLYDVKLARAFEQFSDALQAAA

SEQ ID NO: 157 - protein, 264 aa, Escherichia virus sp.
  GN428 lysin
MAILKLGNRGSEVKALQQSLNKIGFSLTADGIFGKATENAVKSVQAGAGLVIDGIAGPKTFYAIRNAGDAHQEHLTEADLVDAARELGVELASMKAVNQVESRGTGFTKTGKIKTLFERHIMYKKVTAKFGQARANALYQLYPTLVNPNSGGYIGGDAELERLQGAIALDEDCAYESASYGLFQIMGFNCQICGYSNAKEMFTDFLTGERAHLLAFVKFIKADANMWKALKNKNWAEFARRYNGPAYAKNQYDTKLAAAYKSFC

SEQ ID NO: 158 - protein, 209 aa, Artificial Sequence
  Description of Artificial Sequence Synthetic polypeptide
  GN93 lysin
MKFFKFFKFFKAGAGAGAGAGAGAGAGASNNELPWVAEARKYIGLREDTSKTSHNPKLLAMLDRMGEFSNESRAWWHDDETPWCGLFVGYCLGVAGRYVVREWYRARAWEAPQLTKLDRPAYGALVTFTRSGGGHVGFIVGKDARGNLMVLGGNQSNAVSIAPFAVSRVTGYFWPSFWRNKTAVKSVPFEERYSLPLLKSNGELSTNEA

SEQ ID NO: 159 - protein, 264 aa, Unknown
  Description of Unknown Dickeya phage phiD3 sequence
  GN431 lysin
MAILKLGNRGTEVKALQDSLNKIGFTLVADGIFGKATENAVKTVQAGAGLVIDGIVGPKTSYAIRNAGEAHQDHLTEADLIEAANQLGVDLASVKAVNQVESRGTGFTKSGKIKTLFERHIMYKKLMAKFGQARANAMGQMYPTLVSPVAGGYTGGDAELDRLHAAINIDEDCAYESASYGLFQIMGFNCQVCGYANAKEMFNDFLTGERAHLMAFVKFIKADAKLWQALKDKNWAEFARRYNGPAYTKNQYDTKLAAAYNSFN

SEQ ID NO: 160 - protein, 167 aa, Artificial Sequence
  Description of Artificial Sequence Synthetic polypeptide
  GN486 lysin
MGSHHHHHHGGPRRPRRPGRRAPVRTSQRGIDLIKSFEGLRLSAYQDSVGVWTIGYGTTRGVTRYMTITVEQAERMLSNDIQRFEPELDRLAKVPLNQNQWDALMSFVYNLGAANLASSTLLKLLNKGDYQGAADQFPRWVNAGGKRLDGLVKRRAAERALFLEPLS

SEQ ID NO: 161 - protein, 72 aa, Artificial Sequence
  Description of Artificial Sequence Synthetic polypeptide
  GN485 lysin
MPGLSGFIRNADTPVTSLGSAGHVHVPEGPLIRINPDCLLGTPFKFFKFFKFFKFFKFFKFFKFFKNECVLL